SEQUENCE LISTING (52 sequences)

SEQ ID NO: 1 - protein, 420 aa, Homo sapiens
  MOD_RES (116)..(116): Glu or Lys
MESPSAPPHRWCIPWQRLLLTASLLTFWNPPTTAKLTIESTPFNVAEGKEVLLLVHNLPQHLFGYSWYKGERVDGNRQIIGYVIGTQQATPGPAYSGREIIYPNASLLIQNIIQNXLSVDHSDPVILNVLYGPDDPTISPSYTYYRPGVNLSLSCHAASNPPAQYSWLIDGNIQQHTQELFISNITEKNSGLYTCQANNSASGHSRTTVKTITVSAELPKPSISSNNSKPVEDKDAVAFTCEPEAQNTTYLWWVNGQSLPVSPRLQLSNGNRTLTLFNVTRNDARAYVCGIQNSVSANRSDPVTLDVLYGPDTPIISPPDSSYLSGANLNLSCHSASNPSPQYSWRINGIPQQHTQVLFIAKITPNNNGTYACFVSNLATGRNNSIVKSITVSASGTSPGLSAGATVGIMIGVLVGVALI

SEQ ID NO: 2 - protein, 651 aa, Homo sapiens
  MOD_RES (364)..(364): Glu or Lys
KLTIESTPFNVAEGKEVLLLVHNLPQHLFGYSWYKGERVDGNRQIIGYVIGTQQATPGPAYSGREIIYPNASLLIQNIIQNDTGFYTLHVIKSDLVNEEATGQFRVYPELPKPSISSNNSKPVEDKDAVAFTCEPETQDATYLWWVNNQSLPVSPRLQLSNGNRTLTLFNVTRNDTASYKCETQNPVSARRSDSVILNVLYGPDAPTISPLNTSYRSGENLNLSCHAASNPPAQYSWFVNGTFQQSTQELFIPNITVNNSGSYTCQAHNSDTGLNRTTVTTITVYAEPPKPFITSNNSNPVEDEDAVALTCEPEIQNTTYLWWVNNQSLPVSPRLQLSNDNRTLTLLSVTRNDVGPYECGIQNXLSVDHSDPVILNVLYGPDDPTISPSYTYYRPGVNLSLSCHAASNPPAQYSWLIDGNIQQHTQELFISNITEKNSGLYTCQANNSASGHSRTTVKTITVSAELPKPSISSNNSKPVEDKDAVAFTCEPEAQNTTYLWWVNGQSLPVSPRLQLSNGNRTLTLFNVTRNDARAYVCGIQNSVSANRSDPVTLDVLYGPDTPIISPPDSSYLSGANLNLSCHSASNPSPQYSWRINGIPQQHTQVLFIAKITPNNNGTYACFVSNLATGRNNSIVKSITVSASGTSPGLSA

SEQ ID NO: 3 - protein, 356 aa, Artificial Sequence
  Description of Artificial Sequence Synthetic polypeptide
KLTIESTPFNVAEGKEVLLLVHNLPQHLFGYSWYKGERVDGNRQIIGYVIGTQQATPGPAYSGREIIYPNASLLIQNIIQNDLYGPDDPTISPSYTYYRPGVNLSLSCHAASNPPAQYSWLIDGNIQQHTQELFISNITEKNSGLYTCQANNSASGHSRTTVKTITVSAELPKPSISSNNSKPVEDKDAVAFTCEPEAQNTTYLWWVNGQSLPVSPRLQLSNGNRTLTLFNVTRNDARAYVCGIQNSVSANRSDPVTLDVLYGPDTPIISPPDSSYLSGANLNLSCHSASNPSPQYSWRINGIPQQHTQVLFIAKITPNNNGTYACFVSNLATGRNNSIVKSITVSASGTSPGLSA

SEQ ID NO: 4 - protein, 563 aa, Artificial Sequence
  Description of Artificial Sequence Synthetic polypeptide
KLTIESTPFNVAEGKEVLLLVHNLPQHLFGYSWYKGERVDGNRQIIGYVIGTQQATPGPAYSGREIIYPNASLLIQNIIQNDTGFYTLHVIKSDLVNEEATGQFRVYPELVLYGPDAPTISPLNTSYRSGENLNLSCHAASNPPAQYSWFVNGTFQQSTQELFIPNITVNNSGSYTCQAHNSDTGLNRTTVTTITVYAEPPKPFITSNNSNPVEDEDAVALTCEPEIQNTTYLWWVNNQSLPVSPRLQLSNDNRTLTLLSVTRNDVGPYECGIQNELSVDHSDPVILNVLYGPDDPTISPSYTYYRPGVNLSLSCHAASNPPAQYSWLIDGNIQQHTQELFISNITEKNSGLYTCQANNSASGHSRTTVKTITVSAELPKPSISSNNSKPVEDKDAVAFTCEPEAQNTTYLWWVNGQSLPVSPRLQLSNGNRTLTLFNVTRNDARAYVCGIQNSVSANRSDPVTLDVLYGPDTPIISPPDSSYLSGANLNLSCHSASNPSPQYSWRINGIPQQHTQVLFIAKITPNNNGTYACFVSNLATGRNNSIVKSITVSASGTSPGLSA

SEQ ID NO: 5 - protein, 568 aa, Artificial Sequence
  Description of Artificial Sequence Synthetic polypeptide
KLTIESTPFNVAEGKEVLLLVHNLPQHLFGYSWYKGERVDGNRQIIGYVIGTQQATPGPAYSGREIIYPNASLLIQNIIQNDTGFYTLHVIKSDLVNEEATGQFRVYPELPKPSISSNNSKPVEDKDAVAFTCEPETQDATYLWWVNNQSLPVSPRLQLSNGNRTLTLFNVTRNDTASYKCETQNPVSARRSDSVILNITVYAEPPKPFITSNNSNPVEDEDAVALTCEPEIQNTTYLWWVNNQSLPVSPRLQLSNDNRTLTLLSVTRNDVGPYECGIQNELSVDHSDPVILNVLYGPDDPTISPSYTYYRPGVNLSLSCHAASNPPAQYSWLIDGNIQQHTQELFISNITEKNSGLYTCQANNSASGHSRTTVKTITVSAELPKPSISSNNSKPVEDKDAVAFTCEPEAQNTTYLWWVNGQSLPVSPRLQLSNGNRTLTLFNVTRNDARAYVCGIQNSVSANRSDPVTLDVLYGPDTPIISPPDSSYLSGANLNLSCHSASNPSPQYSWRINGIPQQHTQVLFIAKITPNNNGTYACFVSNLATGRNNSIVKSITVSASGTSPGLSA

SEQ ID NO: 6 - protein, 556 aa, Artificial Sequence
  Description of Artificial Sequence Synthetic polypeptide
KLTIESTPFNVAEGKEVLLLVHNLPQHLFGYSWYKGERVDGNRQIIGYVIGTQQATPGPAYSGREIIYPNASLLIQNIIQNDTGFYTLHVIKSDLVNEEATGQFRVYPELPKPSISSNNSKPVEDKDAVAFTCEPETQDATYLWWVNNQSLPVSPRLQLSNGNRTLTLFNVTRNDTASYKCETQNPVSARRSDSVILNVLYGPDAPTISPLNTSYRSGENLNLSCHAASNPPAQYSWFVNGTFQQSTQELFIPNITVNNSGSYTCQAHNSDTGLNRTTVTTITVYAEPPTISPSYTYYRPGVNLSLSCHAASNPPAQYSWLIDGNIQQHTQELFISNITEKNSGLYTCQANNSASGHSRTTVKTITVSAELPKPSISSNNSKPVEDKDAVAFTCEPEAQNTTYLWWVNGQSLPVSPRLQLSNGNRTLTLFNVTRNDARAYVCGIQNSVSANRSDPVTLDVLYGPDTPIISPPDSSYLSGANLNLSCHSASNPSPQYSWRINGIPQQHTQVLFIAKITPNNNGTYACFVSNLATGRNNSIVKSITVSASGTSPGLSA

SEQ ID NO: 7 - protein, 473 aa, Artificial Sequence
  Description of Artificial Sequence Synthetic polypeptide
KLTIESTPFNVAEGKEVLLLVHNLPQHLFGYSWYKGERVDGNRQIIGYVIGTQQATPGPAYSGREIIYPNASLLIQNIIQNDTGFYTLHVIKSDLVNEEATGQFRVYPELPKPSISSNNSKPVEDKDAVAFTCEPETQDATYLWWVNNQSLPVSPRLQLSNGNRTLTLFNVTRNDTASYKCETQNPVSARRSDSVILNVLYGPDAPTISPLNTSYRSGENLNLSCHAASNPPAQYSWFVNGTFQQSTQELFIPNITVNNSGSYTCQAHNSDTGLNRTTVTTITVYAEPPKPFITSNNSNPVEDEDAVALTCEPEIQNTTYLWWVNNQSLPVSPRLQLSNDNRTLTLLSVTRNDVGPYECGIQNELSVDHSDPVILNVLYGPDTPIISPPDSSYLSGANLNLSCHSASNPSPQYSWRINGIPQQHTQVLFIAKITPNNNGTYACFVSNLATGRNNSIVKSITVSASGTSPGLSA

SEQ ID NO: 8 - protein, 214 aa, Artificial Sequence
  Description of Artificial Sequence Synthetic polypeptide
KLTIESTPFNVAEGKEVLLLVHNLPQHLFGYSWYKGERVDGNRQIIGYVIGTQQATPGPAYSGREIIYPNASLLIQNIIQNDTGFYTLHVIKSDLVNEEATGQFRVYPELKPFITSNNSNPVEDEDAVALTCEPEIQNTTYLWWVNNQSLPVSPRLQLSNDNRTLTLLSVTRNDVGPYECGIQNELSVDHSDPVILNVLYGPDDASGTSPGLSA

SEQ ID NO: 9 - protein, 212 aa, Artificial Sequence
  Description of Artificial Sequence Synthetic polypeptide
KLTIESTPFNVAEGKEVLLLVHNLPQHLFGYSWYKGERVDGNRQIIGYVIGTQQATPGPAYSGREIIYPNASLLIQNIIQNDTGFYTLHVIKSDLVNEEATGQFRVYPELKPSISSNNSKPVEDKDAVAFTCEPEAQNTTYLWWVNGQSLPVSPRLQLSNGNRTLTLFNVTRNDARAYVCGIQNSVSANRSDPVTLDVLYGPASGTSPGLSA

SEQ ID NO: 10 - protein, 702 aa, Homo sapiens
  MOD_RES (398)..(398): Glu or Lys
MESPSAPPHRWCIPWQRLLLTASLLTFWNPPTTAKLTIESTPFNVAEGKEVLLLVHNLPQHLFGYSWYKGERVDGNRQIIGYVIGTQQATPGPAYSGREIIYPNASLLIQNIIQNDTGFYTLHVIKSDLVNEEATGQFRVYPELPKPSISSNNSKPVEDKDAVAFTCEPETQDATYLWWVNNQSLPVSPRLQLSNGNRTLTLFNVTRNDTASYKCETQNPVSARRSDSVILNVLYGPDAPTISPLNTSYRSGENLNLSCHAASNPPAQYSWFVNGTFQQSTQELFIPNITVNNSGSYTCQAHNSDTGLNRTTVTTITVYAEPPKPFITSNNSNPVEDEDAVALTCEPEIQNTTYLWWVNNQSLPVSPRLQLSNDNRTLTLLSVTRNDVGPYECGIQNXLSVDHSDPVILNVLYGPDDPTISPSYTYYRPGVNLSLSCHAASNPPAQYSWLIDGNIQQHTQELFISNITEKNSGLYTCQANNSASGHSRTTVKTITVSAELPKPSISSNNSKPVEDKDAVAFTCEPEAQNTTYLWWVNGQSLPVSPRLQLSNGNRTLTLFNVTRNDARAYVCGIQNSVSANRSDPVTLDVLYGPDTPIISPPDSSYLSGANLNLSCHSASNPSPQYSWRINGIPQQHTQVLFIAKITPNNNGTYACFVSNLATGRNNSIVKSITVSASGTSPGLSAGATVGIMIGVLVGVALI

SEQ ID NO: 11 - protein, 10 aa, Homo sapiens
NIIQNELSVD

SEQ ID NO: 12 - protein, 10 aa, Homo sapiens
NIIQNKLSVD

SEQ ID NO: 13 - protein, 12 aa, Homo sapiens
QNIIQNELSVDH

SEQ ID NO: 14 - protein, 14 aa, Homo sapiens
IQNIIQNELSVDHS

SEQ ID NO: 15 - protein, 12 aa, Homo sapiens
QNIIQNKLSVDH

SEQ ID NO: 16 - protein, 14 aa, Homo sapiens
IQNIIQNKLSVDHS

SEQ ID NO: 17 - DNA, 25 bp, Artificial Sequence
  Description of Artificial Sequence Synthetic primer
cgcatacagtggtcgagagataata

SEQ ID NO: 18 - DNA, 24 bp, Artificial Sequence
  Description of Artificial Sequence Synthetic primer
cgctgtggtcaacacttaatttgt

SEQ ID NO: 19 - DNA, 18 bp, Artificial Sequence
  Description of Artificial Sequence Synthetic probe
atgcatccctgctgatcc

SEQ ID NO: 20 - DNA, 21 bp, Artificial Sequence
  Description of Artificial Sequence Synthetic primer
gaaacccagaacccagtgagt

SEQ ID NO: 21 - DNA, 24 bp, Artificial Sequence
  Description of Artificial Sequence Synthetic primer
gccatagaggacattcaggatgac

SEQ ID NO: 22 - DNA, 16 bp, Artificial Sequence
  Description of Artificial Sequence Synthetic probe
caggcgcagtgattca

SEQ ID NO: 23 - DNA, 40 bp, Artificial Sequence
  Description of Artificial Sequence Synthetic primer
taccgctagcgccaccatggagtctccctcggcccctccc

SEQ ID NO: 24 - DNA, 33 bp, Artificial Sequence
  Description of Artificial Sequence Synthetic primer
gctcgaattctcatatcagagcaaccaaccagc

SEQ ID NO: 25 - protein, 702 aa, Homo sapiens
MESPSAPPHRWCIPWQRLLLTASLLTFWNPPTTAKLTIESTPFNVAEGKEVLLLVHNLPQHLFGYSWYKGERVDGNRQIIGYVIGTQQATPGPAYSGREIIYPNASLLIQNIIQNDTGFYTLHVIKSDLVNEEATGQFRVYPELPKPSISSNNSKPVEDKDAVAFTCEPETQDATYLWWVNNQSLPVSPRLQLSNGNRTLTLFNVTRNDTASYKCETQNPVSARRSDSVILNVLYGPDAPTISPLNTSYRSGENLNLSCHAASNPPAQYSWFVNGTFQQSTQELFIPNITVNNSGSYTCQAHNSDTGLNRTTVTTITVYAEPPKPFITSNNSNPVEDEDAVALTCEPEIQNTTYLWWVNNQSLPVSPRLQLSNDNRTLTLLSVTRNDVGPYECGIQNELSVDHSDPVILNVLYGPDDPTISPSYTYYRPGVNLSLSCHAASNPPAQYSWLIDGNIQQHTQELFISNITEKNSGLYTCQANNSASGHSRTTVKTITVSAELPKPSISSNNSKPVEDKDAVAFTCEPEAQNTTYLWWVNGQSLPVSPRLQLSNGNRTLTLFNVTRNDARAYVCGIQNSVSANRSDPVTLDVLYGPDTPIISPPDSSYLSGANLNLSCHSASNPSPQYSWRINGIPQQHTQVLFIAKITPNNNGTYACFVSNLATGRNNSIVKSITVSASGTSPGLSAGATVGIMIGVLVGVALI

SEQ ID NO: 26 - protein, 420 aa, Homo sapiens
MESPSAPPHRWCIPWQRLLLTASLLTFWNPPTTAKLTIESTPFNVAEGKEVLLLVHNLPQHLFGYSWYKGERVDGNRQIIGYVIGTQQATPGPAYSGREIIYPNASLLIQNIIQNELSVDHSDPVILNVLYGPDDPTISPSYTYYRPGVNLSLSCHAASNPPAQYSWLIDGNIQQHTQELFISNITEKNSGLYTCQANNSASGHSRTTVKTITVSAELPKPSISSNNSKPVEDKDAVAFTCEPEAQNTTYLWWVNGQSLPVSPRLQLSNGNRTLTLFNVTRNDARAYVCGIQNSVSANRSDPVTLDVLYGPDTPIISPPDSSYLSGANLNLSCHSASNPSPQYSWRINGIPQQHTQVLFIAKITPNNNGTYACFVSNLATGRNNSIVKSITVSASGTSPGLSAGATVGIMIGVLVGVALI

SEQ ID NO: 27 - protein, 5 aa, Artificial Sequence
  Description of Artificial Sequence Synthetic penta-His tag
HHHHH

SEQ ID NO: 28 - protein, 5 aa, Artificial Sequence
  Description of Artificial Sequence Synthetic peptide
FYFDY

SEQ ID NO: 29 - protein, 10 aa, Artificial Sequence
  Description of Artificial Sequence Synthetic peptide
DXXXXFYFDY

SEQ ID NO: 30 - protein, 6 aa, Artificial Sequence
  Description of Artificial Sequence Synthetic peptide
RFYFDY

SEQ ID NO: 31 - protein, 7 aa, Artificial Sequence
  Description of Artificial Sequence Synthetic peptide
LRFYFDY

SEQ ID NO: 32 - protein, 8 aa, Artificial Sequence
  Description of Artificial Sequence Synthetic peptide
GLRFYFDY

SEQ ID NO: 33 - protein, 9 aa, Artificial Sequence
  Description of Artificial Sequence Synthetic peptide
RGLRFYFDY

SEQ ID NO: 34 - protein, 10 aa, Artificial Sequence
  Description of Artificial Sequence Synthetic peptide
DRGLRFYFDY

SEQ ID NO: 35 - protein, 4 aa, Artificial Sequence
  Description of Artificial Sequence Synthetic peptide
RGLR

SEQ ID NO: 36 - protein, 5 aa, Artificial Sequence
  Description of Artificial Sequence Synthetic peptide
SYWMH

SEQ ID NO: 37 - protein, 18 aa, Artificial Sequence
  Description of Artificial Sequence Synthetic peptide
FIRNKANGGTTEYMSVKG

SEQ ID NO: 38 - protein, 18 aa, Artificial Sequence
  Description of Artificial Sequence Synthetic peptide
FILNKANGGTTEYMSVKG

SEQ ID NO: 39 - protein, 5 aa, Artificial Sequence
  Description of Artificial Sequence Synthetic peptide
TYAMH

SEQ ID NO: 40 - protein, 17 aa, Artificial Sequence
  Description of Artificial Sequence Synthetic peptide
LISNDGSNKYYADSVKG

SEQ ID NO: 41 - protein, 14 aa, Artificial Sequence
  Description of Artificial Sequence Synthetic peptide
TLRRGINVGAYSIY

SEQ ID NO: 42 - protein, 11 aa, Artificial Sequence
  Description of Artificial Sequence Synthetic peptide
YKSDSDKQQGS

SEQ ID NO: 43 - protein, 10 aa, Artificial Sequence
  Description of Artificial Sequence Synthetic peptide
MIWHSGASAV

SEQ ID NO: 44 - protein, 19 aa, Artificial Sequence
  Description of Artificial Sequence Synthetic peptide
FILNKANGGTTEYAASVKG

SEQ ID NO: 45 - DNA, 1506 bp, Artificial Sequence
  Description of Artificial Sequence Synthetic A240 VL bispecific single chain antibody polynucleotide
caggccgtgctgactcagccggcttccctctctgcatctcctggagcatcagccagtctcacctgcaccttgcgcaggggcatcaatgttggtgcctacagtatatactggtaccagcagaagccagggagtcctccccagtatctcctgaggtacaaatcagactcagataagcagcagggctctggagtctccagccgcttctctgcatccaaagatgcttcggccaatgcagggattttactcatctctgggctccagtctgaggatgaggctgactattactgtatgatttggcacagcggcgcttctgcggtgttcggcggagggaccaagttgaccgtcctaggtggtggtggttctggcggcggcggctccggtggtggtggttctgaggtgcagctggtcgagtctgggggaggcttggtccagcctgggaggtccctgagactctcctgtgcagcgtctggattcaccgtcagtagctactggatgcactgggtccgccaagctccagggaaggggctggaatgggtaggtttcattagaaacaaagctaatggtgggacaacagaatacgccgcgtctgtgaaaggcagattcaccatctcaagagatgattccaagaacacgctgtatcttcaaatgaacagcctgagagccgaggacacggccgtgtattactgtgcaagagatagggggctacggttctactttgactactggggccaagggaccacggtcaccgtctcctcatccggaggtggtggatccgacgtccaactggtgcagtcaggggctgaagtgaaaaaacctggggcctcagtgaaggtgtcctgcaaggcttctggctacacctttactaggtacacgatgcactgggtaaggcaggcacctggacagggtctggaatggattggatacattaatcctagccgtggttatactaattacgcagacagcgtcaagggccgcttcacaatcactacagacaaatccaccagcacagcctacatggaactgagcagcctgcgttctgaggacactgcaacctattactgtgcaagatattatgatgatcattactgccttgactactggggccaaggcaccacggtcaccgtctcctcaggcgaaggtactagtactggttctggtggaagtggaggttcaggtggagcagacgacattgtactgacccagtctccagcaactctgtctctgtctccaggggagcgtgccaccctgagctgcagagccagtcaaagtgtaagttacatgaactggtaccagcagaagccgggcaaggcacccaaaagatggatttatgacacatccaaagtggcttctggagtccctgctcgcttcagtggcagtgggtctgggaccgactactctctcacaatcaacagcttggaggctgaagatgctgccacttattactgccaacagtggagtagtaacccgctcacgttcggtggcgggaccaaggtggagatcaaatag

SEQ ID NO: 46 - protein, 501 aa, Artificial Sequence
  Description of Artificial Sequence Synthetic A240 VL bispecific single chain antibody polypeptide
QAVLTQPASLSASPGASASLTCTLRRGINVGAYSIYWYQQKPGSPPQYLLRYKSDSDKQQGSGVSSRFSASKDASANAGILLISGLQSEDEADYYCMIWHSGASAVFGGGTKLTVLGGGGSGGGGSGGGGSEVQLVESGGGLVQPGRSLRLSCAASGFTVSSYWMHWVRQAPGKGLEWVGFIRNKANGGTTEYAASVKGRFTISRDDSKNTLYLQMNSLRAEDTAVYYCARDRGLRFYFDYWGQGTTVTVSSSGGGGSDVQLVQSGAEVKKPGASVKVSCKASGYTFTRYTMHWVRQAPGQGLEWIGYINPSRGYTNYADSVKGRFTITTDKSTSTAYMELSSLRSEDTATYYCARYYDDHYCLDYWGQGTTVTVSSGEGTSTGSGGSGGSGGADDIVLTQSPATLSLSPGERATLSCRASQSVSYMNWYQQKPGKAPKRWIYDTSKVASGVPARFSGSGSGTDYSLTINSLEAEDAATYYCQQWSSNPLTFGGGTKVEIK

SEQ ID NO: 47 - protein, 19 aa, Artificial Sequence
  Description of Artificial Sequence Synthetic peptide
FIRNKANGGTTEYAASVKG

SEQ ID NO: 48 - protein, 116 aa, Artificial Sequence
  Description of Artificial Sequence Synthetic polypeptide
QAVLTQPASLSASPGASASLTCTLRRGINVGAYSIYWYQQKPGSPPQYLLRYKSDSDKQQGSGVSSRFSASKDASANAGILLISGLQSEDEADYYCMIWHSGASAVFGGGTKLTVL

SEQ ID NO: 49 - protein, 121 aa, Artificial Sequence
  Description of Artificial Sequence Synthetic polypeptide
EVQLVESGGGLVQPGRSLRLSCAASGFTVSSYWMHWVRQAPGKGLEWVGFIRNKANGGTTEYAASVKGRFTISRDDSKNTLYLQMNSLRAEDTAVYYCARDRGLRFYFDYWGQGTTVTVSS

SEQ ID NO: 50 - protein, 243 aa, Artificial Sequence
  Description of Artificial Sequence Synthetic polypeptide
DVQLVQSGAEVKKPGASVKVSCKASGYTFTRYTMHWVRQAPGQGLEWIGYINPSRGYTNYADSVKGRFTITTDKSTSTAYMELSSLRSEDTATYYCARYYDDHYCLDYWGQGTTVTVSSGEGTSTGSGGSGGSGGADDIVLTQSPATLSLSPGERATLSCRASQSVSYMNWYQQKPGKAPKRWIYDTSKVASGVPARFSGSGSGTDYSLTINSLEAEDAATYYCQQWSSNPLTFGGGTKVEIK

SEQ ID NO: 51 - protein, 121 aa, Artificial Sequence
  Description of Artificial Sequence Synthetic polypeptide
EVQLVESGGGLVQPGRSLRLSCAASGFTVSSYWMHWVRQAPGKGLEWVGFILNKANGGTTEYAASVKGRFTISRDDSKNTLYLQMNSLRAEDTAVYYCARDRGLRFYFDYWGQGTTVTVSS

SEQ ID NO: 52 - protein, 507 aa, Artificial Sequence
  Description of Artificial Sequence Synthetic polypeptide
QAVLTQPASLSASPGASASLTCTLRRGINVGAYSIYWYQQKPGSPPQYLLRYKSDSDKQQGSGVSSRFSASKDASANAGILLISGLQSEDEADYYCMIWHSGASAVFGGGTKLTVLGGGGSGGGGSGGGGSEVQLVESGGGLVQPGRSLRLSCAASGFTVSSYWMHWVRQAPGKGLEWVGFIRNKANGGTTEYAASVKGRFTISRDDSKNTLYLQMNSLRAEDTAVYYCARDRGLRFYFDYWGQGTTVTVSSSGGGGSDVQLVQSGAEVKKPGASVKVSCKASGYTFTRYTMHWVRQAPGQGLEWIGYINPSRGYTNYADSVKGRFTITTDKSTSTAYMELSSLRSEDTATYYCARYYDDHYCLDYWGQGTTVTVSSGEGTSTGSGGSGGSGGADDIVLTQSPATLSLSPGERATLSCRASQSVSYMNWYQQKPGKAPKRWIYDTSKVASGVPARFSGSGSGTDYSLTINSLEAEDAATYYCQQWSSNPLTFGGGTKVEIKHHHHHH

(19) United States
(12) Patent Application Publication
(10) Pub. No.: US 2014/0242081 A1
(43) Pub. Date: Aug. 28, 2014

(54) DOSING REGIMENS FOR TREATMENT OF CEA-EXPRESSING CANCERS

(71) Applicants: Scott Hammond, Gaithersburg, MD (US); Patricia Ryan, Gaithersburg, MD (US); Song Ren, Gaithersburg, MD (US); Petra Lutterbuese, Neuried (DE); Maria Amann, Zurick (CH)

(72) Inventors: Scott Hammond, Gaithersburg, MD (US); Patricia Ryan, Gaithersburg, MD (US); Song Ren, Gaithersburg, MD (US); Petra Lutterbuese, Neuried (DE); Maria Amann, Zurick (CH)

(73) Assignees: MICROMET AG, Munich (DE); MEDIMMUNE, LLC, Gaithersburg, MD (US)

(21) Appl. No.: 14/233,406

(22) PCT Filed: Jul. 18, 2011
(86) PCT No.: PCT/US11/44339

§ 371 Date: May 12, 2014

(51) Int. Cl. C07K 16/28

(52) U.S. Cl. CPC C07K 16/2809
USPC 424/136.1; 424/172.1

(57) ABSTRACT

The present disclosure provides compositions and methods for treating CEA-expressing cancers. Methods for dosing a patient with an antibody that binds to CEA and human CD3 are also provided.

### BACKGROUND

The present application relates to treatment of cancers that express carcinoembryonic antigen (CEA). CEA is a glycosylated human oncofetal antigen that belongs to the CEA-related cell adhesion molecule (CEACAM) family of the immunoglobulin gene superfamily. CEA has been suggested to mediate cell-cell adhesion, facilitate bacterial colonization of the intestine, and protect the colon from microbial infection by binding and trapping infectious microorganisms. Carcinoembryonic antigen (CEA) is a well-characterized tumor-associated antigen that is frequently over-expressed in human carcinomas and melanomas.

Carcinoembryonic antigen has been widely used as a target for both tumor imaging and various antibody-based therapeutic approaches for cancer treatment. One therapeutic approach makes use of a bispecific single-chain antibody that (1) targets human CEA on tumor cells, and (2) targets the CD3 epsilon (ε) subunit of the human T-cell receptor complex present on T cells. The pharmacological action of this class of antibodies, known as a bispecific T-cell engager (BITE® antibody), is based on their ability to mediate T-cell lysis of target-expressing tumor cells. Nonclinical in vitro studies of human tumor cell lines expressing CEA and in vivo studies using animal tumor models have demonstrated that a BiTE® antibody called MEDI-565 (also known as MT-111) has potent antitumor cell activity and growth inhibition, and antitumor activity is not inhibited by soluble CEA.

To date, pharmacological testing of MEDI-565 has been limited. Because MEDI-565 is specific for human CEA and human CD3, there is no pharmacologically relevant animal species for toxicology testing of MEDI-565. Hybrid surrogate molecules were generated in order to develop a pharmacologically relevant animal species model for predicting human toxicity, but the pharmacodynamic characteristics of these molecules differed from those of MEDI-565. Accordingly, there remains a need for methods to estimate a Minimal Anticipated Biological Effect Level (MABEL) to use as a starting dose for MEDI-565 administration, and, moreover, a need for effective but safe doses of MEDI-565.

### SUMMARY OF THE DISCLOSURE

The present disclosure provides various methods for treating a CEA-expressing cancer. In a first aspect, the disclosure provides a method for treating a CEA-expressing cancer. The method comprises administering to a human patient in need of treatment an antibody (or a protein composition comprising an antibody). In either case, the antibody comprises a first binding domain that binds to human CD3 and a second binding domain that binds to human CEA. The antibody is provided at a dose of about 0.75 μg to about 10 mg per day, or even at a dose of greater than about 10 mg per day, on a dosing schedule comprising administering the protein composition once per day for at least one day.

In a second aspect, the disclosure provides a method for treating a CEA-expressing cancer. The method comprises administering to a human patient in need of treatment an antibody (or a protein composition comprising an antibody). In either case, the antibody comprises a first binding domain that binds to human CD3 and a second binding domain that binds to human CEA. The protein composition is administered on a dosing schedule and at a dose of antibody that maintains a serum concentration of the antibody in the patient of at least 0.1 ng/mL.

In a third aspect, the disclosure provides a method for treating a CEA-expressing cancer. The method comprises administering to a patient in need thereof a composition comprising an antibody comprising a first binding domain that binds to human CD3 and a second binding domain that binds to human CEA at a dose of antibody and on a dosing schedule sufficient to maintain a serum concentration of antibody that is therapeutically effective and sufficient to lyse at least about 20%, 25%, 30%, 35%, 40%, 45%, 50%, 55%, 60% or more of the cancerous cells that express CEA.

The following features described below (or in the detailed description or examples) should be understood to apply to any of the foregoing aspects of the disclosure. Moreover, the disclosure contemplates that any one or more of these embodiments may be combined. In certain embodiments, the dosing schedule is part of a treatment cycle of 21 or 28 days.

In certain embodiments, the CEA-expressing cancer is chosen from: colon cancer, colorectal cancer, ovarian cancer, prostate cancer, rectal cancer, pancreatic cancer, esophageal cancer, gastroesophageal cancer, stomach cancer, lung cancer and breast cancer. In certain embodiments, the CEA-expressing cancer is a relapsed or refractory cancer. In certain embodiments, the CEA-expressing cancer is an adenocarcinoma of gastrointestinal origin.

In certain embodiments, the antibody is a bispecific single chain antibody comprising a first binding domain that binds to human CD3 and a second binding domain that binds to the human CEA. In certain embodiments, the antibody or bispecific single chain antibody comprises an amino acid sequence chosen from the amino acid sequences of SEQ ID NOs: 28-44 and 46-52. In certain embodiments, the antibody comprises the amino acid sequence of SEQ ID NO: 48. In certain embodiments, the antibody comprises the amino acid sequence of SEQ ID NO: 49. In certain embodiments, the antibody comprises the amino acid sequence of SEQ ID NO: 46. In certain embodiments, the antibody comprises the amino acid sequence of SEQ ID NO: 51. In certain embodiments, the antibody comprises the amino acid sequence of SEQ ID NO: 52.

In certain embodiments, the antibody is provided at a dose of about 0.75 μg to about 2.25 μg per day. In other embodiments, the antibody is provided at a dose of about 2.25 μg to about 6.75 μg per day. In other embodiments, the antibody is provided at a dose of about 6.75 μg to about 20 μg per day. In still other embodiments, the antibody is provided at a dose of about 20 μg to about 60 μg per day. In other embodiments, the antibody is provided at a dose of about 60 μg to about 180 μg per day. In other embodiments, the antibody is provided at a dose of about 180 μg to about 540 μg per day. In other embodiments, the antibody is provided at a dose of about 540 μg to about 1.5 mg per day. In still other embodiments, the antibody is provided at a dose of about 1.5 mg to about 3 mg per day. In other embodiments, the antibody is provided at a dose of about 1.5 mg per day. In other embodiments, the antibody is provided at a dose of about 3 mg to about 5 mg per day. In certain embodiments, the antibody is provided at a dose of about 5 mg to about 7.5 mg per day. In other embodiments, the antibody is provided at a dose of about 7.5 mg to about 10 mg per day. In still other embodiments, the antibody is provided at a dose of greater than about 10 mg per day.

In other embodiments, the protein composition (or antibody) is administered intravenously, such as by intravenous infusion. In certain embodiments, administration by intravenous infusion is over a period of, for example, about 1, 1.5, 2, 2.5, 3, or more, hours.

In certain embodiments, the dosing schedule comprises administration once per day for at least 2, 3, 4, or 5 consecutive days.

In certain embodiments, the method comprises one than one treatment cycle, such as more than one treatment cycle where each cycle is 21 days.

In certain embodiments, the method comprises one than one treatment cycle, such as more than one treatment cycle where each cycle is 28 days.

In certain embodiments, the patient is administered the same dose of the antibody in the protein composition each day of administration. In other embodiments, the dose differs, such as a higher dose is administered during a second treatment cycle relative to a first or a higher dose is administered on day three than on day 1 or 2 of administration within a treatment cycle. Similarly, in other embodiments, the dose may be lower during a second treatment cycle relative to a first or a lower dose may be administered on day three relative to day 1 or 2. The foregoing are merely exemplary of ways in which dose of antibody may differ during or between treatment cycles.

In certain embodiments, the patient receives a therapeutically effective dose sufficient to lyse at least about 20%, 25%, 30%, 35%, 40%, 45%, 50%, 55%, or 60% of the cancerous cells that express CEA. In other embodiments, the patient receives a therapeutically effective dose sufficient to lyse greater than about 60% of the cancerous cells that express CEA.

In certain embodiments, the patient receives a therapeutically effective dose sufficient to increase release of one or more pro-inflammatory cytokines, perforin, and/or granzyme by at least about 25%, 30%, 35%, 40%, 45%, or 50% relative to untreated cells. In other embodiments, the patient receives a therapeutically effective dose sufficient to increase release of one or more pro-inflammatory cytokines, perforin, and/or granzyme by greater than 50% relative to untreated cells. In certain embodiments, the one or more proinflammatory cytokines are chosen from IFNγ, TNFα, IL-2, IL-12ₚ₇₀, IL-1β, IL-4, IL-6, IL-8, IL-10, and IL-13.

In certain embodiments, the patient receives a therapeutically effective dose sufficient to reduce tumor volume by at least about 20% or at least about 25%, as compared to untreated control tumors. In other embodiments, the patient receives a therapeutically effective dose sufficient to reduce tumor volume by greater than about 25%, such as at least about 30%, 40% or 50%, as compared to untreated control tumors.

In certain embodiments, the patient receives a therapeutically effective dose sufficient to increase expression of T cell activation markers CD69 and/or CD25 by at least about 20% or at least about 25%, relative to untreated cells. In other embodiments, the patient receives a therapeutically effective dose sufficient to increase expression of T cell activation markers CD69 and/or CD25 by greater than about 25%, such as at least about 30%, 40% or 50%, relative to untreated cells.

In certain embodiments, the patient receives a therapeutically effective dose sufficient to induce proliferation of peripheral blood mononuclear cells, particularly CD3+ T cells (PBMCs with CD38).

In certain embodiments of any of the foregoing, the method further comprises measuring therapeutic efficacy, wherein a measured change in the patient between an earlier time point and a subsequent time point indicates that the protein composition is therapeutically effective. In certain embodiments, the measured change is chosen from at least one of increased lysis of cells that express CEA; increased release of one or more pro-inflammatory cytokines, perforin and/or granzyme; decreased tumor volume; increased T cell activation; and increased proliferation of peripheral blood mononuclear cells, particularly CD3+ T cells; fractional receptor occupancy. The first time point may be, for example, prior to administration of any antibody of the disclosure, after the first day of administration, after the fifth day of administration, at the beginning of a treatment cycle, at the end of a treatment cycle, etc. Regardless of when the first time point is, the second time point is subsequent to the first time point.

In certain embodiments, the dosing schedule maintains the antibody at a serum concentration in the patient above a selected concentration chosen from 2 ng/ml, 4, ng/ml, 6.67 ng/ml, 10 ng/ml and 13.3 ng/ml.

In certain embodiments, the dosing schedule maintains the antibody at a serum concentration between about 0.1 ng/mL to about 2 ng/mL in the patient for at least 4 hours. In certain embodiments, the dosing schedule maintains the antibody at a serum concentration between about 0.1 ng/mL to about 2 ng/mL in the patient for at least 24 hours. In other embodiments, the dosing schedule maintains the antibody at a serum concentration between about 0.1 ng/mL to about 2 ng/mL in the patient for at least one week.

In certain embodiments, the dosing schedule maintains the antibody at a serum concentration above about 2 ng/ml in the patient for at least 4 hours. In certain embodiments, the dosing schedule maintains the antibody at a serum concentration above about 2 ng/ml in the patient for at least 24 hours. In certain embodiments, the dosing schedule maintains the antibody at a serum concentration above about 2 ng/ml in the patient for at least one week.

In certain embodiments, the dosing schedule maintains the antibody at a serum concentration above about 4 ng/ml in the patient for at least 4 hours. In certain embodiments, the dosing schedule maintains the antibody at a serum concentration above about 4 ng/ml in the patient for at least 24 hours. In certain embodiments, the dosing schedule maintains the antibody at a serum concentration above about 4 ng/ml in the patient for at least one week.

In certain embodiments, the dosing schedule maintains the antibody at a serum concentration above about 7 ng/ml in the patient for at least 4 hours. In certain embodiments, the dosing schedule maintains the antibody at a serum concentration above about 7 ng/ml in the patient for at least 24 hours. In certain embodiments, the dosing schedule maintains the antibody at a serum concentration above about 7 ng/ml in the patient for at least one week.

In certain embodiments, the dosing schedule maintains the antibody at a serum concentration above about 10 ng/ml in the patient for at least 4 hours. In certain embodiments, the dosing schedule maintains the antibody at a serum concentration above about 10 ng/ml in the patient for at least 24 hours. In certain embodiments, the dosing schedule maintains the antibody at a serum concentration above about 10 ng/ml in the patient for at least one week.

In certain embodiments, the dosing schedule maintains the antibody at a serum concentration above about 13 ng/ml in the patient for at least 4 hours. In certain embodiments, the dosing schedule maintains the antibody at a serum concentration above about 13 ng/ml in the patient for at least 24 hours. In certain embodiments, the dosing schedule maintains the antibody at a serum concentration above about 13 ng/ml in the patient for at least one week.

In addition, the disclosure contemplates a protein composition comprising an antibody comprising a first binding domain that binds to human CD3 and a second binding domain that binds to human CEA for use in treating a CEA-expressing cancer, wherein the antibody is administered at a dose of about 0.75 μg to about 10 mg per day on a dosing schedule comprising administering the protein composition once per day for at least one day. Any of the features described above or described in the detailed description and examples may, in certain embodiments, be used to describe such a use.

In another aspect, the disclosure provides a protein composition comprising an antibody comprising a first binding domain that binds to human CD3 and a second binding domain that binds to human CEA for use in treating a CEA-expressing cancer by administration of about 0.75 μg to about 10 mg of antibody per day on a dosing schedule in which the protein composition is administered once per day for at least one day. Any of the features described above or described in the detailed description and examples may, in certain embodiments, be used to describe such a use.

In another aspect, the disclosure provides a protein composition comprising an antibody comprising a first binding domain that binds to human CD3 and a second binding domain that binds to human CEA for use in treating a CEA-expressing cancer, wherein the protein composition is administered on a dosing schedule and at a dose of antibody that maintains a serum concentration of the protein composition of at least about 0.1 ng/mL. Any of the features described above or described in the detailed description and examples may, in certain embodiments, be used to describe such a use.

In another aspect, the disclosure provides an antibody comprising a first binding domain that binds to human CD3 and a second binding domain that binds to human CEA for use in treating a CEA-expressing cancer, wherein the antibody is administered at a dose of about 0.75 μg to about 10 mg per day on a dosing schedule comprising administering the antibody once per day for at least one day. Any of the features described above or described in the detailed description and examples may, in certain embodiments, be used to describe such a use.

In still another aspect, the disclosure provides an antibody comprising a first binding domain that binds to human CD3 and a second binding domain that binds to human CEA for use in treating a CEA-expressing cancer by administration of about 0.75 μg to about 10 mg of antibody per day on a dosing schedule in which the antibody is administered once per day for at least one day. Any of the features described above or described in the detailed description and examples may, in certain embodiments, be used to describe such a use.

The disclosure contemplates that any of the aspects and embodiments of the disclosure may be combined. Moreover, the disclosure contemplates that any one or more embodiments of the disclosure may be combined.

### BRIEF DESCRIPTION OF THE DRAWINGS

FIG. 1 shows the effect of the effector cell to target cell ratio (E:T ratio) on MEDI-565-mediated redirected T-cell lysis of Chinese Hamster Ovary (CHO)/human CEA (huCEA) cells. 3,3′-dioctadecyloxacarbocyanine (DiO)-labeled CHO/huCEA cells were mixed with peripheral blood mononuclear cells (PBMC) enriched for CD3-positive T cells (PBMCs with CD38) at different E:T cell ratios in the presence of titrated doses of MEDI-565. After 72 hours, target cell lysis was determined by means of propidium iodide (PI) incorporation. The specific lysis and the percentage of expression of CD25 and CD69 on T cells of samples containing 10 μg/mL MEDI-565 for different E:T ratios are shown in (A). The EC₅₀ values calculated for different E:T ratios is shown in (B). Each data point represents the mean of duplicate wells. Error bars represent standard error of the mean. The experiment was performed twice with similar results.

FIG. 2 shows the kinetics of MEDI-565-induced T cell activation and lysis of CHO/huCEA cells. Human PBMC enriched for CD3-positive T cells (“CD3 enriched PBMC” or “PBMC with CD3γ”; B, D, and F) or depleted of CD14-positive cells (“PBMC w/o CD14”; A, C, and E) were cultured in the presence of CHO/huCEA cells at an E:T cell ratio of 10:1 with medium, 10 μg/mL of MEDI-565, or control BiTE® antibody for up to 72 hours. Additionally, pure target or effector cells were cultivated for up to 72 hours. Target cell lysis after different incubation periods was monitored by flow cytometric analysis of PI uptake (A, B). T cell activation was monitored by flow cytometric determination of de novo expression of CD69 (C, D) or CD25 (E, F) at different time points after start of culture. Error bars show the standard error of the mean. h=hour.

FIG. 3 shows the kinetics of MEDI-565-mediated cytokine release using CHO/huCEA cells and PBMC without CD14 and PBMC with CD3ε effector cells. Human PBMC without CD14 (A, B, and C) or PBMC with CD3ε (D, E, and F) were cultured in the presence of CHO/huCEA cells at an E:T cell ratio of 10:1 with 5 or 10 μg/mL of MEDI-565 (triangle), or vehicle (circle; A, B and C), or control BiTE® antibody (circle; D, E, and F) for up to 72 hours. The supernatant was analyzed for the mean concentrations of IFNγ (A, D), IL-2 (B, E), and TNFα (C, F) at different time points after start of culture using the cytokine/chemokine Milliplex MAP kit (A, B, and C) and Luminex xMAP technology platform (D, E, and F). Error bars represent the standard error of the mean. The experiment was performed with two different donors and produced similar results. h=hour.

FIG. 4 shows the effect of the E:T ratio on MEDI-565-mediated redirected T cell lysis of ASPC-1 cells. DiO-labeled ASPC-1 cells were mixed with human PBMC without CD14 at the indicated E:T ratios in the presence of serial dilutions of MEDI-565. After 48 hours, target cell lysis was determined by means of PI incorporation. The specific lysis and the percentage of expression of CD25 and CD69 on T cells of samples containing 10 μg/mL of MEDI-565 for different E:T ratios is shown. The calculated EC₅₀ values for different E:T ratios is also shown. Each data point represents the mean of duplicate wells. Error bars represent the standard error of the mean. The experiment was performed two times with similar results.

FIG. 5 shows kinetics of MEDI-565-induced T cell activation and lysis of ASPC-1 cells. Human PBMC without CD14 (left panels) and with CD3ε (right panels), respectively, were cultured in the presence of ASPC-1 cells at an E:T cell ratio of 5:1 with 10 μg/mL MEDI-565 (filled triangle), 10 μg/mL control BITE® antibody (filled diamond), or vehicle (filled circle), or in the absence of ASPC-1 cells (open circle) for up to 72 hours. Additionally, target cells were cultivated without effector cells for up to 72 hours (open triangle). Target cell lysis after different incubation periods was monitored by flow cytometric analysis of PI uptake (A, B). T cell activation was monitored by flow cytometric determination of de novo expression of CD69 (C, D) or CD25 (E, F) at different time points after start of culture. Error bars show the standard error of the mean. The experiment was performed with two (PBMC without CD14) and four (PBMC with CD3ε) different donors, respectively, obtaining similar results. h=hour.

FIG. 6 shows kinetics of MEDI-565-mediated cytokine release using ASPC-1 cells. Human PBMC without CD14 (left panels) and with CD3ε (right panels), respectively, were cultured in the presence of ASPC-1 cells at an E:T cell ratio of 5:1 with 10 μg/mL MEDI-565 (filled circle), 10 μg/mL control BiTE® antibody (filled square), or vehicle (open triangle) for up to 72 hours. The supernatant was analyzed for IFNγ (A, B), TNFα (C, D), and IL-10 (E, F) at the indicated time points after start of culture using the CBA Human Th1/Th2 Cytokine Kit II. Error bars show the standard error of the mean. The experiment was performed with two (PBMC without CD14) and four (PBMC with CD3ε) different donors, respectively, obtaining similar results. h=hour.

FIG. 7 shows the MABEL determination for MEDI-565-induced specific lysis of CHO/huCEA cells and de novo expression of the T cell activation markers CD25 and CD69. MEDI-565 bioactivity is shown after a 72-hour incubation period with PBMC without CD14 (left panels) and PBMC with CD3ε (right panels) with CHO/huCEA tumor cells at an E:T ratio of 10:1 with increasing concentrations of MEDI-565 or the control BiTE® antibody. Specific lysis was monitored by flow cytometric analysis of PI uptake (A, B). T cell activation was monitored by flow cytometric determination of de novo expression of CD69 (C, D) or CD25 (E, F). Error bars indicate standard error of the mean of duplicate determinations.

FIG. 8 shows the MABEL determination for MEDI-565-induced specific lysis of ASPC-1 cells and de novo expression of the T cell activation markers CD25 and CD69. MEDI-565 bioactivity is shown after a 48-hour incubation period of PBMC without CD14 (right panels) and PBMC with CD3ε (left panels), respectively, with ASPC-1 tumor cells at an E:T ratio of 5:1 with increasing concentrations of MEDI-565 or the control BiTE® antibody. Specific lysis was monitored by flow cytometric analysis of PI uptake (A, B). T cell activation was monitored by flow cytometric determination of de novo expression of CD69 (C, D) or CD25 (E, F). Error bars indicate the standard error of the mean of duplicate determinations. Note: For PBMC with CD3ε only, the curves meeting the inclusion criteria (see Material and Methods) are shown.

FIG. 9 shows the MABEL of MEDI-565 for specific lysis of target cells and CD69 and CD25 de novo expression on T cells. EC₅₀ (A) and EC₂₀ (B) values of specific lysis of target cells and CD69 and CD25 de novo expression on T cells are shown for PBMC without CD14 and with CD3ε, respectively, cultured with both CHO/huCEA and ASPC-1 tumor cells in the presence of MEDI-565. Specific lysis of the target cells was monitored by flow cytometric analysis of PI uptake. T cell activation was monitored by flow cytometric determination of de novo expression of CD69 or CD25. Error bars indicate the standard error of the mean. Each symbol represents an effector cell population isolated from a unique healthy donor. Legend: A=ASPC-1; P=PBMC without CD14; C=CHO/huCEA; 3=PBMC with CD3ε.

FIG. 10 shows MEDI-565-mediated cytokine release using ASPC-1 cells. PBMC with CD3ε from 7 different donors were cultured in the presence of ASPC-1 cells at an E:T cell ratio of 5:1 with increasing concentrations of MEDI-565 and control BiTE® antibody for 48 hours. Culture supernatants were analyzed for IFNγ (A), IL-10 (B), IL-2 (C), and TNFα (D) concentrations using the CBA Human Th1/Th2 Cytokine Kit II. Error bars show the standard error of the mean.

FIG. 11 shows influence of overnight effector cell culture on cytokine levels. Human PBMC without CD14 (left panels) and with CD3ε (right panels) were cultivated overnight (o.n.) in medium containing either 10% FBS (fetal bovine serum) or 10% of the human donor-matched plasma. Thereafter, the effector cells were cultured in the presence of ASPC-1 target cells at an E:T cell ratio of 5:1 with 10 μg/mL MEDI-565 (C, D) or vehicle control (E, F) for 48 hours. The supernatants before (A, B) and after (C, D, E, F) the assays were analyzed for IFNγ, TNFα, IL-6, and IL-10 using the CBA Human Th1/Th2 Cytokine Kit II. Error bars show the standard error of the mean. The experiment was performed with effector cells from six (PBMC without CD14) or two (PBMC with CD3ε) different healthy donors, respectively, and resulted in similar findings.

FIG. 12 shows the influence of overnight effector cell culture on T cell activation and target cell lysis. Human PBMC without CD14 (left panel) and with CD3ε (right panel) were cultivated overnight (o.n.) in medium containing either 10% FBS or 10% of the donor-matched plasma. Thereafter, the effector cells were cultured in the presence of ASPC-1 target cells at an E:T cell ratio of 5:1 with the indicated concentrations of MEDI-565 for 48 hours. Specific lysis of target cells was monitored by flow cytometric analysis of PI uptake (A, B). T cell activation was monitored by flow cytometric determination of de novo expression of CD25 (C, D) or CD69 (E, F). Error bars show the standard error of the mean. The experiment was performed with effector cells from six (PBMC without CD14) or two (PBMC with CD3ε) different healthy donors, respectively, and resulted in similar findings.

FIG. 13 shows the specificity of MEDI-565 induced T cell activation and tumor cell killing. (A) Effects of MEDI-565 on specific lysis of ASPC-1 tumor cells in the absence of effector cells were analyzed by flow cytometry following a 48-hour incubation period. (B to D) Redirected T cell lysis of ASPC-1 cells (B) and T cell activation (C, D) by the control BiTE® antibody at an E:T ratio of 5:1 were analyzed by flow cytometry following a 48-hour incubation period. Ten different preparations of donor PBMC with CD3ε were tested and are depicted by different symbols. After 48 hours, reactions were stopped, and tumor cell lysis was analyzed by flow cytometry via uptake of PI (A, B). T cell activation was monitored with fluorescent-labeled antibodies against CD25 (C) and CD69 (D). Error bars indicate standard error of the mean.

FIG. 14 shows the MABEL determination for MEDI-565-induced upregulation of the T cell activation markers CD69 and CD25 and specific lysis of ASPC-1 tumor cells. T cell activation (expression of activation markers CD69 and CD25) and specific lysis of tumor cells are shown after a 48-hour incubation period of PBMC with CD3ε combined with ASPC-1 tumor cells at an E:T ratio of 5:1 in the presence of serial dilutions of MEDI-565. T cell activation was monitored with fluorescent-labeled antibodies against CD69 and CD25. Tumor cell lysis was analyzed by flow cytometry via uptake of PI. (A to C) Using GraphPad Prism 4 software (Graph Pad Software, San Diego), the percentage of CD69—(A) or CD25-positive T cells (B) and specific lysis (C) were plotted against BiTE® antibody concentration. Error bars indicate standard error of the mean of duplicate determinations. (D) Dose-response curves of each donor were analyzed with a four parametric logistic regression model for evaluation of sigmoid dose response curves with variable Hill slope, and EC₅₀ and EC₂₀ values were calculated. Each symbol represents an effector cell population isolated from a unique healthy donor.

FIG. 15 shows the MABEL determination for MEDI-565-induced cytokine release. The cytokine release was analyzed after 48 hours of incubation of PBMC with CD3ε combined with ASPC-1 tumor cells at an E:T ratio of 5:1 in the presence of serial dilutions of MEDI-565. The supernatant was analyzed for IL-2 (A), IL-6 (B), IL-10 (C), TNFα (D), and IFNγ (E) using the Human Cytokine/Chemokine Milliplex MAP Kit and Luminex xMAP technology platform. Error bars show the standard error of the mean.

FIG. 16 shows the fractional receptor occupancy of MEDI-565 bound to CD3 and huCEA target antigens. Based on the equation (F=[mAb]/([mAb]+K_{D}], the fraction (F) of all receptor molecules that are bound to the respective ligand has been calculated for both the CD3 target antigen (A) as well as the huCEA target antigen (B). Lines indicate tolerable receptor occupancy of 20% of the maximum amount.

FIG. 17 shows the simulated human serum concentration-time profiles of MEDI-565 following 0.75 μg or 1.5 mg of MEDI-565 administered as a 3-hour intravenous (IV) infusion once daily for 5 consecutive days.

### DETAILED DESCRIPTION

I. Bispecific Antibodies that Bind to Human CEA and Human CD3

Carcinoembryonic antigen (CEA; CEACAM5) is a glycosylated human oncofetal antigen that belongs to the CEA-related cell adhesion molecule (CEACAM) family of the immunoglobulin gene superfamily. CEACAM5 is closely related to CEACAM1, CEACAM3, CEACAM4, CEACAM6, CEACAM7 and CEACAM8. CEA has been suggested to mediate cell-cell adhesion, facilitate bacterial colonization of the intestine, and protect the colon from microbial infection by binding and trapping infectious microorganisms. As used herein, CEA refers to CEACAM5, particularly human CEACAM5.

CEA is expressed at low levels in normal tissues of epithelial origin (Hammarstrom, 1999) in a polarized manner, and such expression is only observed at the luminal portion of the cell. In contrast, expression of CEA is high in carcinomas (including colon, pancreatic, gastric, esophageal, lung, breast, uterine, ovarian, and endometrial) and in a subset of melanomas (Hammarstrom, 1999; Sanders et al., 1994). Cancer cells not only lose polarized (luminal) expression of CEA, but actively cleave CEA from their surface by phospholipases, an action that results in high serum levels of CEA (Hammarstrom, 1999).

Serum levels of CEA serve as a useful prognostic indicator in patients with gastrointestinal cancers (Duffy, 2001, Locker et al., 2006; Rother, 2007); elevated levels indicate a poor prognosis and correlate with reduced overall survival. Detection of CEA-expression is described in WO2011/068758, the entire contents of which are incorporated herein by reference.

By convention, amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

The numbering of amino acids in the variable domain, complementarity determining region (CDRs) and framework regions (FR), of an antibody follow, unless otherwise indicated, the Kabat definition as set forth in Kabat et al. Using this numbering system, the actual linear amino acid sequence may contain fewer or additional amino acids corresponding to a shortening of, or insertion into, a FR or CDR of the variable domain. For example, a heavy chain variable domain may include a single amino acid insertion (residue 52a according to Kabat) after residue 52 of H2 and inserted residues (e.g. residues 82a, 82b, and 82c, etc according to Kabat) after heavy chain FR residue 82. The Kabat numbering of residues may be determined for a given antibody by alignment at regions of homology of the sequence of the antibody with a “standard” Kabat numbered sequence. Maximal alignment of framework residues frequently requires the insertion of “spacer” residues in the numbering system, to be used for the Fv region. In addition, the identity of certain individual residues at any given Kabat site number may vary from antibody chain to antibody chain due to interspecies or allelic divergence.

As used herein, the terms “antibody” and “antibodies”, also known as immunoglobulins, encompass monoclonal antibodies (including full-length monoclonal antibodies), polyclonal antibodies, multispecific antibodies formed from at least two different epitope binding fragments (e.g., bispecific antibodies), human antibodies, humanized antibodies, camelised antibodies, chimeric antibodies, single-chain Fvs (scFv), single-chain antibodies, single domain antibodies, domain antibodies, Fab fragments, F(ab′)2 fragments, antibody fragments that exhibit the desired biological activity (e.g. the antigen binding portion), disulfide-linked Fvs (dsFv), and anti-idiotypic (anti-Id) antibodies (including, e.g., anti-Id antibodies to antibodies of the disclosure), intrabodies, and epitope-binding fragments of any of the above. In particular, antibodies include immunoglobulin molecules and immunologically active fragments of immunoglobulin molecules, i.e., molecules that contain at least one antigen-binding site. Immunoglobulin molecules can be of any isotype (e.g., IgG, IgE, IgM, IgD, IgA and IgY), subisotype (e.g., IgG1, IgG2, IgG3, IgG4, IgA1 and IgA2) or allotype (e.g., Gm, e.g., G1m(f, z, a or x), G2m(n), G3m(g, b, or c), Am, Em, and Km(1, 2 or 3)). Antibodies may be derived from any mammal, including, but not limited to, humans, monkeys, pigs, horses, rabbits, dogs, cats, mice, etc., or other animals such as birds (e.g. chickens).

As used herein, the term “immunoglobulin-like molecule” refers to an antibody mimic or antibody-like scaffold. In certain embodiments, immunoglobulin-like molecules may be any polypeptide comprising a non-immunoglobulin antigen binding scaffold, including, single chain antibodies, diabodies, minibodies, etc. Immunoglobulin-like molecules may contain an immunoglobulin-like fold. In certain aspects, the immunoglobulin-like molecules may be derived from a reference protein by having a mutated amino acid sequence. In certain embodiments, the immunoglobulin-like molecule may be derived from an antibody substructure, minibody, adnectin, anticalin, affibody, knottin, glubody, C-type lectin-like domain protein, tetranectin, kunitz domain protein, thioredoxin, cytochrome b562, zinc finger scaffold, Staphylococcal nuclease scaffold, fibronectin or fibronectin dimer, tenascin, N-cadherin, E-cadherin, ICAM, titin, GCSF-receptor, cytokine receptor, glycosidase inhibitor, antibiotic chromoprotein, myelin membrane adhesion molecule PO, CD8, CD4, CD2, class I MHC, T-cell antigen receptor, CD1, C2 and I-set domains of VCAM-1,1-set immunoglobulin domain of myosin-binding protein C, 1-set immunoglobulin domain of myosin-binding protein H, 1-set immunoglobulin domain of telokin, NCAM, twitchin, neuroglian, growth hormone receptor, erythropoietin receptor, prolactin receptor, interferon-gamma receptor, β-galactosidase/glucuronidase, β-glucuronidase, transglutaminase, T-cell antigen receptor, superoxide dismutase, tissue factor domain, cytochrome F, green fluorescent protein, GroEL, or thaumatin.

As used herein, the term “A5B7” refers to a mouse monoclonal antibody immunospecific for CEA and described in, for example, WO07/071,426, Barnett et al., Boxer et al., Harwood et al., Fidarova et al., and Nap et al.

As used herein, the term “MEDI-565” refers to a bispecific single chain antibody, known as a BiTE® antibody, that includes an anti-CEA binding portion and an anti-CD3 binding portion. The anti-CEA binding portion is a humanized scFv derived from mouse monoclonal antibody A5B7. MEDI-565 is described and disclosed in WO07/071,426, Lutterbuese et al., 2009, Journal of Immunother 32: 341-352, and Osada et al. 2010 Br J Cancer 102:124-133. The term “BiTE”, is a registered trademark of Micromet AG, and refers to a class of antibody or antibody-like molecules also known as bi-specific T-cell engagers. Such molecules have a portion that is immunospecific for an antigen associated with a diseased state (e.g., an antigen expressed on cancerous cells) and a portion that links such a diseased cell to T cells. WO07/071,426 provides additional exemplary description of BiTE® type molecules. The contents of WO07/071,426 are incorporated by reference herein in their entirety.

The disclosure provides methods of treating a CEA-expressing cancer using a particular dosing scheme. Any of the antibodies of the disclosure having a first portion that binds to human CD3 and a second portion that binds to human CEA can be used in any of the disclosed methods of treatment. In certain embodiments, the therapeutic regimen comprises treatment with a bispecific antibody (including a bispecific single chain antibody) that includes both an anti-CEA portion and an anti-CD3 portion. In certain embodiments, the therapeutic to be used with the methods of the disclosure is MEDI-565. Specific methods for treating with such bispecific antibodies, including MEDI-565, are found in PCT publication WO2007/071426, incorporated herein by reference in its entirety. See also, Lutterbuese et al., 2009, Journal of Immunotherapy 32: 341-352, Osada et al. 2010, British Journal of Cancer, 102: 124-33, and Medical News Today (www.medicalnewstoday.com/articles/145690.php), each of which describe MEDI-565 and are incorporated by reference in their entirety. In certain embodiments, the therapeutic to be used includes, at least, a CEA binding portion that binds to the same or substantially the same epitope as MEDI-565.

In certain embodiments, the therapeutic to be used includes, at least, a CEA binding portion comprising the amino acid sequence represented in any of SEQ ID NOs: 28-44 and 46-52. In certain embodiments, the therapeutic to be used is a bispecific antibody comprising the amino acid sequence represented in any of SEQ ID NOs: 28-44 and 47. In certain embodiments, the therapeutic to be used is a bispecific antibody comprising the amino acid sequence represented in any of SEQ ID NOs: 34, 36, 41, 42, 43, and 47. In certain embodiments, the therapeutic to be used is a bispecific antibody comprising the amino acid sequence represented in any of SEQ ID NOs: 37-40. In certain embodiments, the therapeutic to be used is a bispecific antibody comprising the amino acid sequence represented in SEQ ID NO: 48. In certain embodiments, the therapeutic to be used is a bispecific antibody comprising the amino acid sequence represented in SEQ ID NO: 49. In certain embodiments, the therapeutic to be used is a bispecific antibody comprising the amino acid sequence represented in SEQ ID NOs: 48 and 49. In certain embodiments, the therapeutic to be used is a bispecific antibody comprising the amino acid sequence represented in SEQ ID NO: 46. In certain embodiments, the therapeutic to be used is a bispecific antibody comprising the amino acid sequence represented in SEQ ID NO: 52.

In certain embodiments, the therapeutic to be used is a bispecific antibody, such as a bispecific single chain antibody. The order of arrangement of the first and second binding domains, such as within the bispecific antibody or bispecific single chain antibody, is relevant. It is envisaged that the arrangement of the binding domains may be VH_{CEA}-VL_{CEA}-VH_{CD3}-VL_{CD3}, VL_{CEA}-VH_{CEA}-VH_{CD3}-VL_{CD3}, VH_{CD3}-VL_{CD3}-VH_{CEA}-VL_{CEA} or VH_{CD3}-VL_{CD3}-VL_{CEA}-VH_{CEA}. In some examples, the first binding domain specifically binding to human CD3 is arranged in the VH-VL orientation. For example, the binding domains of the bispecific single chain antibodies defined herein may be arranged in the order VH_{CEA}-VL_{CEA}-VH_{CD3}-VL_{CD3} or VL_{CEA}-VH_{CEA}-VH_{CD3}-VL_{CD3}. As used in this context, “N-terminally to” or “C-terminally to” and grammatical variants thereof denote relative location within the primary amino acid sequence rather than placement at the absolute N- or C-terminus of a molecule. Hence, as a non-limiting example, a first binding domain which is “located C-terminally to the second binding domain” simply denotes that the first binding domain is located to the carboxyl side of the second binding domain within the bispecific antibody, and does not exclude the possibility that an additional sequence, for example a tag as set forth above, or another proteinaceous or non-proteinaceous compound such as a radioisotope, is located at the ultimate C-terminus of the bispecific antibody.

In certain embodiments, the therapeutic is a bispecific antibody or a single chain bispecific antibody with binding domains arranged in the order VH_{CEA}-VL_{CEA}-VH_{CD3}-VL_{CD3} or VL_{CEA}-VH_{CEA}-VH_{CD3}-VL_{CD3}. In certain embodiments, the arrangement is VL_{CEA}-VH_{CEA}-VH_{CD3}-VL_{CD3}. In certain embodiments, the therapeutic is a bispecific single chain antibody construct A240 VL-B9 VH×SEQ ID NO. 50 VHVL as defined in SEQ ID NO. 46. In certain embodiments, the therapeutic to be used is a bispecific antibody comprising the amino acid sequence represented in SEQ ID NO: 52.

In some examples, the binding domain specifically binding to human CEA of the bispecific antibody or bispecific single chain antibody comprises at least one CDR, such as a CDR-H3, such as a part of the CDR-H3 of murine monoclonal antibody A5B7 with the amino acid sequence “FYFDY” (SEQ ID NO. 28) corresponding to Kabat positions 100, 100a, 100b, 101, and 102, respectively, of CDR-H3 of murine monoclonal antibody A5B7. In some examples, the CDH-H3 has the amino acid sequence “DX₁X₂X₃X₄FYFDY” (SEQ ID NO. 29), wherein “X, “X₂”, “X₃” or “X₄” represents any amino acid residue, and the amino acid residue “D” corresponds to Kabat position 95 of CDR-H3 of murine monoclonal antibody A5B7 and the amino acid residues “FYFDY” correspond to Kabat positions 100, 100a, 100b, 101, and 102, respectively, of CDR-H3 of murine monoclonal antibody A5B7. Herein, “X₁”, “X₂”, “X₃” and “X₄” correspond to Kabat positions 96 (“X₁”), 97 (“X₂”), 98 (“X₃”) and 99 (“X₄”), respectively, of CDR-H3 of murine monoclonal antibody A5B7. It is envisaged that “X₁”, “X₂”, “X₃” or “X₄” represent amino acid residue “R” (Arginine), “G” (Glycine), “L” (Leucine), “Y” (Tyrosine), “A” (Alanine), “D” (Aspartic acid), “S” (Serine), “W” (Tryptophan), “F” (Phenylalanine) or “T” (Threonine). In certain embodiments, it is excluded from the scope of the disclosure that “X₁”, “X₂”, “X₃” and “X₄” represent the same amino acid, e.g. that “X₁”, “X₂”, “X₃” and “X₄”, are all “F” (Phenylalanine) In certain embodiments, “X₁” represents “R” (Arginine), “F” (Phenylalanine), “M” (Methionine), “E” (Glutamic acid), or “T” (Threonine); “X₂” represents “G” (Glycine), “Y” (Tyrosine), “A” (Alanine), “D” (Aspartic acid), or “S” (Serine); “X₃” represents “L” (Leucine), “F” (Phenylalanine), “M” (Methionine), “E” (Glutamic acid), or “T” (Threonine); and “X₄” represents “R” (Arginine), “Y” (Tyrosine), “A” (Alanine), “D” (Aspartic acid), or “S” (Serine).

In some examples, the second binding domain specific for human CEA comprises at least the amino acid sequence “RFYFDY” (SEQ ID NO. 30), “LRFYFDY” (SEQ ID NO. 31), “GLRFYFDY” (SEQ ID NO. 32), or “RGLRFYFDY” (SEQ ID NO. 33) of CDR-H3 of monoclonal antibody A5B7. In some examples, the second binding domain comprises the complete CDR-H3 of A5B7 with the amino acid sequence “DRGLRFYFDY” (SEQ ID NO. 34) corresponding to Kabat positions 95 (“D”, Aspartic acid), 96 (“R”; Arginine), 97 (“G”; Glycine), 98 (“L”; Leucine), 99 (“R”; Arginine), 100 (“F”; Phenylalanine), 100a (“Y”; Tyrosine), 100b (“F”; Phenylalanine), 101 (“D”; Aspartic acid), and 102 (“Y”; Tyrosine), respectively. Numbering according to the Kabat system is set forth e.g. in Kabat et al.

In certain embodiments, it may be desirable to further modify this A5B7-derived “DRGLRFYFDY” (SEQ ID NO: 34) CDR-H3 amino acid sequence e.g. in order to improve affinity for the CEA target antigen (on the epithelial tumor cells) and/or to optimize “fine specificity” of the bispecific single chain antibody as defined herein. To this end, in the amino acid sequence “DX₁X₂X₃X₄FYFDY” (SEQ ID NO. 29), various amino acid residues may be tested at positions “X₁”, “X₂”, “X₃” and/or “X₄” (corresponding to Kabat positions 96 (“X₁”), 97 (“X₂”), 98 (“X₃”) and 99 (“X₄”), respectively, of CDR-H3 of murine monoclonal antibody A5B7) in order to identify a modified CDR-H3 with improved affinity and/or fine specificity. For instance, “X₁”, “X₂”, “X₃” or “X₄” may represent amino acid residue “R” (Arginine), “G” (Glycine), “L” (Leucine), “Y” (Tyrosine), “A” (Alanine), “D” (Aspartic acid), “S” (Serine), “W” (Tryptophan), “F” (Phenylalanine) or “T” (Threonine). Herein, one, two, three or all four of the indicated “X” positions may be exchanged in comparison to the original “RGLR” amino acid sequence at Kabat positions 96 to 99 in the CDR-H3 “DRGLRFYFDY” (SEQ ID NO. 34) amino acid sequence. In certain embodiments, it is excluded that “X₁”, “X₂”, “X₃” and “X₄” represent the same amino acid, e.g. that “X₁”, “X₂”, “X₃” and “X₄” are all “F” (Phenylalanine) The above-mentioned modification of the A5B7-derived “DRGLRFYFDY” CDR-H3 amino acid sequence can be achieved by methods known in the art, such as PCR using randomized primers, which allows the generation of bispecific single chain antibodies with such modified CDR-H3 regions in the CEA-binding domain. Affinity or fine specificity of these modified bispecific single chain antibodies can be tested by methods described in the art, e.g. by ELISA, Biacore or FACS analysis.

In some embodiments, the binding domain specific for human CEA of the therapeutic agent, such as a bispecific single chain antibody, comprises a CDR-H1 having the amino acid sequence “SYWMH” (SEQ ID NO. 36) and/or a CDR-H2 having the amino acid sequence “FIRNKANGGTTEYMSVKG” (SEQ ID NO. 37) or “FILNKANGGTTEYMSVKG” (SEQ ID NO. 38).

In some embodiments, the binding domain specific for human CEA of the therapeutic agent, such as a bispecific single chain antibody, comprises a CDR-H1 having the amino acid sequence “SYWMH” (SEQ ID NO. 36) and/or a CDR-H2 having the amino acid sequence “FIRNKANGGTTEYMSVKG” (SEQ ID NO. 37) or “FIRNKANGGTTEYAASVKG” (SEQ ID NO. 47).

Alternatively, said second binding domain specific for human CEA of the bispecific single chain antibodies defined herein comprises a CDR-H1 having the amino acid sequence “TYAMH” (SEQ ID NO. 39) and/or a CDR-H2 having the amino acid sequence “LISNDGSNKYYADSVKG” (SEQ ID NO. 40).

In certain embodiments, the amino acid sequence of the VH region of the binding domain specific for human CEA, such as of a bispecific antibody or a bispecific single chain antibody comprises “DRGLRFYFDY” (SEQ ID NO. 34) corresponding to Kabat positions 95-102 of the CDR-H3 of murine monoclonal antibody A5B7 and a CDR-H1 having the amino acid sequence “SYWMH” (SEQ ID NO. 36) and a CDR-H2 having the amino acid sequence “FIRNKANGGTTEYMSVKG” (SEQ ID NO. 37).

In certain embodiments, the amino acid sequence of the VH region of the binding domain specific for human CEA, such as in a bispecific format or a bispecific single chain format, is SEQ ID NO. 146 comprising “DRGLRFYFDY” (SEQ ID NO. 34) corresponding to Kabat positions 95-102 of the CDR-H3 of murine monoclonal antibody A5B7 and a CDR-H1 having the amino acid sequence “SYWMH” (SEQ ID NO. 36) and a CDR-H2 having the amino acid sequence “FILNKANGGTTEYAASVKG” (SEQ ID NO. 44).

In certain embodiments, the amino acid sequence of the VH region of the binding domain specific for human CEA, such as in a bispecific format or a bispecific single chain format, comprises “DRGLRFYFDY” (SEQ ID NO. 34) corresponding to Kabat positions 95-102 of the CDR-H3 of murine monoclonal antibody A5B7 and a CDR-H1 having the amino acid sequence “SYWMH” (SEQ ID NO. 36) and a CDR-H2 having the amino acid sequence “FIRNKANGGTTEYAASVKG” (SEQ ID NO. 47).

In certain embodiments, the amino acid sequence of the VH region of the binding domain specific for human CEA, such as in a bispecific single chain format, comprises “DRGLRFYFDY” (SEQ ID NO. 34) corresponding to Kabat positions 95-102 of the CDR-H3 of murine monoclonal antibody A5B7 and a CDR-H1 having the amino acid sequence “TYAMH” (SEQ ID NO. 39) and a CDR-H2 having the amino acid sequence “LISNDGSNKYYADSVKG” (SEQ ID NO. 40).

In certain embodiments, the amino acid sequence of the VH region of the binding domain specific for human CEA comprises an amino acid sequence having the sequence of SEQ ID NO: 59.

Thus, said binding domain specific for human CEA of, for example a bispecific single chain antibody, may comprise one, two or three CDR-H regions as defined above.

In certain embodiments, the amino acid sequence of the VL region of the binding domain specific for human CEA, such as in a bispecific format or a bispecific single chain format, comprises CDR-L1 having the amino acid sequence “TLRRGINVGAYSIY” (SEQ ID NO. 41) and a CDR-L2 having the amino acid sequence “YKSDSDKQQGS” (SEQ ID NO. 42 and a CDR-L3 having the amino acid sequence “MIWHSGASAV” (SEQ ID NO. 43).

In certain embodiments, the amino acid sequence of the VH region of the binding domain specific for human CEA comprises an amino acid sequence having the sequence of SEQ ID NO: 48.

As noted above, the order or arrangement of the variable regions of the second binding domain specifically binding to CEA may be VH-VL or VL-VH.

In certain embodiments, the V regions of the CEA binding portion of a therapeutic agent, such as a therapeutic bispecific antibody, or a bispecific single chain antibody is chosen from:

(a) the VH region consists of the amino acid sequence shown in SEQ ID NO. 49 and the VL region consists of the amino acid sequence shown in SEQ ID NO. 48;

(b) the VH region consists of the amino acid sequence shown in SEQ ID NO. 51 and the VL region consists of the amino acid sequence shown in SEQ ID NO. 48;

In certain embodiments, the therapeutic is a bispecific single chain antibody comprising an amino acid sequence chosen from:

(a) an amino acid sequence as depicted in any of SEQ ID NOs. 28-52

(b) an amino acid sequence encoded by a nucleic acid sequence encoding any of SEQ ID NOs. 28-52;

(c) an amino acid sequence encoded by a nucleic acid sequence hybridizing under stringent conditions to the complementary nucleic acid sequence of (b);

(d) an amino acid sequence encoded by a nucleic acid sequence which is degenerate as a result of the genetic code to a nucleotide sequence of (b); and

(e) an amino acid sequence at least 85% identical, at least 90% identical, or at least 95% identical to the amino acid sequence of (a) or (b).

In certain embodiments, the therapeutic is a bispecific single chain antibody comprising the amino acid sequence of SEQ ID NO: 46.

### II. Determining a Minimal Anticipated Biological Effect Level (MABEL) of Bispecific Antibodies

One aspect of the disclosure relates to administration of antibodies that are specific for CEA, for use in treating CEA-expressing cancers. For example, a bispecific single-chain antibody described herein (such as MEDI-565) binds human CEA on cancer cells and the human CD38/T cell receptor complex present on all human T cells. The result of such binding is T cell-mediated killing of human cancer cells expressing human CEA.

Because bispecific antibodies such as MEDI-565 are specific for human CD3 and human CEA, these molecules do not bind to orthologous proteins in other species commonly used for safety testing. Therefore, toxicity studies to extrapolate a safe starting dose in man, based on the classical “No Observed Effect Level” (NOEL) or “No Observed Adverse Effect Level” (NOAEL) derived from toxicity studies in a relevant animal species, cannot be performed with these molecules. Because bispecific antibodies modulate immune function, it is crucial to ensure that these molecules do not lead to adverse effects, such as non-specific activation of T cells, T cell infiltration of organs, and/or a cytokine storm. Thus, a minimal anticipated biological effect level (MABEL) for a given therapeutic must be determined.

In some embodiments, the calculation of MABEL is based on (i) receptor binding and receptor occupancy based on in vitro studies in target cells from human and relevant animal(s) species, in vivo studies in relevant animal species, and known ligand binding affinities (ii) concentration-response curves from in vitro studies in target cells from human and relevant animal(s) species, and dose-response curves from in vivo studies in relevant animal species, and (iii) exposures at pharmacological doses in relevant species. In further embodiments, an in vitro dose response analysis based on T cell activation, tumor cell lysis, and cytokine release data may be used to generate dose-response curves using a human tumor cell line and human effector cell preparations from different donors. MABEL may be defined by measuring the effective concentration of a bispecific antibody such as MEDI-565, for example, the effective concentration that induced 20% of a maximal effect (EC₂₀ values), or 50% maximal effect (EC₅₀).

In one aspect of the disclosure, MABEL is determined using a series of test assays run under the conditions that have been optimized as described herein. In one approach, an analysis to determine which assay(s) are most sensitive is undertaken, such that a human dosing regimen is selected in the most conservative (from a safety perspective) manner possible. In some embodiments, MABEL may be determined in an in vitro cell-based assay. In an exemplary embodiment, effector cells are mixed with target cells, and a bispecific antibody such as MEDI-565 is added to the mixture. The effector cells may be T cells and/or peripheral blood mononuclear cells (PBMCs). In some embodiments, the PBMCs may be enriched for CD3+ cells, the cells may be bound by an antibody such as MEDI-565 and activated in order to mediate lysis of CEA-expressing cancer cells. In some embodiments, the PBMCs may be depleted of CD14+ cells, because CD14+ cells such as monocytes may confound results. For example, when monocytes phagocytize dead tumor cells, they become positive for the membrane dye used for target cell labeling. Due to their similar forward scatter (FSC)-side scatter (SSC) appearance, they are difficult to distinguish from the living target cells.

In some embodiments, the target cells may be CEA-expressing cells. Binding of the bispecific antibody may mediate effects that can be quantified, for example, inducing expression of cytokines, increasing expression of T cell activation markers CD69 and CD25, inducing proliferation of PBMCs, increasing T cell lysis and/or killing of the CEA-expressing cells. These effects can be expressed as a function of the bispecific antibody concentration. MABEL may be defined as the effective concentration at which the bispecific antibody induces a minimum effect, for example, 20% maximal effect (EC₂₀).

The conditions for determining MABEL should be optimized. First, target cell lines may be screened and selected. In some embodiments, candidate target cell lines may be any mammalian cell line that has been stably transfected to express CEA, such as CHO cells that express human CEA. In further embodiments, the candidate cells may be mammalian tumor cell lines that naturally express CEA. Examples of CEA-expressing human tumor cell lines include, but are not limited to: A549 (human lung cancer; with mean 7000 CEA cell surface molecules), MKN-45 (human gastric cancer; with mean 165000 CEA cell surface molecules), BxPC3 (human pancreatic cancer; with mean 40000 CEA cell surface molecules) and ASPC-1 (human pancreatic cancer; with mean 90000 CEA cell surface molecules) were tested as candidates for a second target cell line, as they are all human tumor cell lines naturally expressing CEA. Cell lines for MABEL may be tested for their expression of CEA surface molecules, sensitivity to lysis mediated by bispecific antibodies such as BiTE® antibodies (i.e., MEDI-565), efficacy for use with T cell activation assays, and ability to be used for flow cytometry analysis.

In addition, the ratio of effector:target cells (E:T ratio) may be determined. In some embodiments, the ratio is determined by co-incubating a target cell line with increasing concentrations of a bispecific antibody (i.e., MEDI-565) and effector cells. For each cell line, maximal target lysis and minimal donor T cell alloreactivity may be determined. In some embodiments, specific cell lysis is determined by analyzing propidium iodide (PI) incorporation after 48 hours of incubation and/or after 72 hours of incubation. PI is a membrane impermeable dye that is excluded from viable cells, but taken up by dead cells where it can be identified by fluorescent emission, for example, in a flow cytometer. In some embodiments, the expression of T cell markers CD69 and CD25 are measured in each reaction in which increasing concentrations of the bispecific antibody, such as MEDI-565, and effector cells are co-incubated with target cells. In some embodiments, the release of molecules such as one or more cytokines, perforin, and/or granzyme may be measured in each reaction. Exemplary cytokines are IFNγ, TNFα, IL-2, IL-12ₚ₇₀, IL-1β, IL-4, IL-6, IL-8, IL-10, and IL-13.

In further embodiments, the incubation time is optimized. Specific lysis of target cells and/or expression of CD25 and/or CD69 may be measured hourly after at least 1 hour of co-incubation of target cells with effector cells plus a bispecific antibody, such as MEDI-565. Secretion of cytokines may be measured hourly after at least 1 hour of co-incubation of target cells with effector cells plus a bispecific antibody, such as MEDI-565. In some embodiments, the incubation time may be determined by taking measurements at 3 hours, 6 hours, 9 hours, 12 hours, 15 hours, 18 hours, 21 hours, 24 hours, 27 hours, 30 hours, 33 hours, 36 hours, 39 hours, 42 hours, 45 hours, 48 hours, 51 hours, 54 hours, 57 hours, 60 hours, 63 hours, 66 hours, 69 hours, and/or 72 hours.

In some embodiments, where MABEL is calculated from more than one assay, the MABEL values may vary depending on the assay. A very sensitive assay for MABEL is the measure of MEDI-565 that induces T cell lysis of CEA-expressing cells. Accordingly, the EC₂₀ values obtained from this assay may be used as a starting point for determining a therapeutically effective dose for treating human patients.

A MABEL concentration could be identified from an EC₂₀ value (i.e., a concentration that induces 20% of the maximum effect) based on data from in vitro studies (e.g., T-cell activation, cytokine release, cytotoxicity, etc.). Subsequently, pharmacokinetic (PK) modeling in cynomolgus monkeys and an allometric scaling approach may be used to determine a human dose that would result in those exposures (i.e, the equivalent human MABEL concentration). Accordingly, an appropriate dose of the antibody for human administration may be approximated by determination of MABEL of MEDI-565 followed by PK modeling with allometric scaling.

In some embodiments, a dose-escalation scheme is used to determine dose levels at which clinical activity may be observed while maintaining an adequate safety margin. For example, a starting dose of 0.75 μg may be chosen based on the MABEL which was calculated from an EC₂₀ value (i.e., a concentration that induces 20% of the maximum effect) derived from a sensitive in vitro assay (T-cell-mediated cytotoxicity).

An administration schedule may be based on preclinical xenograft studies using exogenous human T cells which showed significant in vivo antitumor activity following daily IV or SC dosing with MEDI-565 for 5 days in mice. The in vitro cytotoxicity assays and PK modeling predict that a 1.5 mg dose, as a three hour IV infusion, of MEDI-565 in humans will achieve blood concentrations equivalent to the EC₅₀ value. In order to achieve these serum concentrations as quickly as possible, while maintaining a sufficient safety margin, an escalation scheme may be used that multiplies each dose by a factor of three until the EC₅₀ dose is reached, at which point a modified Fibonacci escalation scheme is employed.

### III. Dosing, Treatment, and Formulation

A. Dosing

One aspect of the present disclosure relates to methods of treating cancer, comprising administering a specific dose of an antibody comprising a first binding domain that binds to human CD3 and a second binding domain that binds to human CEA, for example the bispecific antibody MEDI-565. Note, however, that any of the antibodies of the disclosure may be used. After MABEL for an antibody, such as MEDI-565, has been determined, a dose may be administered to a patient in need thereof, for use in treating a CEA-expressing cancer. The antibody, such as MEDI-565, is preferably administered to the patient in a therapeutically effective dose that is sufficient to reduce tumor size, volume, growth, metastasis, and/or development of cancer cells in the CEA-expressing cancer, without causing overly toxic effects. For example, a patient suffering from a CEA-expressing cancer receives a therapeutically effective dose of an antibody such as MEDI-565, which comprises a first binding domain that binds to human CEA and a second binding domain that binds to human CD3, wherein the therapeutically effective dose is sufficient to lyse cancer cells and/or trigger an immune response against the cancer cells. In some embodiments, the dose is sufficient to lyse at least 20%, 30%, 40%, 45%, 50%, or at least 60% of the cells that express CEA. In further embodiments, the dose is sufficient to increase the release of one or more molecules associated with activation of the immune response, for example, pro-inflammatory cytokines, perforin, and/or granzyme by at least 30%, 40%, 45%, or at least 50% relative to untreated cells. Exemplary pro-inflammatory cytokines include IFNγ, TNFα, IL-2, IL-12ₚ₇₀, IL-1β, IL-4, IL-6, IL-8, IL-10, and IL-13. In some embodiments, a therapeutically effective dose of an antibody such as MEDI-565 may be sufficient to reduce tumor volume by at least 25%, as compared to untreated control tumors. A therapeutically effective dose may also be sufficient to increase expression of T cell activation markers CD69 and CD25 by at least 25%, relative to untreated cells. In addition, a therapeutically effective dose may induce proliferation of peripheral blood mononuclear cells. In certain embodiments, the method further comprises obtaining a sample from the patient and assaying any one or more of these markers, such as cytokine expression or expression of T cell activation markers. In certain embodiments, the patient sample is obtained before, after, or during administration of the antibody. An assay may also comprise in vivo imaging of a patient, such as magnetic resonance imaging (MRI) or positron emission tomography (PET) scan to evaluate tumor volume, number of tumors, and/or spread of tumors.

Any cancer that expresses CEA is a candidate for treatment according to the methods and dosing regimens described herein. Exemplary CEA-expressing cancers include, but are not limited to, colon cancer, colorectal cancer, ovarian cancer, prostate cancer, rectal cancer, pancreatic cancer, esophageal cancer, gastroesophageal cancer, stomach cancer, lung cancer, and breast cancer. For example, the cancer may be an adenocarcinoma of gastrointestinal origin. In some embodiments, the cancer is a relapsed or refractory cancer. For example, the cancer may be a refractory pancreatic adenocarcinoma or a refractory colorectal cancer (CRC).

A dose may refer to a specific quantity of an antibody therapeutic, such as MEDI-565, which may be taken at any one time or at specified intervals. The term “dosing”, as used herein, refers to the administration of a substance (e.g., a bispecific antibody such as MEDI-565), or a pharmaceutical composition comprising same, to achieve a therapeutic objective (e.g., the treatment of a CEA-expressing cancer). Thus, a dosing schedule is a combination of the dose and the time intervals at which the dose is administered. For example, a dosing schedule may comprise administering a dose of 0.75 μg per day of a bispecific antibody such as MEDI-565, once per day for at least one day. In some embodiments, the dosing schedule comprises administering a bispecific antibody such as MEDI-565 in an amount and at an interval sufficient to maintain a desired serum concentration of the bispecific antibody for a desired time period.

Serum concentrations of the antibody may be monitored over time. One aspect of the disclosure relates to a method for treating a CEA-expressing cancer, comprising administering to a human patient in need of treatment a protein composition, which protein composition comprises an antibody comprising a first binding domain that binds to human CEA and a second binding domain that binds to human CD3, wherein the protein composition is administered on a dosing schedule and at a dose of antibody that maintains a serum concentration of the antibody below 0.097 ng/mL, such as about 0.1 ng/ml. In some embodiments, the dose of antibody maintains a serum concentration of the antibody of at least about 2 ng/mL. These serum concentrations are based on the EC₂₀ and EC₅₀, respectively, obtained from an in vitro assay for T-cell mediated lysis of CEA-expressing cells, a sensitive measure for MABEL.

Another aspect of the disclosure relates to a method for treating a CEA-expressing cancer, comprising administering a protein composition, which protein composition comprises an antibody comprising a first binding domain that binds to human CEA and a second binding domain that binds to human CD3, which antibody is provided at a dose of 0.75 μg to 10 mg per day on a dosing schedule comprising administering the protein composition once per day for at least one day.

In some embodiments, a dosing schedule is part of a treatment cycle of 21 days. In some embodiments, a dosing schedule is part of a treatment cycle of 28 days. The term “treatment cycle”, as used herein, refers to the period wherein the antibody is administered followed by a period with no administration of the antibody. The beginning of the next cycle is marked by the re-initiation of administration of the antibody. Thus, treatment cycles allow for a period of rest between days of administration of antibody. A treatment cycle may vary in number of days, for example, 15, 16, 17, 18, 19, 20, 21, 22, 23, 24, 25, 26, 27, 28, 29, or 30 days. Moreover, the total number of treatment cycles used may be selected by the physician based on the patient's condition, extent of disease, age, and responsiveness to therapy.

The antibody may be any of the antibodies described herein, such as the bispecific antibody MEDI-565. For example, the antibody may be provided at a dose per day ranging from 0.75 μg to 2.25 μg, 2.25 μg to 6.75 μg, 6.75 μg to 20 μg, 20 μg to 60 μg, 60 μg to 180 μg, 180 μg to 540 μg, 540 μg to 1.5 mg, 1.5 mg to 3 mg, 3 mg to 5 mg, 5 mg to 7.5 mg, 7.5 mg to 10 mg. In some embodiments, the antibody may be provided at a dose per day of 1.5 mg. In further embodiments, the antibody may be provided at a dose per day of 0.75 μg, 2.25 μg, 6.75 μg, 20 μg, 60 μg, 180 μg, 540 μg, 1.5 mg, 3 mg, 5 mg, 7.5 mg, or 10 mg. In certain embodiments, the antibody may be provided at a dose per day of greater than 10 mg, such as 15 mg, 20 mg, 25, mg, 30 mg, 40 mg, 45 mg, 50 mg, 60 mg, 70 mg, 75 mg, 80 mg, 85 mg, 90 mg, 95 mg, or 100 mg.

In some embodiments, the dose is calculated based on in vitro potency, and corresponds to a concentration of the antibody that produced a target effect in an in vitro assay, for example, a 50% maximal effect (EC₅₀) or a 20% maximal effect (EC₂₀). In an exemplary embodiment, a dose of about 1.5 mg of MEDI-565 is administered by IV for three hours. In another embodiment, a dose of about 0.75 μg of MEDI-565 is administered by IV for three hours. Such doses are based on combing in vitro studies with PK data in monkeys, followed by allometric scaling to predict human PK parameters.

In some embodiments, the antibody comprising a first binding domain that binds to human CEA and a second binding domain that binds to human CD3 (i.e., MEDI-565), is administered on more than one day. For example, the antibody may be administered once per day for 1, 2, 3, 4, 5, 6, 7, 8, 9, or 10 days. In some embodiments, the antibody may be administered once per day for at least 3 consecutive days. In other embodiments, the antibody is administered once per day for five consecutive days. The same dose of the antibody may be administered on each day of administration, or a different dose of the antibody may be administered on each day of administration. For example, a patient may receive a higher dose of antibody on a day of administration, relative to the dose received on a previous day of administration. Or, a patient may receive a lower dose of antibody on a day of administration, relative to the dose received on a previous day of administration.

Administration of an antibody such as the bispecific antibody MEDI-565 may occur over one or more additional treatment cycles. Thus, the same dosing schedule may be repeated again after a first treatment cycle is completed. For example, an antibody may be administered on a dosing schedule comprising administering the antibody once per day for at least one day within a first treatment cycle of 21 days. As another example, an antibody may be administered on a dosing schedule comprising administering the antibody once per day for at least one day within a first treatment cycle of 21 or 28 days. Then the antibody may be administered again on a dosing schedule comprising the antibody once per day for at least one day within a second treatment cycle of 21 or 28 days. In some embodiments, the antibody may be administered once per day for 1, 2, 3, 4, 5, 6, 7, 8, 9, or 10 days within a first treatment cycle. In some embodiments, the antibody may be administered once per day for at least 3 consecutive days within a first treatment cycle. In other embodiments, the antibody is administered once per day for five consecutive days within a first treatment cycle. During a second treatment cycle and/or any subsequent treatment cycles, the antibody may again be administered for once per day for at least one day, for example, or once per day for 1, 2, 3, 4, 5, 6, 7, 8, 9, and/or 10 days. In any given treatment cycle, the antibody may be administered once per day for the same number of days as it is administered in other treatment cycles. Alternatively, the number of days during which the antibody is administered may vary across treatment cycles. Similarly, the length of the treatment cycle may vary. A shorter treatment cycle represents fewer “rest days” prior to re-initiation of administration of antibody, whereas a longer treatment cycle represents additional rest days. Of course, the exact number of rest days depends not only on the length of the treatment cycle but also on the number of days of antibody administration.

Accordingly, the dose of an antibody administered during each treatment cycle may be varied relative to other treatment cycles by varying the number of days during which the antibody is administered. In some embodiments, where the number of days of administration is similar across all treatment cycles, the dose of the antibody may be increased in a second (and/or subsequent) treatment cycle, as compared with the first treatment cycle. For example, the dose of the antibody in the second treatment cycle may be threefold that of the first treatment cycle.

In some embodiments, the antibody is administered intravenously (IV). For example, the antibody may be administered by IV infusion over a period of 3 hours per day (or over a period of less than 3 hours or more than 3 hours). Administration by, for example, infusion over a period of time (e.g., 1, 2, 3 hours) is considered one administration, such that one three hour infusion in a day is considered administration once per day.

B. Treatment

A further aspect of the disclosure relates to methods for treating CEA-expressing cancers, comprising administering to a patient in need of treatment a protein composition, which protein composition comprises an antibody comprising a first binding domain that binds to human CEA and a second binding domain that binds to human CD3 (e.g., MEDI-565), which antibody is provided at a dose of 0.75 μg to 10 mg per day (or more) on a dosing schedule comprising administering the protein composition once per day for at least one day. In some embodiments, the dose of antibody such as MEDI-565 is administered on a dosing schedule sufficient to maintain a serum concentration of the antibody at about a target level, for example, a serum concentration between 0.097 ng/mL and about 2 ng/mL, such as between about 0.1 ng/mL and about 2 ng/mL. In some embodiments, a dose of antibody such as MEDI-565 is administered on a dosing schedule sufficient to maintain a serum concentration of the antibody above about 2 ng/ml. In some embodiments, a dose of antibody such as MEDI-565 is administered on a dosing schedule sufficient to maintain a serum concentration of the antibody above about 4 ng/ml. In some embodiments, a dose of antibody such as MEDI-565 is administered on a dosing schedule sufficient to maintain a serum concentration of the antibody above about 6.7 ng/ml. In some embodiments, a dose of antibody such as MEDI-565 is administered on a dosing schedule sufficient to maintain a serum concentration of the antibody above about 10 ng/ml. In some embodiments, a dose of antibody such as MEDI-565 is administered on a dosing schedule sufficient to maintain a serum concentration of the antibody above about 13.3 ng/ml.

For example, a patient diagnosed with a CEA-expressing cancer may be treated according to a method in which an antibody such as the bispecific antibody MEDI-565 is provided at a dose per day of 0.75 μg to 10 mg. In other embodiments, the dose per day is greater than 10 mg, such as 20, 30, 50, 70, 80 or 100 mg. The dose may be administered intravenously, over a period of at least one hour, for example, over a period of 3 hours, once per day. The patient may receive an IV dose once per day for more than one consecutive day, for example, for 5 consecutive days. In some embodiments, the methods comprise dosing according to a treatment cycle of 21 or 28 days, so that a patient receives a dose once per day for at least 3 days, for example, for 5 days, and then does not receive treatment again during the 21- or 28-day cycle. In further embodiments, the method comprises one or more treatment cycles, during which the patient receives additional doses of the antibody. In some embodiments, the initial dose of the antibody (e.g., MEDI-565) may be 0.75 μg, 2.25 μg, 6.75 μg, 20 μg, 60 μg, 180 μg, 540 μg, 1.5 mg, 3 mg, 5 mg, 7.5 mg, or 10 mg, (or more than 10 mg) and may be administered once per day by intravenous infusion over a period of 3 hours, for 5 consecutive days during the first treatment cycle. During subsequent treatment cycles, the dose may be the same, or may be decreased or increased.

In some embodiments, patients may be in need of treatment of colon cancer, ovarian cancer, prostate cancer, rectal cancer, pancreatic cancer, esophageal cancer, stomach cancer, lung cancer, and/or breast cancer. For example, the patients may be in need of treatment of adenocarcinoma of gastrointestinal origin. In some embodiments, the cancer is a relapsed or refractory cancer. For example, the cancer may be a refractory pancreatic adenocarcinoma or a refractory colorectal cancer (CRC).

During the course of treatment, patient data may be collected and used to assess the efficacy of treatment. Relevant data include PK data (for example, PK data parameters comprise the bioavailability of the antibody, as determined by plotting serum concentration as a function of time and determining the area under the serum concentration time curve (area under curve, or AUC); steady state concentration; maximum concentration (Cmax); time to reach maximum concentration (Tmax); clearance of the antibody (CL); volume of distribution (Vd), serum half life of the antibody (t1/2)), pharmacodynamic data, biomarker data, and anti-tumor activity data. In some embodiments, peripheral blood cell populations (such as T cells, subsets of T cells, NK cells, and/or B cells) are quantified. In some embodiments, the cytokine response is measured. Finally, the tumor may be examined according to the Response Evaluation Criteria In Solid Tumors (RECIST) guidelines (Eisenhauer et al.). The patients may be assigned to one of the following categories: complete response, partial response, stable disease, progression, or inevaluable.

A change in the measured parameters may indicate that the patient has had a therapeutic response. For example, between a first time point and a second time point, a reduction may be observed in the size, volume, growth, metastasis, and/or development of cancer cells in the CEA-expressing cancer, expression of biomarkers, and/or expression of molecules associated with an immune response. CD69 and CD25 upregulation on T cells and specific lysis of CEA-expressing cancer cells, as described herein, are indicators for biological activity of an antibody such as MEDI-565. Immune cells may be collected from a patient's blood and analyzed for expression of markers or quantified to determine proliferation. Similarly, increased lysis of cells that express CEA; increased release of one or more pro-inflammatory cytokines, perforin and/or granzyme; increased T cell activation; and increased proliferation of peripheral blood mononuclear cells (particularly, CD3+ T cells) may be observed. The first time point may be prior to administration of the antibody, or may be after the first day of administration of the antibody. In some embodiments, the first time point is at the beginning of a 28 day treatment cycle. The second time point may be subsequent to administration of the antibody, for example, at the end of a 28 day treatment cycle. It is understood that whether a dose is therapeutically effective may not be observable after only a single dose. However, a dose that is effective over either a single administration or multiple administrations is considered therapeutically effective.

In some embodiments, when no measurable and/or significant change has been measured, the treatment with an antibody such as MEDI-565 should be continued. For example, a larger dose of the antibody may be administered, and/or additional treatment cycles may be added.

C. Formulation

In some embodiments, the protein composition comprising an antibody comprising a first binding domain that binds to human CEA and a second binding domain that binds to human CD3 (e.g. MEDI-565) is formulated for intravenous administration. An exemplary formulation may be reconstituted from a sterile lyophilized formulation, for example, suitable amount of MEDI-565 may be contained in a vial. The formulation after reconstitution may be a suitable concentration in suitable buffer containing, for example, salts, buffer, saccharides and/or polyols, and surfactant.

Following dilution into the final IV bag, MEDI-565 drug product is administered as an IV infusion over about ½, ¾, 1, 1.5, 2, 2.5, 3, or even greater than 3 hours.

### EXEMPLARY EMBODIMENTS

1. A method for treating a CEA-expressing cancer, comprising administering to a human patient in need of treatment a protein composition, which protein composition comprises an antibody comprising a first binding domain that binds to human CD3 and a second binding domain that binds to human CEA, which antibody is provided at a dose of 0.75 μg to 10 mg per day on a dosing schedule comprising administering the protein composition once per day for at least one day.
2. The method of embodiment 1, wherein the dosing schedule is part of a treatment cycle of 21 or 28 days.
3. The method of embodiment 1 or 2, wherein the CEA-expressing cancer is chosen from: colon cancer, ovarian cancer, prostate cancer, rectal cancer, pancreatic cancer, esophageal cancer, stomach cancer, lung cancer and breast cancer.
4. The method of embodiment 3, wherein the CEA-expressing cancer is a relapsed or refractory cancer.
5. The method of any of embodiments 1-4, wherein the CEA-expressing cancer is an adenocarcinoma of gastrointestinal origin.
6. The method of any of embodiments 1-5, wherein the antibody is a bispecific single chain antibody comprising a first binding domain that binds to human CD3 and a second binding domain that binds to the human CEA.
7. The method of embodiment 6, wherein the bispecific single chain antibody comprises an amino acid sequence chosen from the amino acid sequences of SEQ ID NOs: 28-44 and 46-52.
8. The method of embodiment 6, wherein the bispecific single chain antibody comprises the amino acid sequence of SEQ ID NO: 48.
9. The method of embodiment 6, wherein the bispecific single chain antibody comprises the amino acid sequence of SEQ ID NO: 49.
10. The method of embodiment 6, wherein the bispecific single chain antibody comprises the amino acid sequence of SEQ ID NO: 46.
11. The method of embodiment 6, wherein the bispecific single chain antibody comprises the amino acid sequence of SEQ ID NO: 51.
12. The method of any of embodiments 1-11, wherein the antibody is provided at a dose of 0.75 μg to 2.25 μg per day.
13. The method of any of embodiments 1-11, wherein the antibody is provided at a dose of 2.25 μg to 6.75 μg per day.
14. The method of any of embodiments 1-11, wherein the antibody is provided at a dose of 6.75 μg to 20 μg per day.
15. The method of any of embodiments 1-11, wherein the antibody is provided at a dose of 20 μg to 60 μg per day.
16. The method of any of embodiments 1-11, wherein the antibody is provided at a dose of 60 μg to 180 μg per day.
17. The method of any of embodiments 1-11, wherein the antibody is provided at a dose of 180 μg to 540 μg per day.
18. The method of any of embodiments 1-11, wherein the antibody is provided at a dose of 540 μg to 1.5 mg per day.
19. The method of any of embodiments 1-11, wherein the antibody is provided at a dose of 1.5 mg to 3 mg per day.
20. The method of any of embodiments 1-11, wherein the antibody is provided at a dose of 1.5 mg per day.
21. The method of any of embodiments 1-11, wherein the antibody is provided at a dose of 3 mg to 5 mg per day.
22. The method of any of embodiments 1-11, wherein the antibody is provided at a dose of 5 mg to 7.5 mg per day.
23. The method of any of embodiments 1-11, wherein the antibody is provided at a dose of 7.5 mg to 10 mg per day.
24. The method of any of embodiments 1-23, wherein the protein composition is administered intravenously.
25. The method of any of embodiments 1-24, wherein the protein composition is administered by intravenous infusion over a period of 3 hours per day.
26. The method of any of embodiments 1-25, wherein the protein composition is administered on a dosing schedule comprising administering the protein composition once per day for at least 3 consecutive days.
27. The method of embodiment 26, wherein the protein composition is administered on a dosing schedule comprising administering the protein composition once per day for five consecutive days.
28. The method of any of embodiments 1-27, further comprising one or more additional treatment cycles of 28 days, wherein the protein composition is administered on a dosing schedule comprising administering the protein composition once per day for at least one day per each treatment cycle.
29. The method of embodiment 28, wherein the protein composition is administered on a dosing schedule comprising administering the protein composition once per day for at least 3 consecutive days per each treatment cycle.
30. The method of embodiment 29, wherein the protein composition is administered on a dosing schedule comprising administering the protein composition once per day for 5 consecutive days per each treatment cycle.
31. The method of any of embodiments 28-30, wherein the patient is administrated the same dose of the antibody in the protein composition each day of administration.
32. The method of any of embodiments 28-30, wherein patient is administered an increasing dose of the antibody in the protein composition during a second treatment cycle relative to a first treatment cycle.
33. The method of embodiment 32, wherein the dose of antibody in the protein composition during the second treatment cycle is three fold that during the first treatment cycle.
34. The method of any of embodiments 28-30, wherein the patient is administered a higher dose of the antibody in the protein composition on a day of administration relative to the dose on a previous day of administration.
35. The method of any of embodiments 28-30, wherein the patient is administered a lower dose of the antibody in the protein composition on a day of administration relative to the dose on a previous day of administration.
36. The method of any of embodiments 1-35, wherein the patient receives a therapeutically effective dose sufficient to lyse at least about 20%, 25%, 30%, 35%, 40%, 45%, 50%, 55%, 60% of the cancerous cells that express CEA.
37. The method of any of embodiments 1-36, wherein the patient receives a therapeutically effective dose sufficient to increase release of one or more pro-inflammatory cytokines, perforin, and/or granzyme by at least about 50% relative to untreated cells.
38. The method of embodiment 37, wherein the one or more proinflammatory cytokines are chosen from IFNγ, TNFα, IL-2, IL-12ₚ₇₀, IL-10, IL-4, IL-6, IL-8, IL-10, and IL-13.
39. The method of any of embodiments 1-38, wherein the patient receives a therapeutically effective dose sufficient to reduce tumor volume by at least about 25%, as compared to untreated control tumors.
40. The method of any of embodiments 1-39, wherein the patient receives a therapeutically effective dose sufficient to increase expression of T cell activation markers CD69 and CD25 by at least about 25%, relative to untreated cells.
41. The method of any of embodiments 1-40, wherein the patient receives a therapeutically effective dose sufficient to induce proliferation of CD3+ T cells of peripheral blood mononuclear cells.
42. The method of any of embodiments 1-41, further comprising measuring therapeutic efficacy, wherein a measured change in the patient between an earlier time point and a subsequent time point indicates that the protein composition is therapeutically effective.
43. The method of embodiment 42, wherein the measured change is chosen from at least one of increased lysis of cells that express CEA; increased release of one or more pro-inflammatory cytokines, perforin and/or granzyme; decreased tumor volume; increased T cell activation; and increased proliferation of peripheral blood mononuclear cells.
44. The method of embodiment 42 or 43, wherein the first time point is prior to administration of the protein composition.
45. The methods of any of embodiments 42-44, wherein the first time point is after the first day of administration of the protein composition.
46. The methods of any of embodiments 42-45, wherein the first time point is at the beginning of a 28 day treatment cycle.
47. The method of embodiment 46, wherein the second time point is at the end of a 28 day treatment cycle.
48. The method of any of embodiments 1-47, wherein the dosing schedule maintains the antibody at a serum concentration between about 0.1 ng/mL to about 2 ng/mL in the patient for at least 4 hours.
49. The method of any of embodiments 1-47, and 50-66, wherein the dosing schedule maintains the antibody at a serum concentration between greater than about 2 ng/ml, greater than about 4 ng/ml, greater than about 6.7 ng/ml, greater than about 10 ng/ml, or greater than about 13.3 ng/ml, in the patient for at least 4 hours, for at least 24 hours, or for at least 1 week.
50. A method for treating a CEA-expressing cancer, comprising administering to a human patient in need of treatment a protein composition, which protein composition comprises an antibody comprising a first binding domain that binds to human CD3 and a second binding domain that binds to human CEA, wherein the protein composition is administered on a dosing schedule and at a dose of antibody that maintains a serum concentration of the antibody of at least about 0.1 ng/mL.
51. The method of embodiment 50, wherein the antibody comprises an amino acid sequence chosen from the amino acid sequences of SEQ ID NOs: 28-44 and 46-52.
52. The method of embodiment 51, wherein the antibody comprises the amino acid sequence of SEQ ID NO: 48.
53. The method of embodiment 51, wherein the antibody comprises the amino acid sequence of SEQ ID NO: 49.
54. The method of embodiment 51, wherein the antibody comprises the amino acid sequence of SEQ ID NO: 46.
55. The method of embodiment 51, wherein the antibody comprises the amino acid sequence of SEQ ID NO: 51.
56. The method of any of embodiments 50-55, wherein the dosing schedule comprises administering the protein composition to the patient once per day for at least one day.
57. The method of embodiment 56, wherein the dosing schedule occurs during a treatment cycle of 28 days.
58. The method of any of embodiments 50-57, wherein the CEA-expressing cancer is chosen from: colon cancer, ovarian cancer, prostate cancer, rectal cancer, pancreatic cancer, esophageal cancer, stomach cancer, lung cancer and breast cancer.
59. The method of any of embodiments 50-58, wherein the CEA-expressing cancer is a relapsed or refractory cancer.
60. The method of any of embodiments 50-59, wherein the CEA-expressing cancer is an adenocarcinoma of gastrointestinal origin.
61. The method of any of embodiments 50-60, wherein the antibody is a bispecific single chain antibody comprising a first binding domain that binds to human CEA and a second binding domain that binds to the human CD3.
62. The method of any of embodiments 50-61, wherein the protein composition is administered intravenously.
63. The method of any of any of embodiments 50-62, wherein the protein composition is administered by intravenous infusion over a period of 3 hours per day.
64. The method of any of any of embodiments 50-63, wherein the protein composition is administered on dosing schedule comprising administering the protein composition once per day for at least 3 consecutive days.
65. The method of embodiment 64, wherein the protein composition is administered on a dosing schedule comprising administering the protein composition once per day for five consecutive days.
66. The method of any of embodiments 50-65, further comprising one or more additional treatment cycles of 28 days.
67. A method of treating a CEA-expressing cancer, comprising administering to a patient in need thereof a composition comprising an antibody comprising a first binding domain that binds to human CD3 and a second binding domain that binds to human CEA at a dose of antibody and on a dosing schedule sufficient to maintain a serum concentration of antibody that that is therapeutically effective and sufficient to lyse at least about 60% of the cancerous cells that express CEA.
68. A protein composition comprising an antibody comprising a first binding domain that binds to human CD3 and a second binding domain that binds to human CEA for use in treating a CEA-expressing cancer, wherein the antibody is administered at a dose of about 0.75 μg to about 10 mg per day on a dosing schedule comprising administering the protein composition once per day for at least one day.
69. A protein composition comprising an antibody comprising a first binding domain that binds to human CD3 and a second binding domain that binds to human CEA for use in treating a CEA-expressing cancer by administration of about 0.75 μg to about 10 mg of antibody per day on a dosing schedule in which the protein composition is administered once per day for at least one day.
70. The protein composition of embodiment 68 or 69, wherein the dosing schedule is part of a treatment cycle of 28 days.
71. The protein composition of any of embodiments 68-70, wherein the antibody comprises a bispecific single chain antibody comprising an amino acid sequence chosen from the amino acid sequences of SEQ ID NOs: 28-44 and 46-51.
72. The protein composition of any of embodiments 68-71, wherein the antibody is provided at a dose of 540 μg to 1.5 mg per day.
73. The protein composition of any of embodiments 68-71, wherein the antibody is provided at a dose of 1.5 mg to 3 mg per day.
74. The protein composition of any of embodiments 68-73, wherein the antibody is provided at a dose of 1.5 mg per day.
75. The protein composition of any of embodiments 68-71, wherein the antibody is provided at a dose of 3 mg to 7.5 mg per day.
76. The protein composition of any of embodiments 68-71, wherein the antibody is provided at a dose of 5 mg to 10 mg per day.
77. The protein composition of any of embodiments 68-76, wherein the protein composition is administered by intravenous infusion over a period of 3 hours per day.
78. The protein composition of any of embodiments 68-77, wherein the protein composition is administered on a dosing schedule comprising administering the protein composition once per day for at least 3 consecutive days.
79. The protein composition of any of embodiments 68-78, wherein the protein composition is administered on a dosing schedule comprising administering the protein composition once per day for five consecutive days.
80. The protein composition of any of embodiments 68-79, wherein the dosing schedule maintains the protein composition at a serum concentration between about 0.1 ng/mL to about 2 ng/mL, or greater than about 2 ng/mL, in the patient for at least 4 hours.
81. A protein composition comprising an antibody comprising a first binding domain that binds to human CD3 and a second binding domain that binds to human CEA for use in treating a CEA-expressing cancer, wherein the protein composition is administered on a dosing schedule and at a dose of antibody that maintains a serum concentration of the protein composition of at least about 0.1 ng/mL.
82. An antibody comprising a first binding domain that binds to human CD3 and a second binding domain that binds to human CEA for use in treating a CEA-expressing cancer, wherein the antibody is administered at a dose of about 0.75 μg to about 10 mg per day on a dosing schedule comprising administering the antibody once per day for at least one day.
83. An antibody comprising a first binding domain that binds to human CD3 and a second binding domain that binds to human CEA for use in treating a CEA-expressing cancer by administration of about 0.75 μg to about 10 mg of antibody per day on a dosing schedule in which the antibody is administered once per day for at least one day.
84. The method of any of embodiments 1-67, wherein the antibody comprises an amino acid sequence chosen from SEQ ID NOs: 28-44, and 46-52.
85. The method of any of embodiments 1-67 and 84, wherein the antibody comprises an amino acid sequence chosen from SEQ ID NOs: 36, 37, 41-43, and 47.
86. The method of any of embodiments 1-67, 84, and 85, wherein the antibody comprises the sequence of SEQ ID NO: 52.
87. The protein composition of any of embodiments 68-81, wherein the antibody comprises an amino acid sequence chosen from SEQ ID NOs: 28-44, and 46-52.
88. The protein composition of any of embodiments 68-81 and 87, wherein the antibody comprises an amino acid sequence chosen from SEQ ID NOs: 36, 37, 41-43, and 47.
89. The protein composition of any of embodiments 68-81, 87, and 88, wherein the antibody comprises the sequence of SEQ ID NO: 52.

### EXAMPLES

Having generally described the disclosure, Applicants refer to the following illustrative examples to help to understand the generally described disclosure. These specific examples are included merely to illustrate certain aspects and embodiments of the present disclosure, and they are not intended to limit the disclosure in any respect. Certain general principles described in the examples, however, may be generally applicable to other aspects or embodiments of the disclosure. The disclosure contemplates that any one or more of the aspects, embodiments and other features described above and below can be combined.

### Example 1

### Assay Development

A variety of CEA-expressing cell lines were tested as potential target cell populations for MABEL analysis in fluorescence activated cell sorting (FACS)-based cytotoxicity and T cell activation assays. Dhfr-CHO, MKN45, ASPC-1, BxPC3, and A549 cell lines were all tested. Two cell lines were selected as target cell lines for further assay development: CHO/huCEA, which are CHO cells that have been engineered to express high numbers of CEA cell surface molecules (340000±180000), and ASPC-1 cells (a human pancreatic cancer cell line). CHO/huCEA cells are sensitive to MEDI-565-induced redirected T cell lysis, are efficacious when used in T cell activation assays, and can be subjected to flow cytometry analysis. ASPC-1 cells are a human tumor cell line that naturally expresses CEA (about 90000 CEA cell surface molecules).

A. Determining E:T Ratios and Incubation Times for T Cell Lysis of CHO/huCEA Cells, T Cell Expression of CD69 and CD25, and Release of Cytokines

The ratio of effector cells to target cells (E:T ratio) influences the bioactivity of MEDI-565. Thus, various E:T ratios were analyzed to establish the most sensitive and reliable assay setup. As described above, target cells which express CEA were used, and effector cells may include populations of peripheral blood mononuclear cells (PBMCs) in which CD3+ cells have been enriched or CD14+ cells have been depleted. PBMCs depleted of CD14+ are referenced herein as PBMC without CD14 and PBMC enriched for CD3+ cells are referenced herein as PBMC with CD3ε.

Target cells (CHO/huCEA cells) and effector cells (PBMC with CD3ε) were co-cultured in ratios ranging from 1:2 to 80:1 with increasing concentrations of MEDI-565 for 72 hours. For technical reasons, in one setting, 10000 target cells were combined with varying amounts of effector cells (E:T from 1:2 to 20:1), while in another setting a constant amount of effector cells (100000) was combined with varying numbers of target cells (E:T of 80:1). Specific cell lysis was determined by analysis of propidium iodide (PI) incorporation, and T cell activation was determined by de novo expression of the surface markers CD69 or CD25.

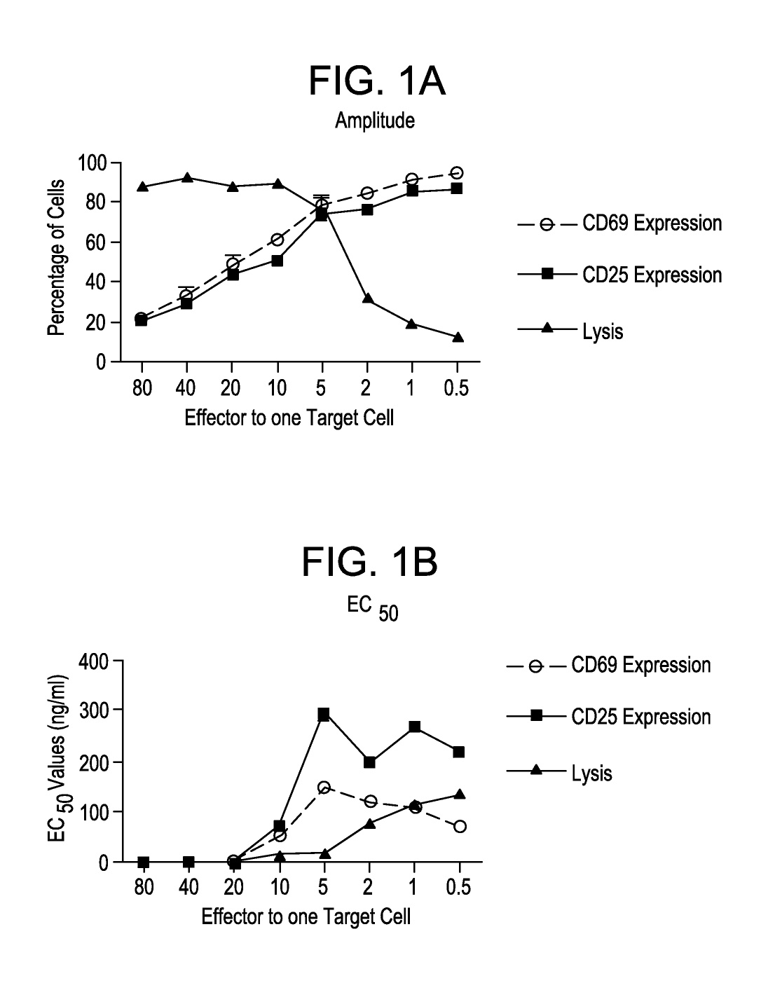

At E:T ratios of 10:1 and higher, maximal lysis was more or less constant, but declined steadily at E:T cell ratios of 5:1 and less. Conversely, the percentage of CD25+ and CD69+ T cells gradually decreased with increasing E:T ratio (FIG. 1A). Thus, the most reliable assay setting using CHO/huCEA cells and PBMCs with CD3ε was an E:T ratio of 5:1. EC₅₀ values were inversely proportional to the E:T cell ratio; lower E:T cell ratios achieved higher EC₅₀ values, and accordingly, the most sensitive assay system would use an E:T ratio higher than 10:1 (FIG. 1B).

Thus, an E:T cell ratio of 10:1 was chosen as optimal for further experiments using PBMCs with CD3ε to obtain high sensitivity and reliable assay conditions.

Additionally, the incubation time was optimized. To analyze the kinetics of T cell activation mediated by MEDI-565, PBMC with CD3ε or without CD14 were co-cultured with CHO/huCEA cells at an E:T cell ratio of 10:1 in the presence of 10 μg/mL MEDI-565 or control BiTE® antibody for 72 hours.

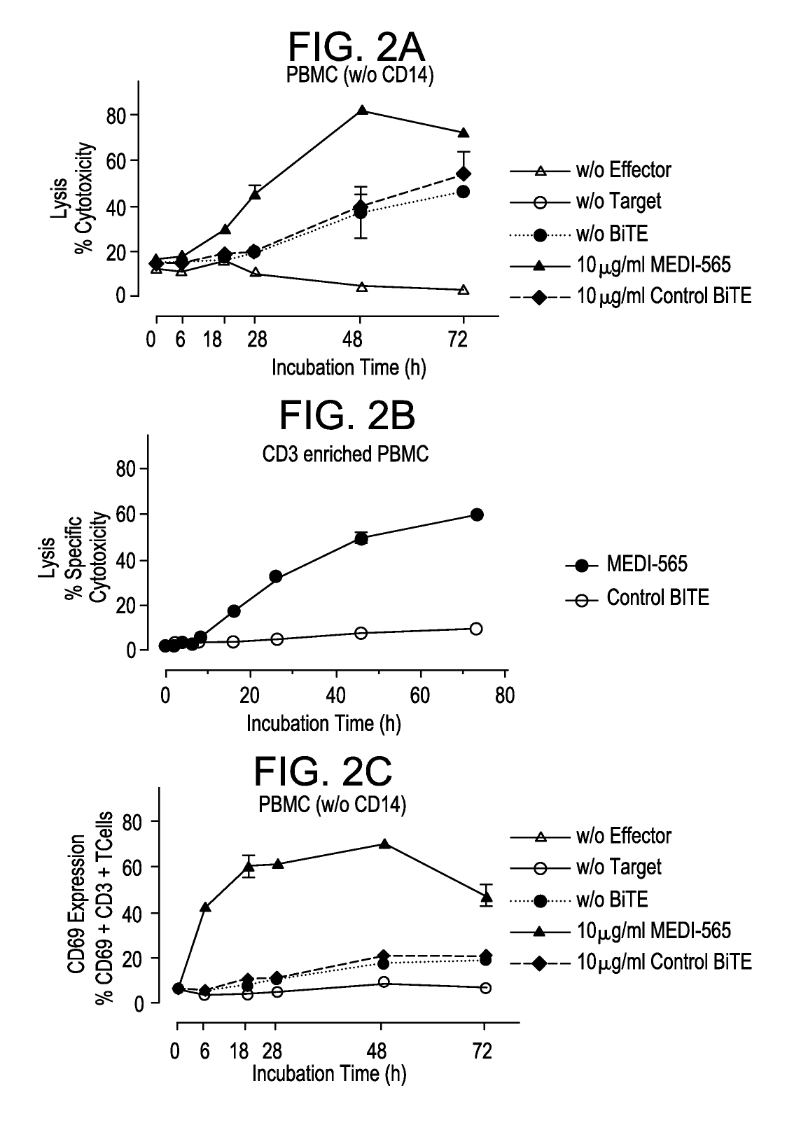

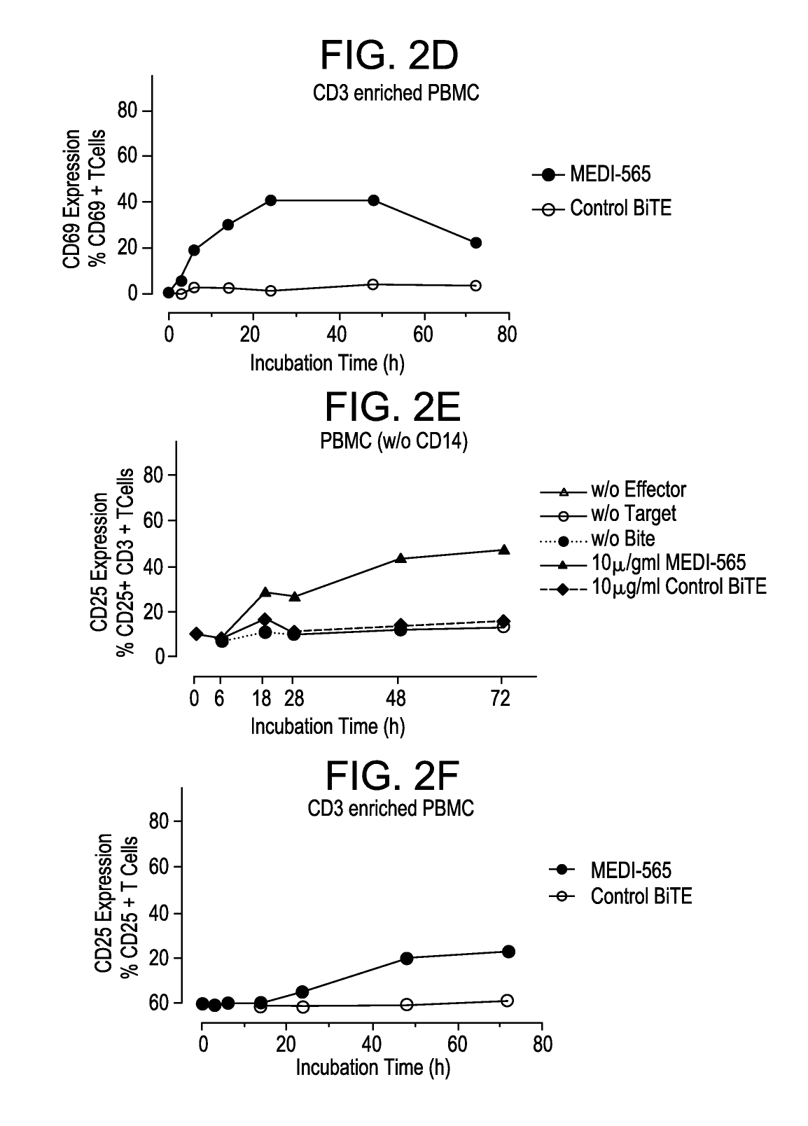

Specific lysis of CHO/huCEA cells increased over time and reached a maximum of about 80% and 60% in cultures containing PBMC without CD14 (FIG. 2A) and with CD3ε (FIG. 2B), respectively, by 72 hours. Time points later than 72 hours were not tested due to limitations of the assay settings, ie, proliferating target cells would have obscured results after longer incubation periods. As shown in FIGS. 2C and D, MEDI-565 induced upregulation on T cells of CD69, which could be detected at the earliest analyzed time point (3 hours). After 24 hours, 40% to 60% of all T cells were activated as indicated by CD69 expression. The number of CD69 positive T cells started to decline after 48 hours. CD25 was only upregulated on approximately 25% to 40% of T cells and expression started later (first measurable after 14 hours) than that of CD69 (FIG. 2E, F). Over the time course measured, no decline of CD25 expression on the T cells was detected. Thus, an incubation time of 72 hours was chosen as optimal for the MABEL determination in order to obtain the best response with the highest assay sensitivity.

To analyze the kinetics of cytokine secretion induced by MEDI-565, CHO/huCEA cells were co-cultured with PBMCs without CD14 at an E:T cell ratio of 10:1 either alone or in the presence of 10 μg/mL MEDI-565 for up to 72 hours.

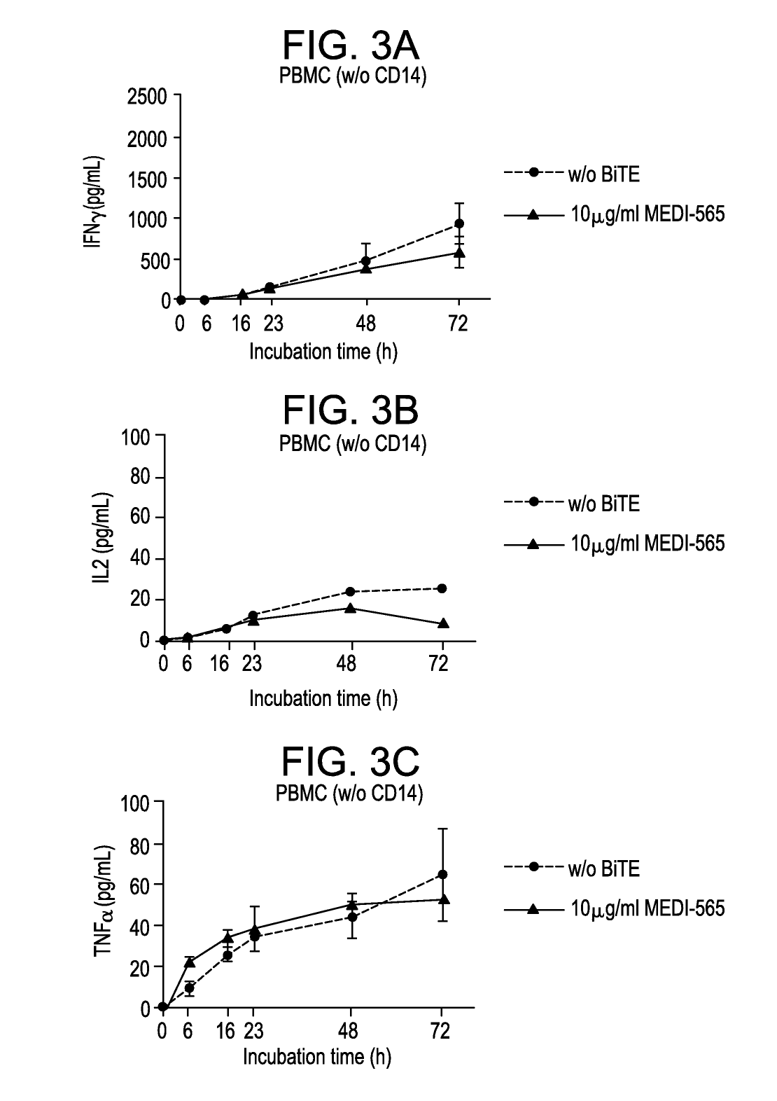

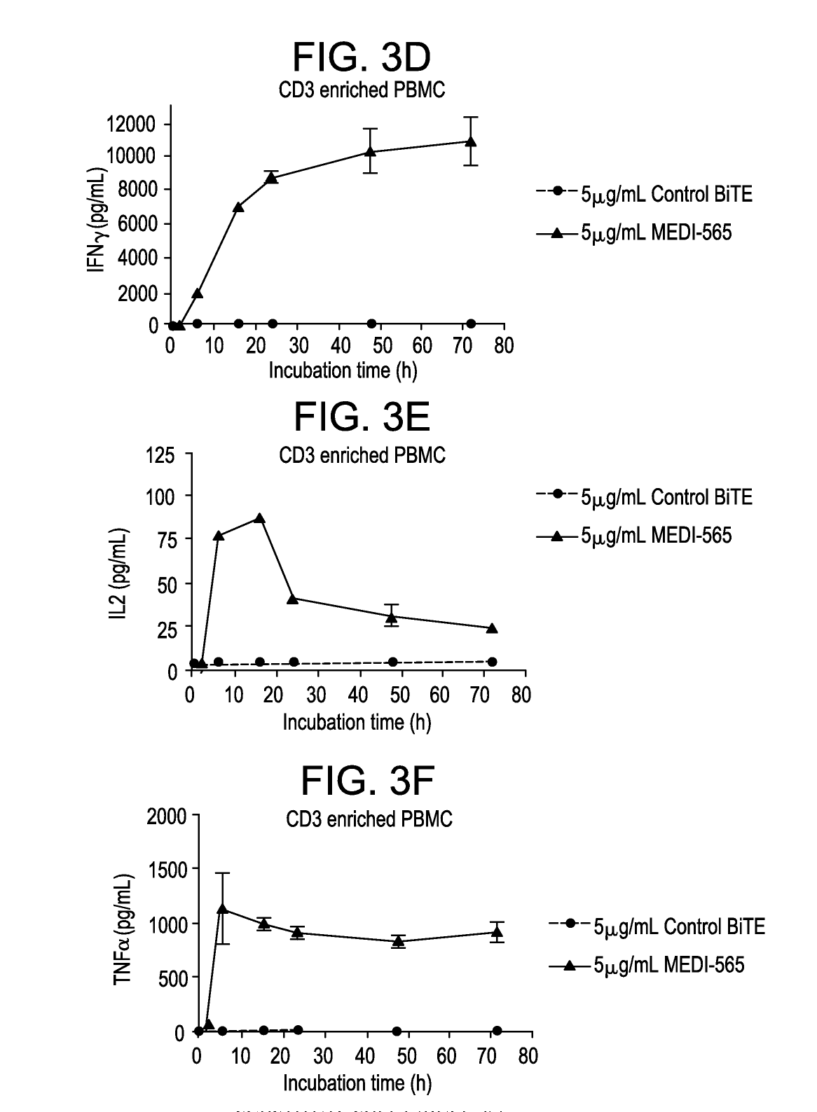

When PBMC without CD14 were used as effector cells, background levels of cytokines measured in the absence of MEDI-565 were comparable to those measured in the presence of 10 μg/mL MEDI-565 (FIG. 3). Thus, the observed cytokine secretion was likely due to alloreactivity and activation of natural killer (NK) cells, and not due to MEDI-565-mediated T cell activation. Therefore, to minimize the impact of alloreactivity, PBMC with CD3ε were used as effector cells to determine MEDI-565-mediated cytokine secretion. PBMC with CD3ε and CHO/huCEA cells were cultured at an E:T ratio of 10:1 in the presence of 5 μg/mL MEDI-565 or control BiTE® antibody for up to 72 hours.

When PBMC with CD3ε were used as effector cells, maximal cytokine levels were reached at 72 hours for IFNγ, 16 hours for IL-2, and 6 hours for TNFα. Cytokine levels in cultures of PBMC with CD3ε and CHO/huCEA in the presence of the control BiTE® antibody remained very low or below the limits of detection of the assays. Thus, PBMC enriched for CD3+ T cells had a reduced level of alloreactivity and demonstrated a time-dependent release of cytokines Based on these results, an incubation time of 24 and 72 hours could be chosen for the detection of IL-2, IL-10, TNFα, and IFNγ.

B. Determining E:T Ratios and Incubation Times for T Cell Lysis of ASPC-1 Cells, T Cell Expression of CD69 and CD25, and Release of Cytokines

Analogous to the CHO/huCEA test system, various E:T ratios were analyzed to establish the most sensitive and stable assay setup for another target cell line, the pancreatic cancer cell line ASPC-1. A fixed number of ASPC-1 cells (10000) and varying amounts of human PBMC without CD14 were co-cultured at ratios ranging from 1:2 to 20:1 with increasing concentrations of MEDI-565 for 48 hours.

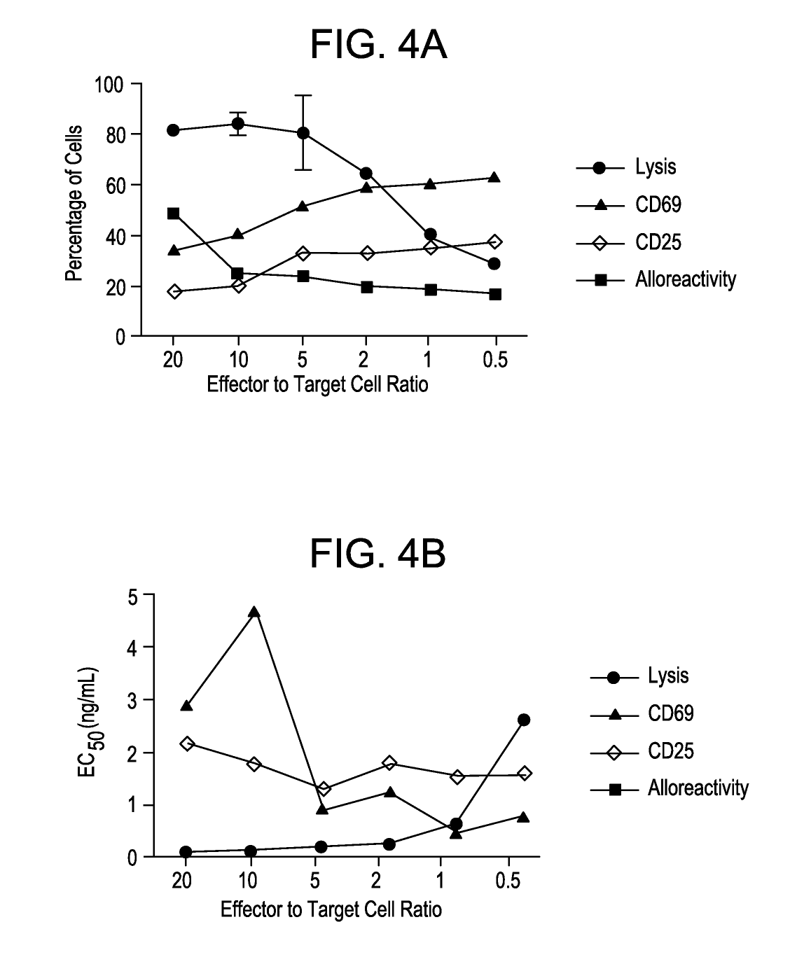

At E:T ratios of 10:1 and higher, maximal lysis was more or less constant, but declined steadily at E:T cell ratios of 5:1 and less; an E:T ratio of 20:1 also induced high levels of alloreactivity. The percentage of CD25+ and CD69+ T cells gradually decreased with increasing E:T ratios (FIG. 4). For E:T ratios below 2:1, the EC₅₀ values for target cell lysis increased considerably. However, the EC₅₀ values for CD69 expression increased noticeably with an E:T ratio above 5:1. Accordingly, the most sensitive assay system would be one with an E:T ratio between 5:1 and 2:1 (FIG. 4B).

Thus, an E:T ratio of 5:1 was chosen for the ASPC-1 test system to minimize alloreactivity (E:T<20:1), obtain maximum lysis (E:T>2:1) and a low EC₅₀ value (E:T=5:1 to 2:1).

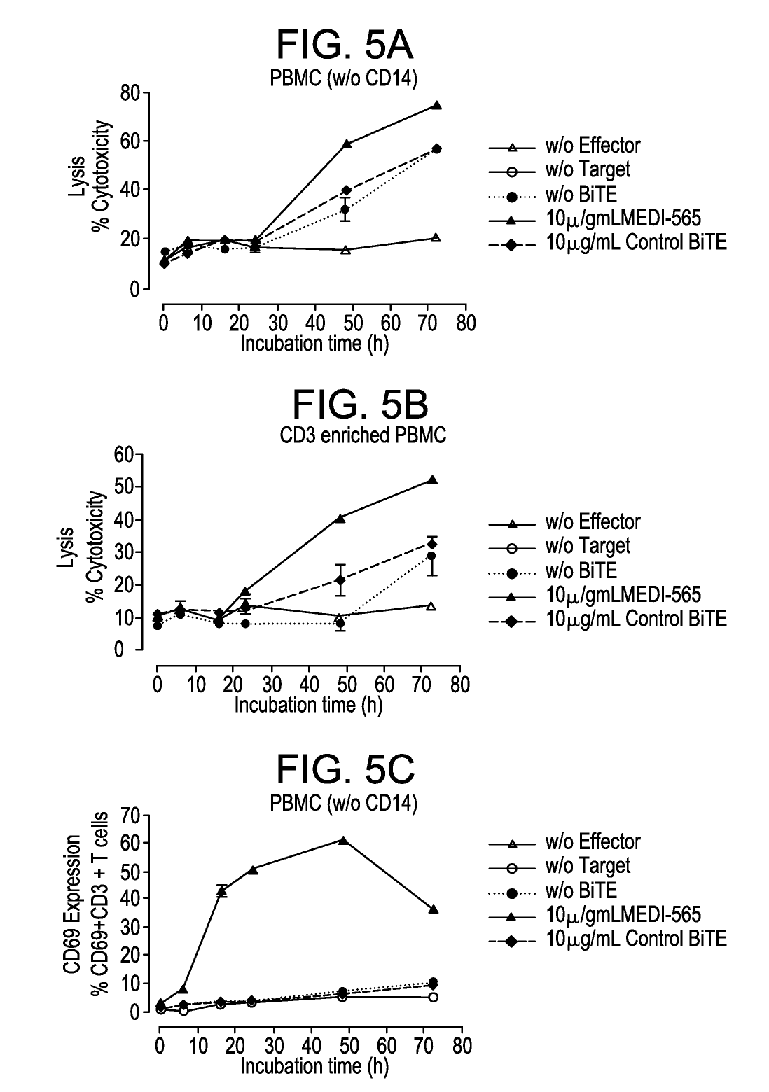

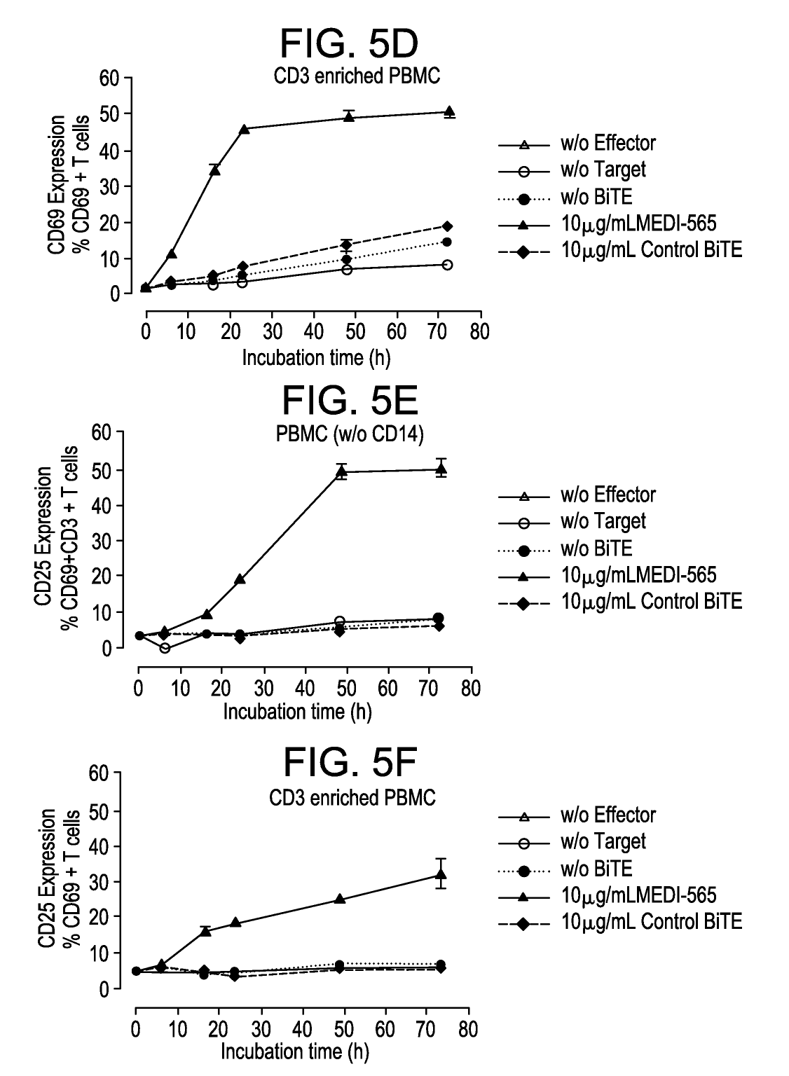

To analyze the kinetics of T cell activation mediated by MEDI-565, ASPC-1 cells were co-cultured with human PBMC without CD14 or with CD3ε at an E:T cell ratio of 5:1 in the presence of 10 μg/mL MEDI-565 or control BiTE® antibody for up to 72 hours. ASPC-1 cell lysis (FIGS. 5A and 5B) and de novo T cell expression of CD69 and CD25 (FIGS. 5C, D, E, and F) were analyzed.

ASPC-1 cell lysis increased over time (from 24 hours) and reached a maximum of about 70% for cultures containing PBMC without CD14 (FIG. 5A) and 50% for cultures containing PBMC with CD3ε (FIG. 5B), respectively, after 72-hour incubation. However, ASPC-1 cell death due to alloreactivity also increased over time and reached maximal levels after 72 hours. Thus, the highest specific lysis (about 30% for both systems) was obtained using an incubation time of 48 hours. As shown in FIGS. 5C and D, MEDI-565-induced upregulation of CD69 on T cells began at the earliest analyzed time point (6 hours). After 24 hours, roughly 50% of all T cells were activated as indicated by CD69 expression. CD25 was upregulated on about 50% of T cells using PBMC without CD14 as effector cells (FIG. 5E), but only on 30% using PBMC with CD3ε as effector cells (FIG. 5F). CD25 expression started later than upregulation of CD69 and was first measurable after 16 hours. CD25 and CD69 upregulation was also observed to a certain extent in samples devoid of BiTE® antibody, which again can probably be attributed to alloreactivity. However, compared to MEDI-565-mediated activation of T cells, the magnitude of the activation due to alloreactivity was negligible (<10%).

Thus, an incubation time of 48 hours was chosen as optimal for the MABEL determination to reduce the impact of alloreactivity and obtain an assay system with the greatest dynamic range.

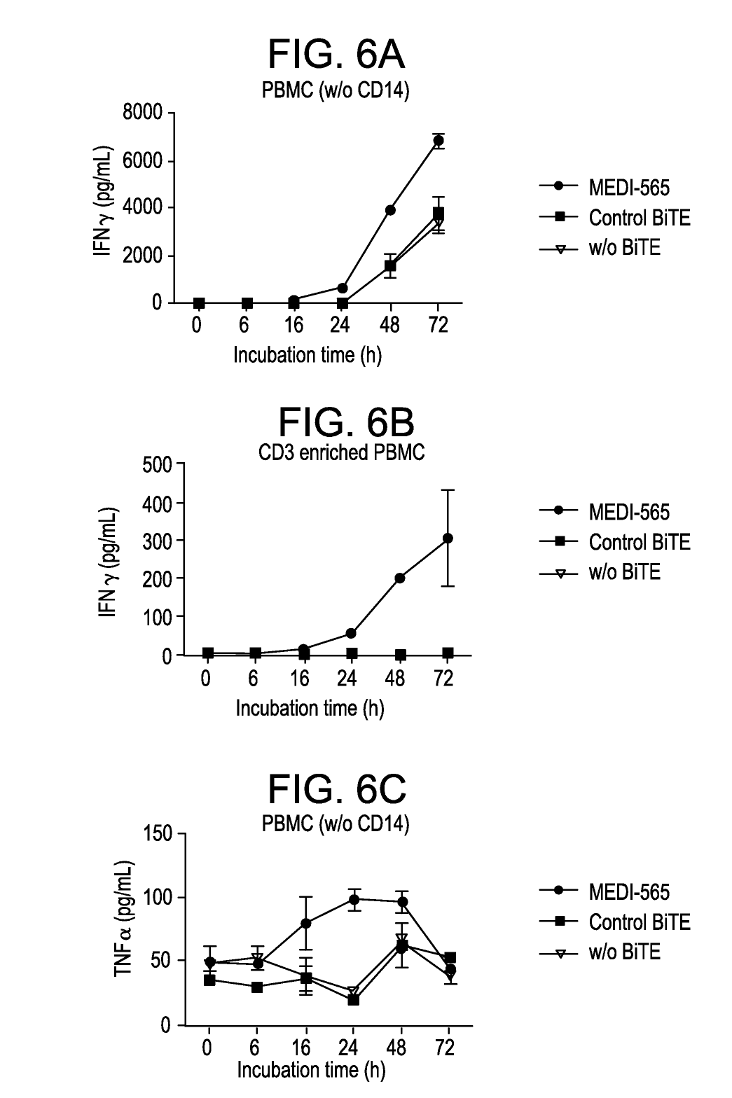

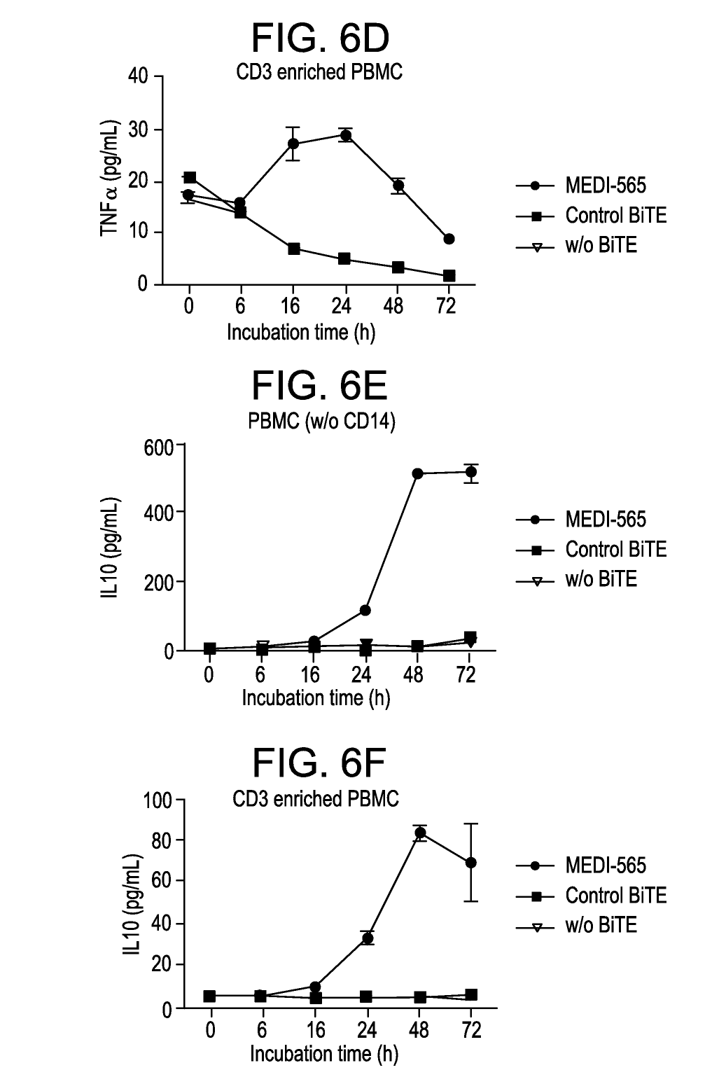

Complementing the cell-based FACS analysis, the supernatants of the above described experiments were analyzed for their cytokine content after 6, 16, 24, 48, and 72 hours of incubation time using the BD Cytometric Bead Array (CBA) Human Th1/Th2 Cytokine Kit II (BD, #551809). In general, the measured level of all analyzed cytokines (IFNγ, TNFα, IL-2, IL-6, IL-4, and IL-10) was higher using PBMC without CD14 (FIGS. 6A, C, and E) compared to PBMC with CD3ε (FIGS. 6B, D, and F) as effector cells. As observed with CHO/huCEA cells, there were high background cytokine concentrations using PBMC without CD14, most probably due to alloreactivity and activation of NK cells present in PBMC without CD14 and not in PBMC with CD3ε. Using PBMC with CD3ε as effector cells, very low levels of each cytokine were detected in the absence of MEDI-565. This correlated well with the reduced alloreactivity (compare FIGS. 5A and B). There were no MEDI-565-mediated increases in IL-4, IL-2, and IL-6 (data not shown), whereas IFNγ (FIG. 6A, B) and IL-10 (FIG. 6E, F) accumulated in the assay supernatant over time. The TNFα concentration reached a maximum after 24 hours of incubation and slowly decreased in magnitude thereafter (FIG. 6C, D).

Thus, to determine the MABEL for cytokine secretion, PBMC with CD3ε were used as effector cells to minimize the impact of alloreactivity, while an incubation time of 48 hours was chosen as optimal for the simultaneous detection of IL-10, TNFα, and IFNγ.

C. MABEL Test Assays

Effector cells isolated from different healthy donors (n≧12) were tested in the optimized FACS-based cytotoxicity assays. Co-cultures consisted of CHO/huCEA and ASPC-1 tumor cells, respectively, as target cells, and PBMC either with CD3ε or without CD14 as effector cells. The different effector cell populations were chosen first to determine the MEDI-565 MABEL with an effector cell population as close as possible to unfractionated human blood (PBMC without CD14) and second to identify the potential impact of target cell alloreactivity on the MEDI-565 MABEL.

1. MABEL Test Assays Using CHO/huCEA Cells—Cell Lysis and Activation

In total, effector cells isolated from 12 different healthy donors were tested in FACS-based cytotoxicity and T cell activation assays for each type of effector cell preparation. CHO/huCEA cells were used at an E:T ratio of 10:1 and an incubation time of 72 hours.

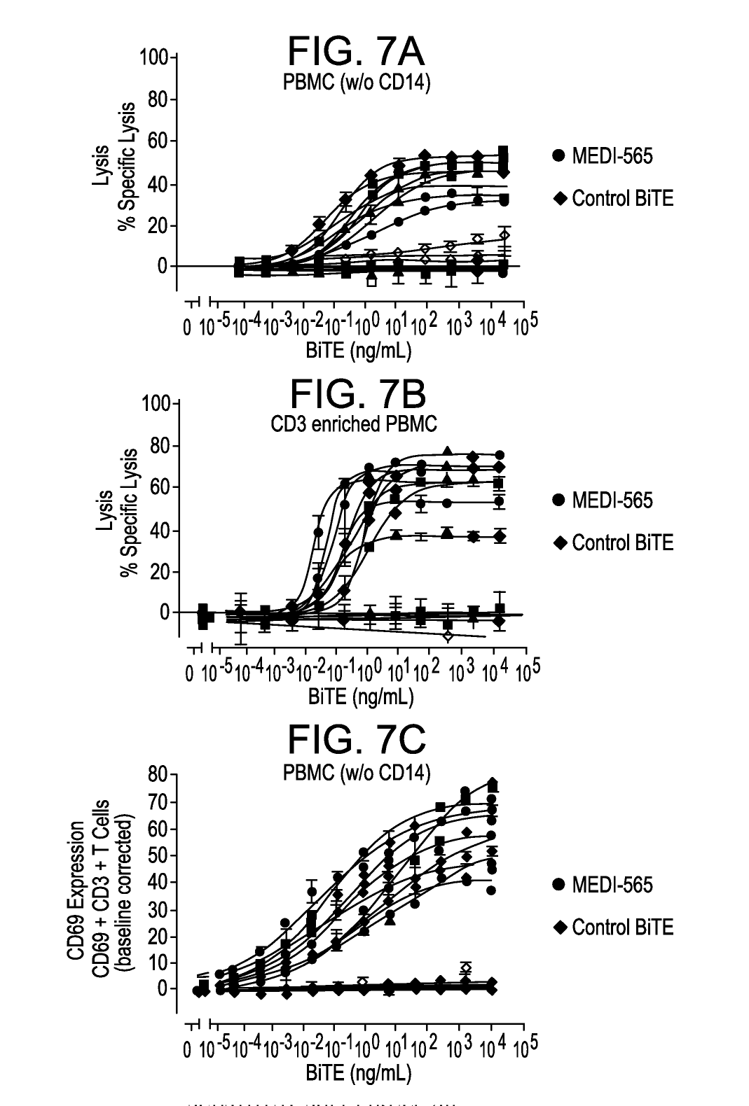

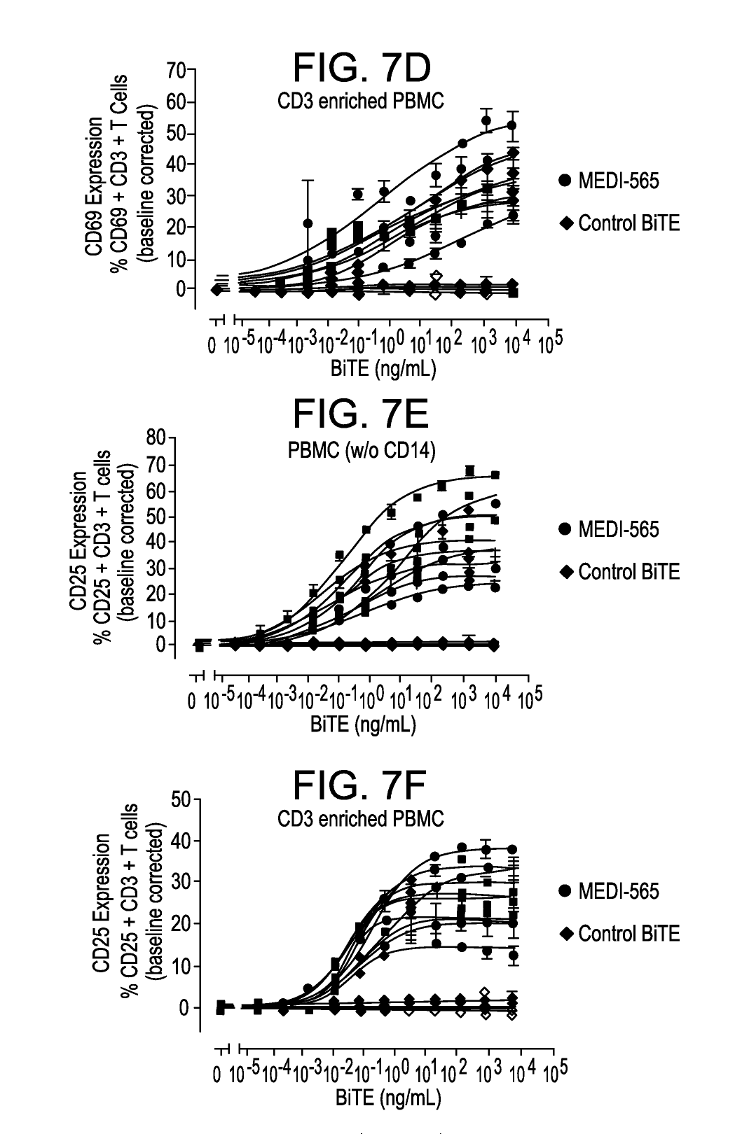

With the different PBMC preparations, MEDI-565 induced a dose-dependent specific lysis and de novo expression of CD25 and CD69 on CD3+ T cells. After 72 hours of incubation, 30% to 60% of CHO/huCEA cells were lysed using PBMC without CD14 as effector cells (FIG. 7A), whereas 40% to 80% were lysed using PBMC with CD3ε (FIG. 7B). Using PBMC without CD14 as effector cells, between 20% and 80% of all T cells became activated within 72 hours of incubation (FIGS. 7C and E). Ten percent to 60% of all T cells became activated using PBMC with CD3ε as effector cells (FIGS. 7D and F). Incubation with the control BiTE® antibody had no effect on T cell activation or target cell viability (FIG. 7).

Two of the 12 donors evaluated were used for calculation of the EC₂₀ values of specific lysis using PBMC without CD14 and with CD3ε, respectively, as effector cells. For de novo expression of CD25, 8 of 12 donors were included using either effector cell preparation. Two and one of 12 donors were valid for calculation of EC₂₀ values of CD69 de novo expression using PBMC without CD14 and with CD3ε, respectively. Most of the CD69 curves were not sigmoidal in shape, and therefore, did not qualify for data inclusion. This likely resulted from a nonuniform activation of T cell populations; MEDI-565 preferentially activates CD8+ T cells at lower concentrations than CD4+ T cells.

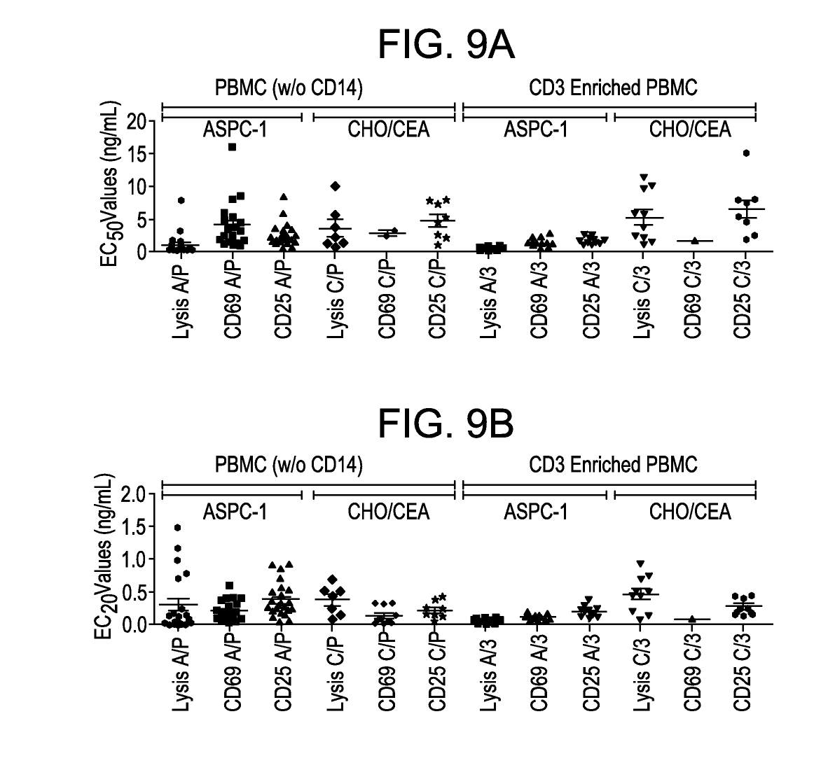

For CHO/huCEA cells, the most sensitive marker for determining MEDI-565-induced biological activity was the upregulation of the T cell activation marker CD25 (FIG. 9).

The calculated EC₂₀ values for MEDI-565-induced T cell activation as assessed by CD25 upregulation determined from the individual dose-response curves differed due to inter-donor variability. The mean EC₂₀ value for MEDI-565-induced upregulation of CD25 was calculated as 217 pg/mL with a 95% confidence interval ranging from 107 to 327 pg/mL using PBMC without CD14 and 269 pg/mL with a 95% confidence interval ranging from 162 to 376 pg/mL for PBMC with CD3ε (Table 1 and Table 2).

2. MABEL Test Assays Using ASPC-1 Cells

Effector cells from 24 different healthy donors were tested in FACS-based cytotoxicity and T cell activation assays using PBMC depleted of CD14+ (PBMC without CD14) and 12 different healthy donors were tested using enriched CD3+ cells from PBMC (PBMC with CD3ε). As target cells, ASPC-1 cells were cultured with the effector cells at an E:T ratio of 5:1 for 48 hours.

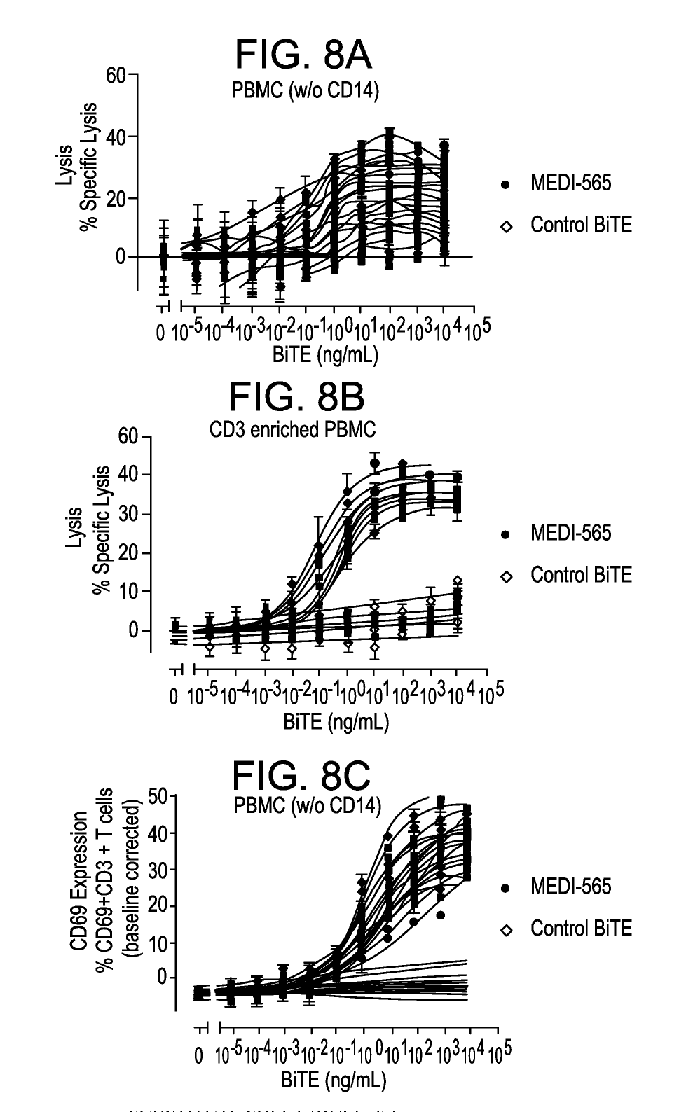

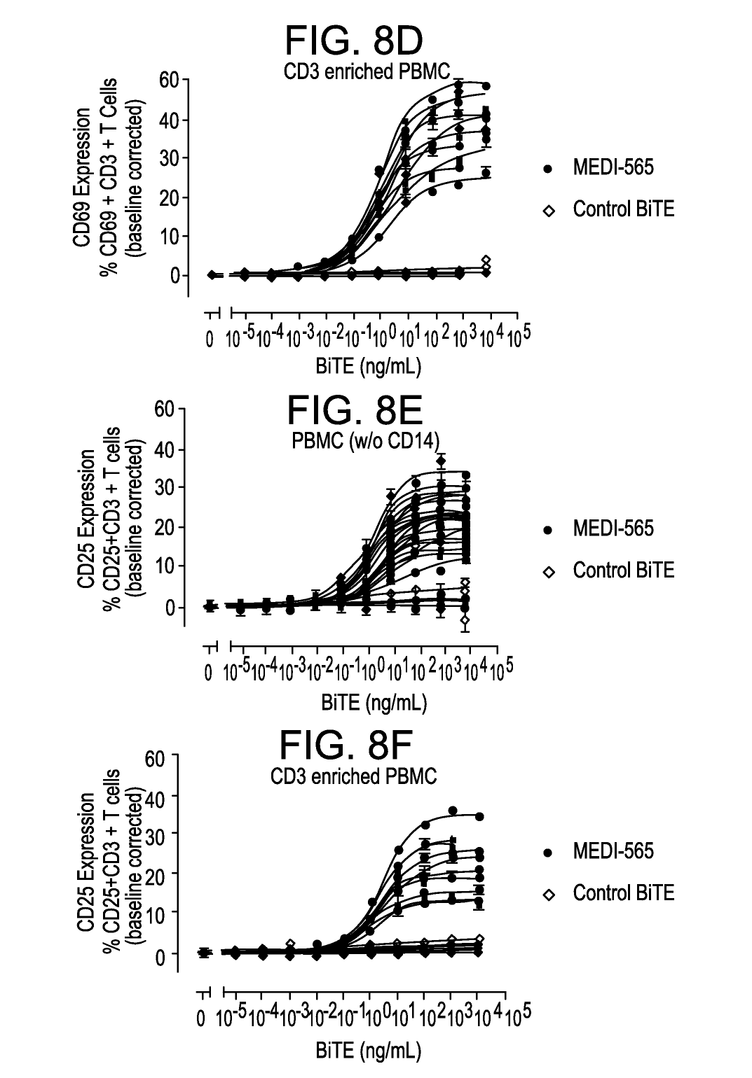

With all different PBMC preparations, MEDI-565 induced strictly dose-dependent specific lysis and de novo expression of CD25 and CD69 on CD3+ T cells. After 48 hours of incubation, 30% to 45% of ASPC-1 cells were lysed using PBMC with CD3ε (FIG. 8B) whereas less than 30% of target cells were lysed using PBMC without CD14 as effector cells (FIG. 8A). For both effector cell preparations, 10% to 50% of all T cells became activated (FIGS. 8C, D, E, and F) within 48 hours. Incubation with the control BiTE® antibody had no effect on T cell activation or target cell viability (FIG. 8).

According to the inclusion criteria (see Materials & Methods), 11 of 12 donors were included for calculation of the EC₂₀ values of specific lysis, and CD69 de novo expression using PBMC with CD3ε as effector cells. For de novo expression of CD25 on effectors PBMC with CD3ε, 10 of 12 donors were included for calculation of the EC₂₀ value. Using PBMC without CD14 as effector cells, none of the 24 specific lysis curves obtained fulfilled the criteria for data inclusion. Thus, EC₂₀ values of CD25 and CD69 de novo expression obtained with PBMC without CD14 effector cells were not considered valid, although some of the curves fit the data inclusion criteria.

For ASPC-1 cells, the most sensitive marker for determining MEDI-565-induced biological activity was specific lysis (FIG. 9). The mean EC₂₀ value for MEDI-565-induced specific lysis derived from the included 11 different donors was calculated as 50 pg/mL and the 95% confidence interval ranged from 24 to 74 pg/mL using PBMC with CD3ε (Table 1 and Table 2).

Specific lysis of ASPC-1 cells using PBMC with CD3ε as effector cells, at an E:T ratio of 5:1 and an incubation time of 48 hours, was identified as the most sensitive biomarker of MEDI-565 activity (see FIG. 9). Thus, the EC₂₀ value of specific lysis (50 pg/mL) obtained with this assay setup was determined as in vitro MABEL of MEDI-565. As noted below, the EC₂₀ values obtained and described in Example 1 did not differ in a statistically significant way from those obtained and described in Example 2 when a different preparation of MEDI-565 was used.

3. MABEL for Cytokines

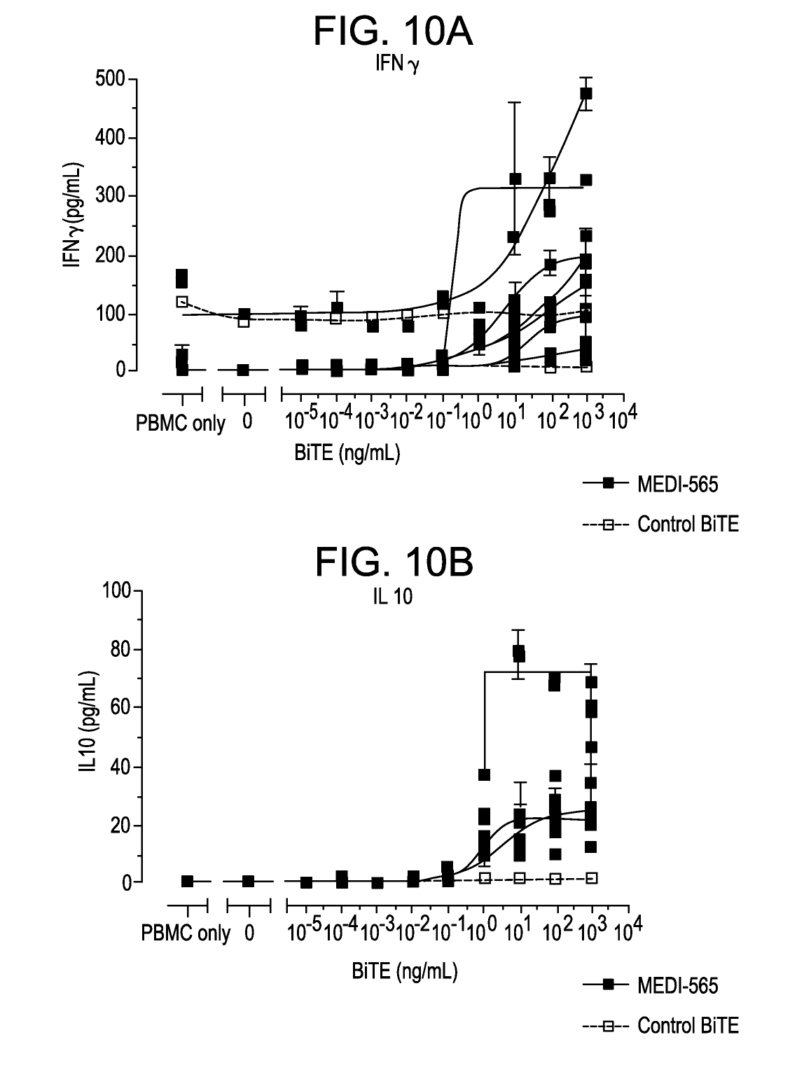

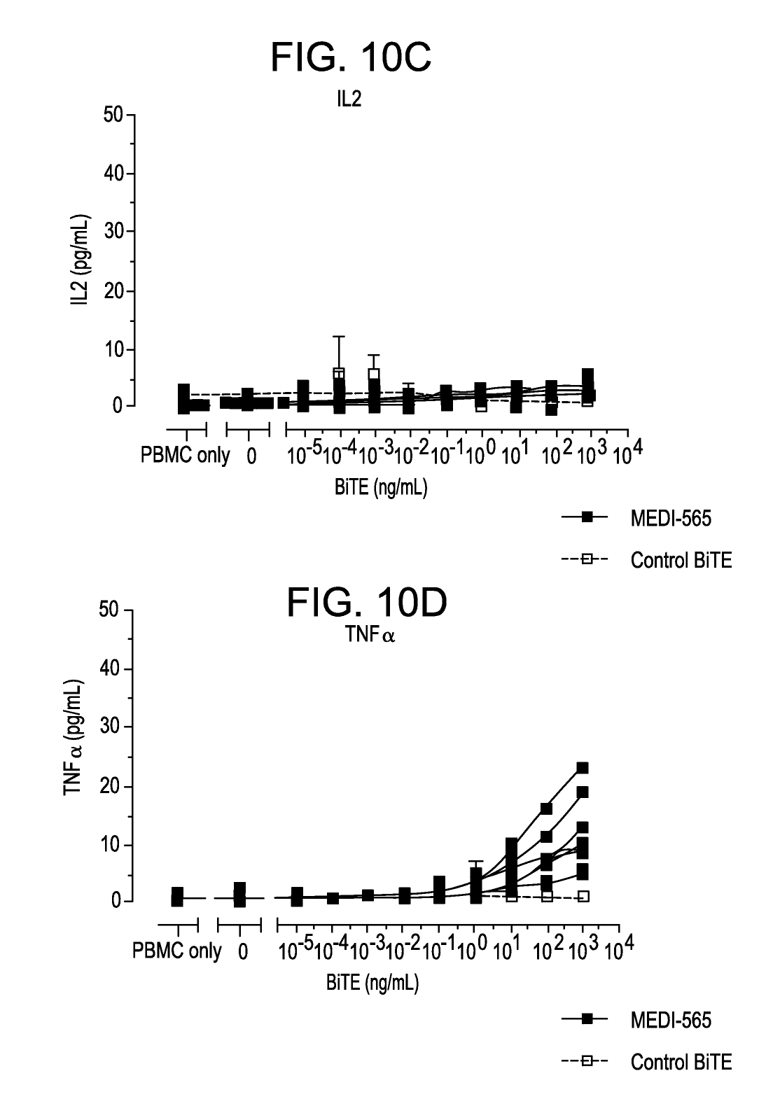

T cell activation mediated by MEDI-565 can induce cytokine secretion. Thus, MEDI-565-induced release of cytokines by CD3+ T cells in the presence of ASPC-1 tumor cells (most sensitive test system) was analyzed. We analyzed only the ASPC-1 test system more in detail as (1) it comprised the most sensitive test system and (2) analysis of cytokine secretion by CHO/huCEA cells was hampered by high background cytokine values. The assay supernatants of the 7 valid donors (see Materials & Methods) were analyzed for levels of IL-2, IL-10, TNFα, and IFNγ (FIG. 10).

In general, the cytokine response measured was highly variable for the different donors tested, and the amount of cytokines detected was quite low. No fitted dose-response curve fulfilled the criteria for data inclusion. Cytokines were only secreted at high MEDI-565 concentrations. Thus, cytokine secretion was considered a less sensitive biomarker than specific lysis and not applicable for the determination of the MEDI-565 in vitro MABEL.

4. Influence of Overnight Effector Cell Culture on Cytokine Background Level, T Cell Activation, and Target Cell Lysis

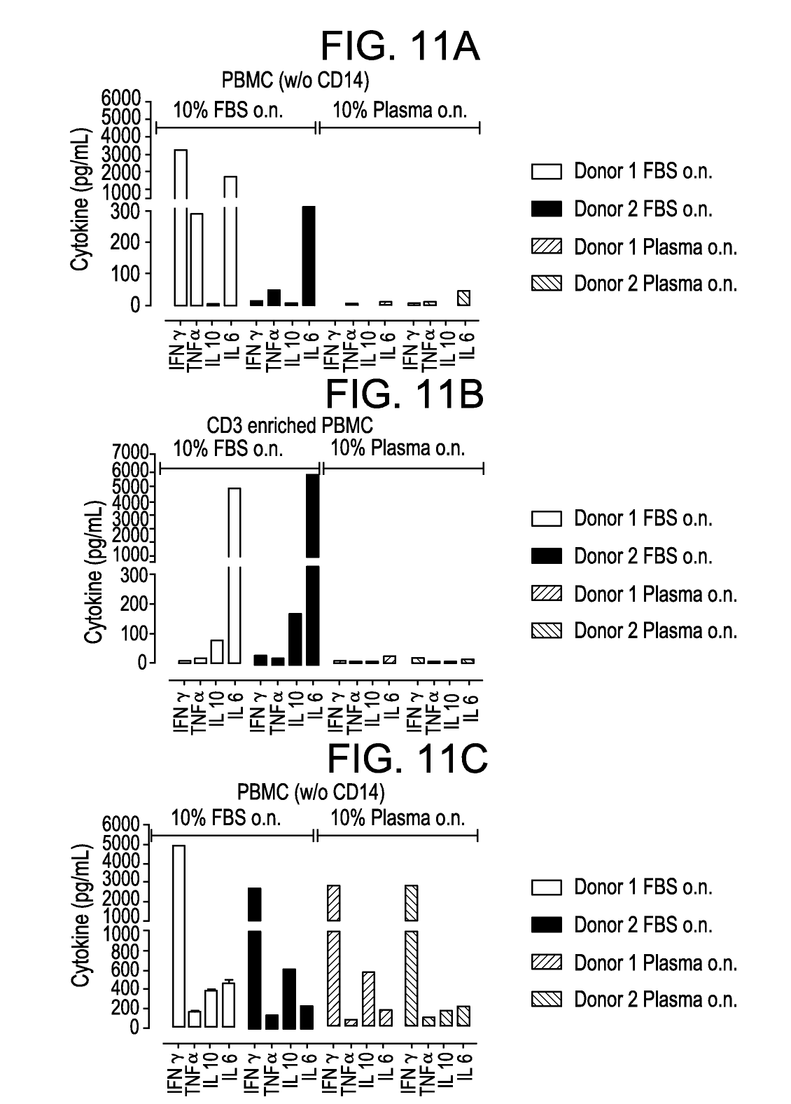

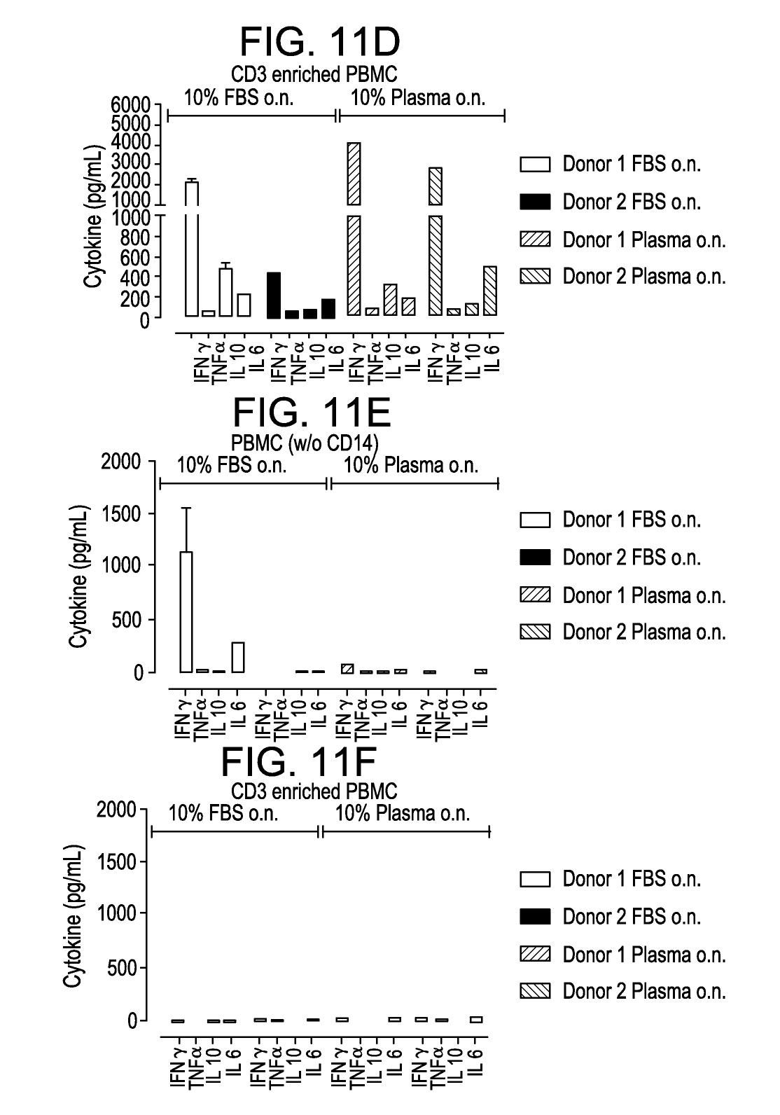

Xenogeneic serum can induce immune cell activation. Thus, the background cytokine levels after overnight co-culture was tested using as effector cells either human PBMC without CD14 (n=6, only 2 are shown) or PBMC with CD3ε (n=2), in medium containing either 10% FBS or human donor-matched plasma (FIGS. 11A and B).

No human cytokines were detected in FBS itself (data not shown). The addition of FBS to medium during overnight co-culture led to elevated levels of various cytokines in each effector cell preparation for all donors tested (FIGS. 11A and B). The set of cytokines released was different for PBMC without CD14 (IFNγ, TNFα, IL-6) and PBMC with CD3ε (IL-10, IL-6). In addition, the magnitude of release for any given cytokine differed from donor to donor (greater for PBMC without CD14). In contrast, the addition of human donor-matched plasma to medium, instead of FBS, of either overnight effector cell culture did not lead to any substantial release of the cytokines tested.

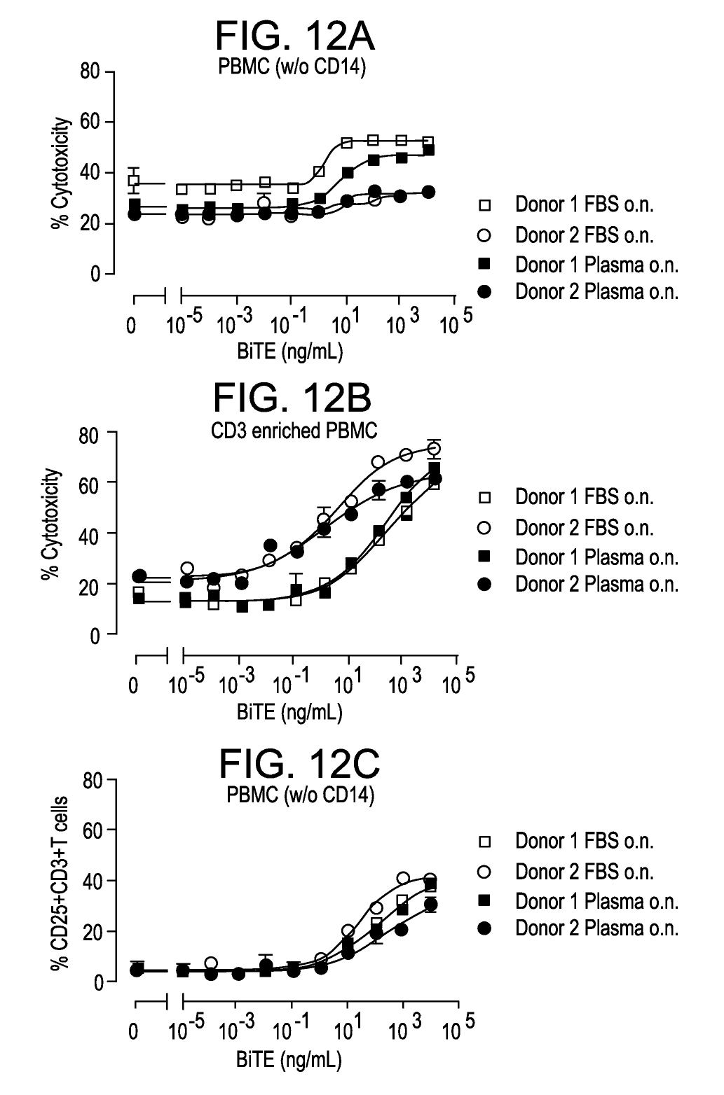

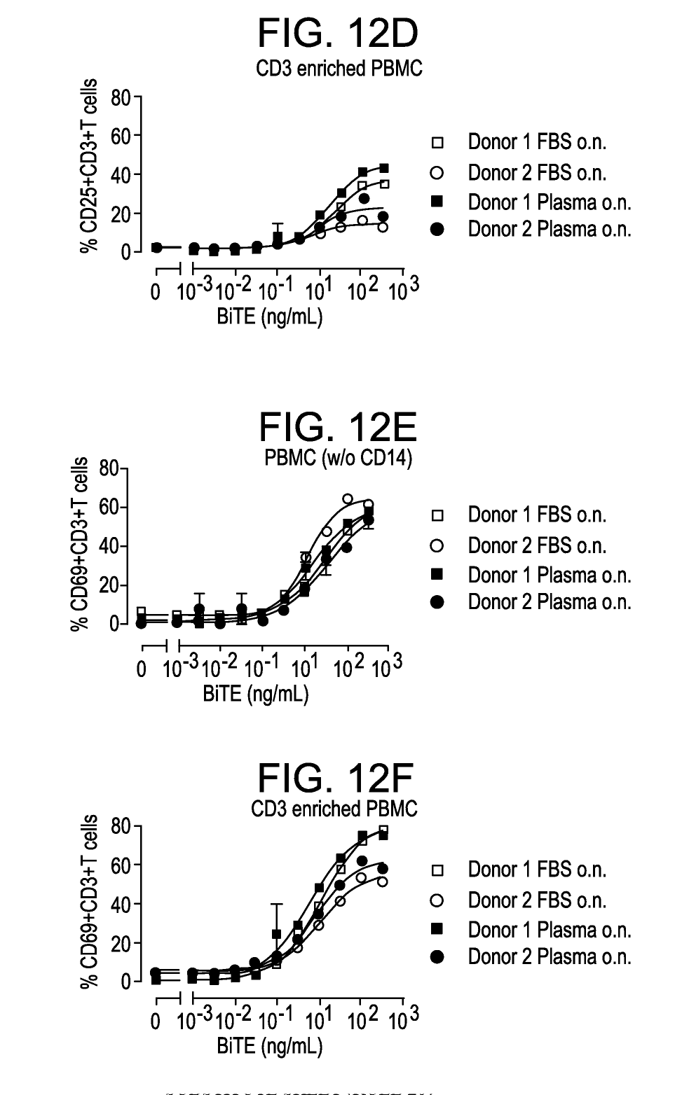

The bioactivity of each effector cell preparation was compared in the optimized FACS-based cytotoxicity assays. ASPC-1 tumor cells (E:T ratio 5:1) were incubated for 48 hours with vehicle control or the indicated concentrations of MEDI-565; supernatants were measured for levels of cytokines (FIGS. 11C, D, E, and F). The corresponding tumor cell lysis (FIGS. 12A and B), CD25 (FIGS. 12C and D), and CD69 (FIGS. 12E and F) de novo expression are shown in FIG. 12 for the various MEDI-565 concentrations tested.

For 3 of the 6 donors, the addition of FBS to medium of the overnight culture led to elevated cytokine concentrations in samples containing target cells, PBMC without CD14 effector cells and vehicle. This was completely prevented by replacing FBS by human donor-matched plasma (FIG. 11E). No elevated background cytokine levels were observed after incubating PBMC with CD3ε with target cells and treating with vehicle control regardless of whether FBS or donor-matched plasma were added to medium of the overnight culture (FIG. 11F). Cultivation with donor-matched plasma led to a decrease of total released cytokines for PBMC without CD14 during the cytotoxicity assay with MEDI-565, and to an increase in secreted cytokines for PBMC with CD3ε (FIGS. 11C and D).

However, the bioactivity of MEDI-565 was not significantly altered for the respective effector cell preparations incubated either with FBS or donor-matched plasma (FIG. 12).

Replacement of FBS by the donor-matched plasma during the cytotoxicity assay itself had no impact on MEDI-565 bioactivity or on cytokine release in any of the combinations tested (data not shown).

The results of these experiments suggested that FBS should be replaced by donor-matched plasma in overnight effector cell cultures for pivotal MABEL studies to prevent any MEDI-565 unrelated cytokine production.

### Example 2

### Determination of MABEL for MEDI-565

A. MEDI-565 Specificity

The mode of action of MEDI-565 is dependent on the simultaneous linkage of huCEA-positive tumor cells with CD3-positive T cells. To confirm this characteristic, serial dilutions of MEDI-565 were incubated in the presence of ASPC-1 tumor cells only. Additionally, mixtures of tumor and T cells were incubated in the presence of serial dilutions of the control BiTE® antibody that exclusively binds to the CD3 antigen and does not bind to CEA.

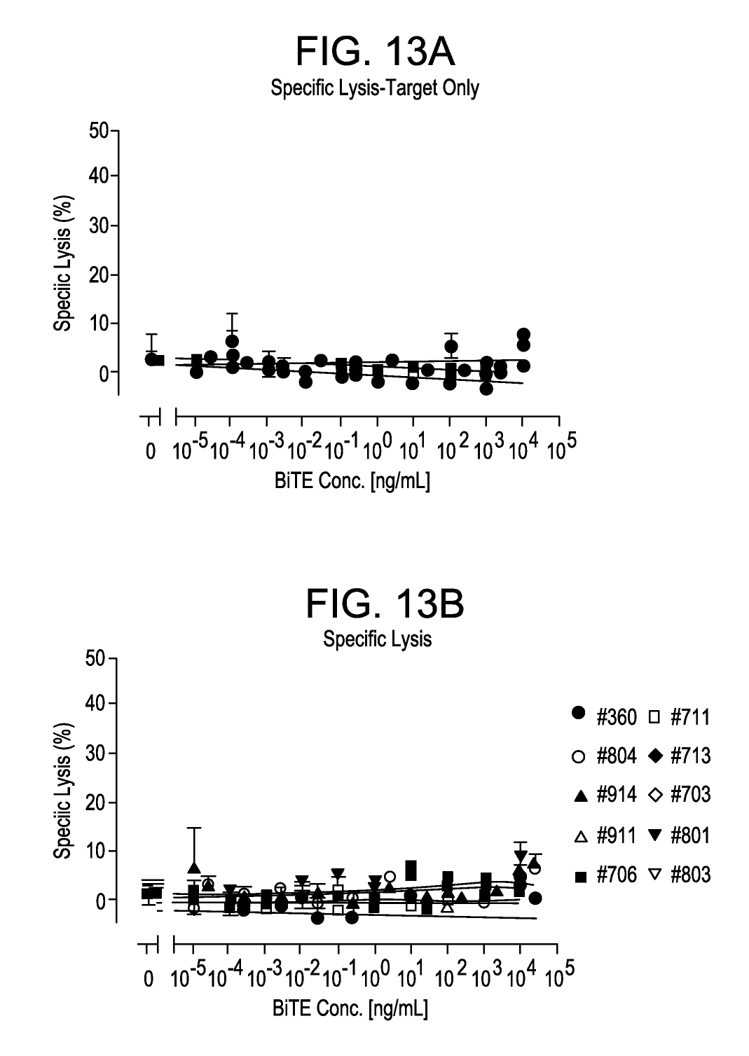

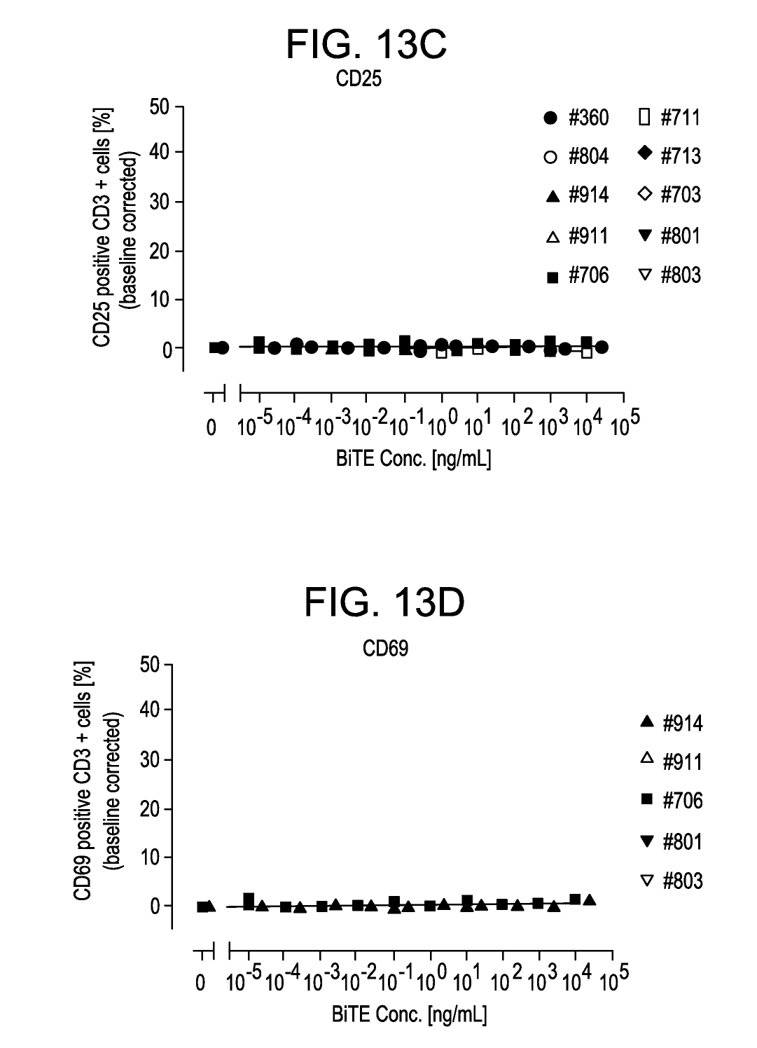

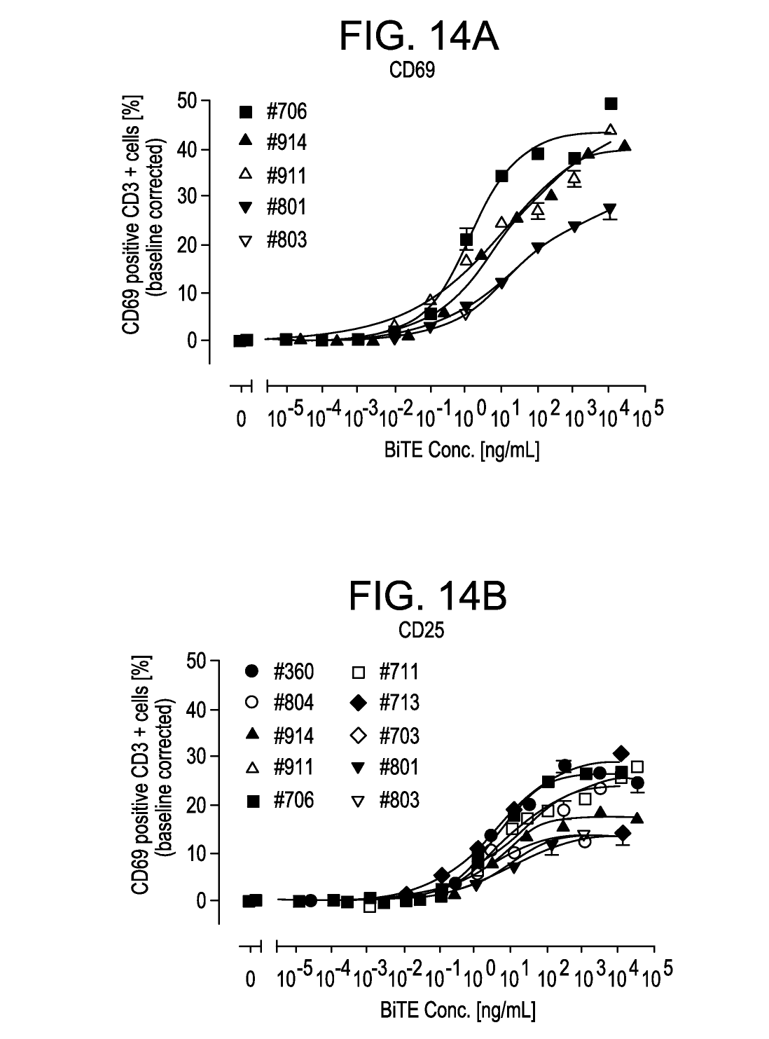

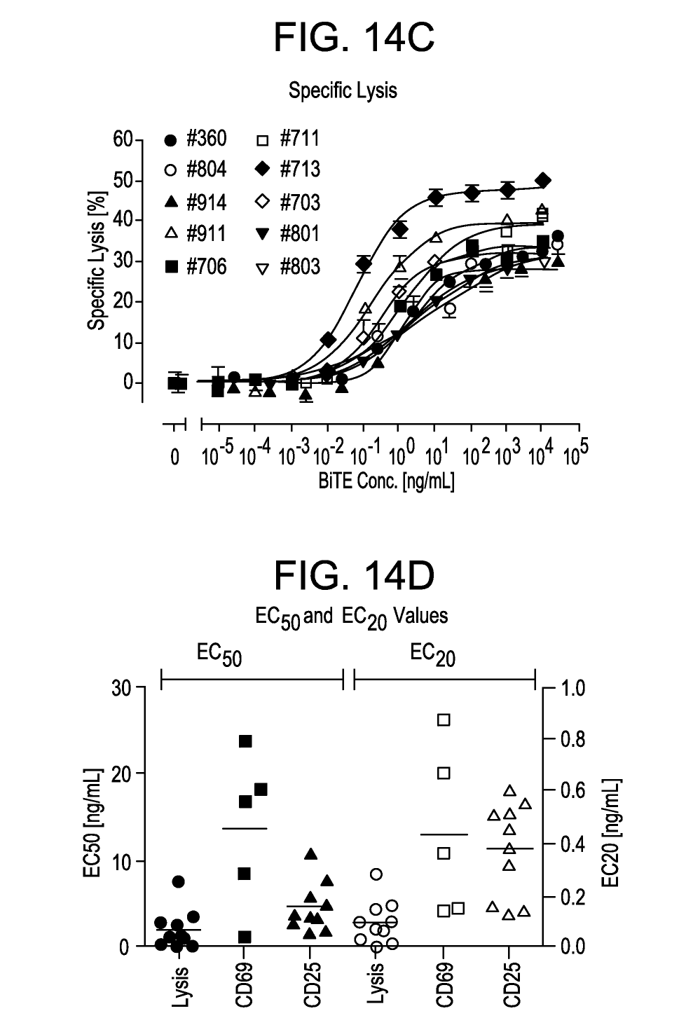

MEDI-565 had virtually no effect on tumor cell lysis in the absence of effector T cells, even up to a concentration of 25 μg/mL, demonstrating that the anti-tumor activity is entirely mediated by redirected T cells (FIG. 13A). Similarly, the control BiTE® antibody had no detectable effect on target cell lysis (FIG. 13B) or T cell activation (FIGS. 13C and D) up to 25 μg/mL in the presence of huCEA-positive tumor cells. This demonstrates that simultaneous binding of the huCEA and CD3 binding arms and concomitant linkage of tumor and T cells is required for T cell activation. In contrast, in the presence of MEDI-565, T cells, and huCEA-positive tumor cells, T-cell activation (FIGS. 14A and B) and tumor cell lysis (FIG. 14C) were observed.

B. MABEL for T Cell Activation and Tumor Cell Lysis

The lot of MEDI-565 used for the experiments described in Example 1 was a research grade lot, whereas the lot of MEDI-565 used for the experiments described in Example 2 was toxicology grade. However, the values obtained and described in Example 1 were comparable to those obtained and described in Example 2. As described below, the results from the experiments conducted in Example 2 were used to identify appropriate dosing in humans.

CD69 (FIG. 14A) and CD25 (FIG. 14B) upregulation on T cells and specific lysis of target cells (FIG. 14C) were analyzed as indicators for biological activity of MEDI-565. The MABEL was defined as the effective concentration that induced 20% of the maximal effect (EC₂₀). In total, 36 different donors were tested in FACS-based co-culture assays of ASPC-1 tumor cells and PBMC with CD3ε effector cells in the presence of serial dilutions of MEDI-565, and respective dose-response curves were generated. The curve progression was fitted by the Prism 4 software (Graph Pad Software, San Diego) and only dose response curves that fulfilled data inclusion criteria (Materials & Methods) were used to calculate the EC₂₀ values.

Tumor cell lysis experiments with 10 different donor PBMC preparations fulfilled the inclusion criteria, and thus, were used for the MABEL calculation. In addition to tumor cell lysis, MEDI-565 also induced dose-dependent upregulation of CD25 and CD69 on T cells. Inter-donor variation in the calculated EC₂₀ values for MEDI-565-induced T cell activation is explained by variability of the effector cells (FIG. 14).

Within a 48-hour incubation, between 10% and 50% of all T cells became activated at the highest concentration tested (FIGS. 14A and B) resulting in tumor cell lysis of 30% to 50% of all tumor cells at this concentration (FIG. 14C). CD25 was better suited for MABEL calculations, as CD69 curves were less often sigmoidal in shape. Accordingly, only 5 of the 10 CD69 dose-response curves fulfilled data inclusion criteria, whereas all 10 of the CD25 dose response curves (Materials & Methods) were used for EC₂₀ calculations (Table 1). The mean EC₂₀ value (±standard error of the mean [SEM]) for MEDI-565-induced upregulation of CD25 and CD69 was 441±146 pg/mL (n=10) and 378±58 pg/mL (n=5), respectively. MEDI-565-induced lysis of tumor cells was the most sensitive measure for MABEL and resulted in a mean EC₂₀ value (±SEM) of 96.9±26 pg/mL (n=10).

C. MABEL for Cytokines

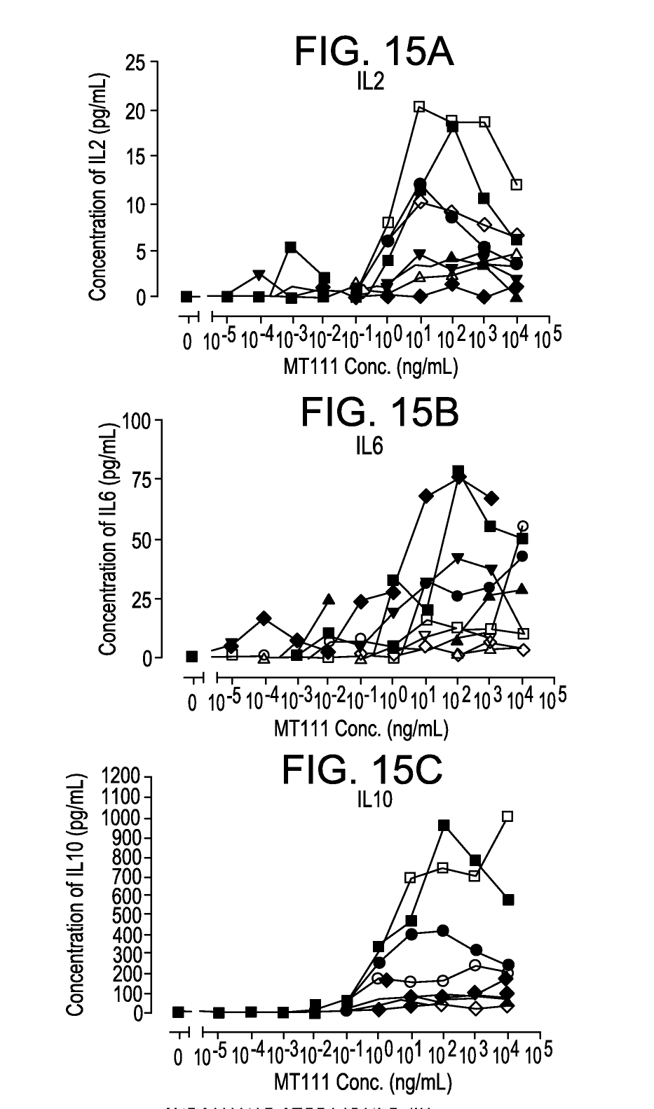

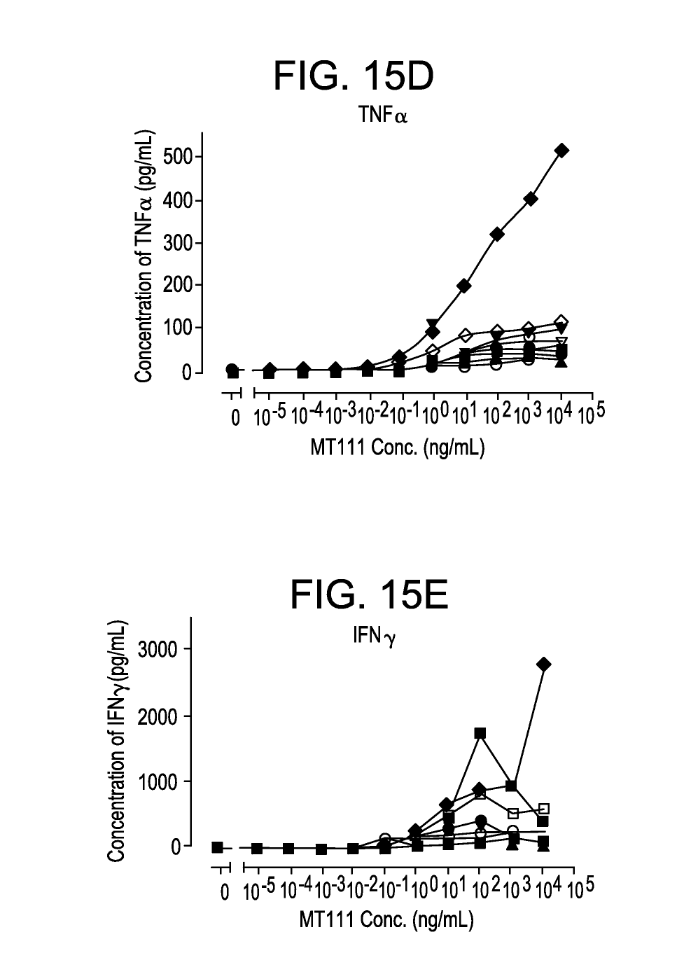

Supernatant from tumor cell lysis experiments with the 10 different donor PBMC preparations that fulfilled the inclusion criteria were analyzed for IL-2, IL-6, IL-10, TNFα and IFNγ (FIG. 15). Few valid sigmoidal dose-response curves were obtained for the analyzed cytokines independent of the used donor. For IL-2, only one valid curve was obtained, no valid curve was obtained for IL-6, 2 valid curves were obtained for IL-10, 3 curves were valid for TNFα, and no curve was valid for IFNγ. The amount of cytokines detected was generally quite low and cytokines were secreted only at high MEDI-565 concentrations (above the relevant EC₂₀). Accordingly, MEDI-565-induced cytokine release was a less sensitive measure for MABEL (EC₂₀>MABEL pg/mL) compared to other activities measured (e.g., cell lysis). Results are summarized in Table 4.

D. Calculation of Fractional Receptor Occupancy

At equilibrium binding conditions, the fraction (F) of all receptor molecules that are bound to an antibody can be calculated if the concentration and the dissociation constant (K_{D}) of the respective antibody are known, according to equation 5 (Materials & Methods):

F=[mAb]/([mAb]+K_{D})

Fractional receptor occupancy of 20% is acceptable for first-in-man clinical studies. According to the above formula, at tolerated fractional receptor occupancy of 20%, the serum concentration for MEDI-565 can be calculated as follows:

[mAb]=F*K_{D}/100−F

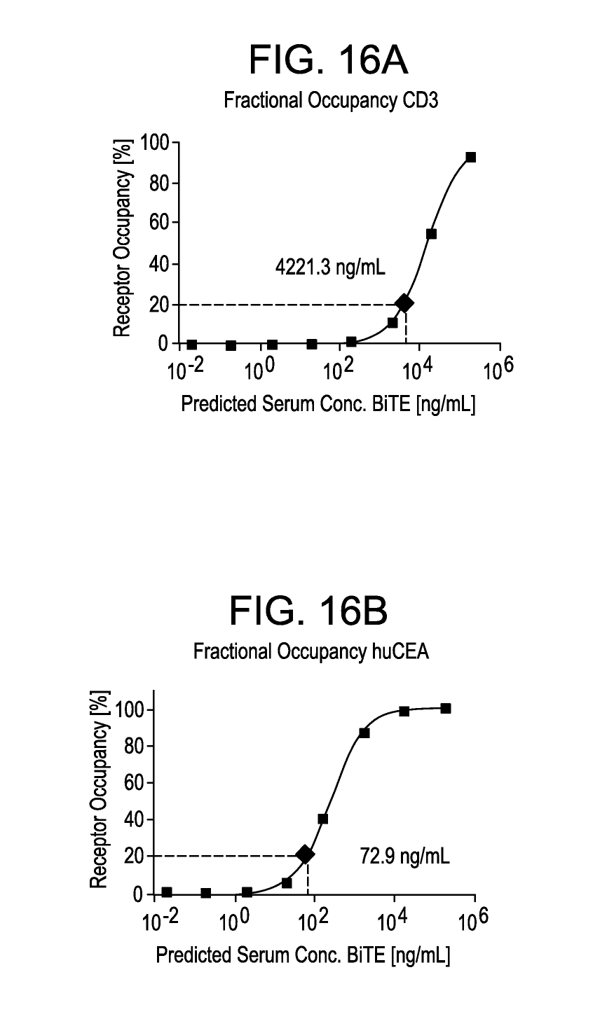

Together with the K_{D} values of the CD3 (K_{D}=307 nM) and the huCEA binding arms (K_{D}=5.3) fractional receptor occupancy versus serum concentration curves for MEDI-565 have been calculated for the CD3 and huCEA target (FIG. 16).

FIG. 16 shows the predicted serum concentrations for CD3 (FIG. 16A) and huCEA (FIG. 16B) at which a fractional receptor occupancy of 20% will be reached. In the case of MEDI-565, 20% receptor occupancy for CD3 will be reached at a free antibody concentration of 4221.3 ng/mL and for huCEA at a free antibody concentration of 72.9 ng/mL.

The calculated serum concentrations, at which fractional receptor occupancy of 20% were achieved, were above the EC₂₀ value of specific lysis calculated for ASPC-1 cells (Table 3).

### Example 3

### Non-Clinical Pharmacology, Pharmacokinetics, and Toxicology Studies

MEDI-565 specifically and selectively binds to a nonlinear, conformational epitope in human CEA with a high binding affinity; it cross-reacts with chimpanzee and cynomolgus monkey CEA. In addition, MEDI-565 specifically binds to human CD3 with a low binding affinity, and cross-reacts with chimpanzee CD3, but not with cynomolgus monkey or mouse CD3. Concomitant binding of MEDI-565 to CEA and CD3 over a wide range of E:T ratios led to the activation of primarily CD3+ T cells and the subsequent killing of cells expressing CEA. In vitro cytotoxicity assays revealed that activation of T cells by MEDI-565 was specific and selective. At the same time, T cells expanded, increased cell surface expression of activation markers, and released proinflammatory cytokines, perforin, and granzyme B. Importantly, MEDI-565 did not activate T cells in the presence of cells lacking expression of CEA.

MEDI-565 was tested in preclinical models of cancer employing human tumor cell lines mixed with human T cells and grown in mice. Treatment with MEDI-565 inhibited the growth of CEA-expressing cancer cells in cancer models of colonic, pancreatic, lung, and stomach origins. Moreover, the growth of colonic cancer cells expressing CEA was inhibited after IV and SC administration of MEDI-565. MEDI-565 did not inhibit the growth of cancer cells in the absence of human T cells or in the absence of CEA expression on cancer cells. These results demonstrated that the expression of CEA on cancer cells and the presence of CD3+ T cells are essential for the activity of MEDI-565.

A pharmacokinetics (PK) and bioavailability study was conducted in cynomolgus monkeys to establish exposure parameters for MEDI-565 following a single IV or SC administration. The human PK profile for MEDI-565 was predicted based on its PK parameters in cynomolgus monkeys and adjusted according to the principles of allometric scaling. Data from these in vitro and in vivo studies were collectively used to estimate the MABEL of MEDI-565, and to determine a dosing regimen for human administration. In addition, two tissue cross-reactivity studies of MEDI-565 were performed, one against a cynomolgus monkey tissue panel and one against a tissue microarray containing a panel of normal human tissues. Finally, a tissue cross-reactivity study against a full panel of cryopreserved normal adult human tissues was performed to complete the nonclinical safety assessment of MEDI-565.

First, to assess the effects of MEDI-565 on the in vivo growth of cancers that express human CEA, antitumor studies were performed in mice. Immunocompromised SCID mice were inoculated with combinations of human T cells and various human cancer cell lines. This model was utilized because MEDI-565 does not cross-react with mouse CD3 and mice do not endogenously express CEA. Despite the relatively short serum half-life of MEDI-565 in mice (2.5 to 5 hours), daily IV or SC administrations of MEDI-565 (range of 1 to 20 μg/mouse) for 5 days resulted in sufficient levels of exposure to inhibit the growth of cancers expressing CEA in a dose-dependent manner. Inhibition of growth was observed in cancers of colonic (LS174T, maximal tumor growth inhibition [TGI] of 99%), pancreatic (HPAC, maximal TGI of 72%; HPAF II, maximal TGI of 78%), lung (H727, maximal TGI of 53%), and stomach (MKN45, maximal TGI of 52%) origins, and was dependent on the presence of human T cells and the expression of CEA on cancer cells. These studies demonstrated that MEDI-565 has potent and selective in vivo anti-cancer activity.

Other than the chimpanzee, no pharmacologically relevant animal species exist for toxicology testing of MEDI-565. Two hybrid surrogate BiTE® antibody molecules, cyS111 and hyS111, were generated to develop a pharmacologically relevant animal species model for predicting the human toxicity of MEDI-565. The in vitro and in vivo pharmacodynamic activity of hyS111 and the in vitro pharmacodynamic activity of cyS111 were compared to that of MEDI-565; nonspecific activity and different functional characteristics than those of MEDI-565 were observed. These findings suggested that hyS111 and cyS111 would not represent the specific activity and effects of MEDI-565, and thereby limit their utility in nonclinical toxicity studies. Thus, no in vivo pharmacology studies were conducted with MEDI-565 in a pharmacologically relevant species.

A. Tissue Cross-Reactivity Studies

1. A Cynomolgus Monkey Tissue Cross-Reactivity Study

A tissue cross-reactivity study was conducted with MEDI-565 on cryosections of normal tissues from cynomolgus monkeys. The tissue panels used as the test system included cerebrum, colon, lymph node, skin, small intestine, stomach, and thymus. Experimental conditions were established in an immunohistochemistry study, where MEDI-565 was applied in titration runs to the positive (LS1034; human carcinoma cells expressing CEA) and negative (HCT-15; human colorectal adenocarcinoma cell line lacking CEA) control cell lines. In this study, the optimal concentration to detect CEA on LS1034 control cells was 0.5 μg/mL. Thus, the study on normal cynomolgus monkey tissues was conducted with MEDI-565 at concentrations of 0.5, 5, and 50 μg/mL. The highest concentration was selected to cover the possibility of any low affinity binding to tissues. The normal cynomolgus monkey tissues that had staining with MEDI-565 included colonic epithelium at all concentrations of MEDI-565 and gastric epithelium only at the highest concentration of MEDI-565. Staining was limited to the cell membranes and was consistent with that already known in human tissues (Hammarstrom, 1999).

2. Human Tissue Cross-Reactivity Study 1

A tissue cross-reactivity study with MEDI-565 on cryosections of normal tissues from healthy humans was conducted. The human tissue panel used as the test system was included in a 52-core tissue microarray (TMA) representative of 26 normal organs from two donors.

Experimental conditions were established in immunohistochemistry (1HC) experiments, where MEDI-565 was applied at concentrations of 0.1, 0.5, 1, 2.5, 10, and 25 μg/mL to the positive (LS1034, human carcinoma cell line expressing CEA) and negative (HCT-15; human colorectal adenocarcinoma cell line lacking CEA) control cell lines, as well as brain and lymph node. The optimal concentration to detect CEA on LS1034 control cells was 0.5 μg/mL. The optimal concentration of MEDI-565 to detect T cells within lymph node was 10 μg/mL. Based on these results, the final study in the normal human TMA was performed at concentrations of 0.5 and 50 μg/mL of MEDI-565. The higher concentration was selected to cover the possibility of any low affinity binding to tissues.

Human tissue staining observed with MEDI-565 included expected staining (membrane) of lymphocytes (T cells) within multiple tissues (breast, small intestine, skin, lymph node, spleen, thymus, and tonsil). Mucous neck cells of the stomach (one donor) were stained with MEDI-565, but not with the assay control.

3. Human Tissue Cross-Reactivity Study 2

MEDI-565 was applied to cryosections of normal human tissues at concentrations of 1 and 25 μg/mL. MEDI-565 had moderate to strong reactivity at both concentrations with the positive-control cells LS 1034 (human carcinoma cell line expressing CEA); it did not specifically react with the negative-control cells HCT-15 (human colorectal adenocarcinoma cell line lacking CEA). The concentrations of MEDI-565 were selected in preliminary staining runs against the positive- and negative-control cells, and 1 μg/mL was determined to be the optimal concentration. A 25-fold increase in concentration was included in this study to cover the possibility of any low affinity binding to tissues.

The control BiTE® antibody, a bispecific single-chain antibody derivative directed against an irrelevant protein and human CD3, did not bind to the positive- or negative-control cells and staining was not observed when the primary antibody was eliminated from the staining reaction (secondary antibody alone; assay control). The results were consistently reproducible. Binding of MEDI-565 to CD3 was not evaluated in this study; in preliminary staining runs, MEDI-565 was demonstrated to be a poor biological reagent for the immunohistochemical detection of CD3 in tissue sections. The reason for this observation is unknown. Characterization of MEDI-565 demonstrated no reductions in binding affinity to CD3 and no changes in potency in cell-based activity assays. The moderate to strong reaction of MEDI-565 with the positive-control cells and the lack of specific reactivity with the negative-control cells, as well as the lack of binding by control BiTE® antibody and secondary antibody alone to the control cells, indicated this immunohistochemistry assay was specific and reproducible.

Staining specific for MEDI-565 was present on the cell membrane of superficial epithelial cells in the mucosal layer of the esophagus, tonsil, cervix, and colon. Staining was also observed on the cell membrane of epithelial cells of Hassall's corpuscles in the thymus and the superficial epithelium in the cornea. All other tissues did not stain with MEDI-565. The epithelial staining observed was consistent with the known expression of CEA as cited in the literature (Hammarström, 1999; Suzuki et al., 2009; Tendler et al., 2000).

D. General Toxic Signs and Pathologic Effects

Formal in vivo toxicity studies of MEDI-565 were not performed given the lack of a suitable pharmacologically relevant animal model to assess toxicity. Results from in vitro studies on human cells, using the most sensitive test systems and assay conditions, identified the MABEL of MEDI-565 to be 0.097 ng/mL. Additional in vitro studies demonstrated that the ability of MEDI-565 to induce cytokine release and proliferation of T cells (in particular CD3+ T cells) required engagement of both CD3 on T cells and CEA on target cells; this suggested that in vivo activation of T cells in the absence of the CEA target is not likely. Results from a PK and bioavailability study in male cynomolgus monkeys demonstrated only treatment-related, reversible erythema and bruising at the administration site following IV injection. There were no changes in clinical observations, body weight, serum chemistry, hematology, coagulation, or urinalysis parameters. Results from tissue cross-reactivity study on a full panel of human tissues showed expected staining of epithelial cells consistent with literature reports describing the expression of CEA.

Single-dose toxicity studies and repeat-dose toxicity studies of MEDI-565 were not conducted due to the lack of a suitable pharmacologically relevant animal model to assess toxicity. Nevertheless, various in-life toxicity endpoints were assessed as components of a nonterminal PK and bioavailability study performed in male cynomolgus monkeys after IV and SC administrations of 0.5 mg/kg MEDI-565. The study design is shown in Table 5.

In-life observations included clinical signs (morbidity and/or mortality [twice daily]; cage-side observations and food consumption [once daily]), body weight (Weeks −2, −1, and weekly thereafter starting on Day 7), inspection of injection site (predose, 2 to 4 hours post dose, daily for 4 days or until resolution following each dose), and clinical pathology parameters (including serum chemistry, hematology, coagulation, and urinalysis [prestudy and Day 14]). Blood samples were collected for PK analysis from 5 minutes to 96 hours following IV and from 5 minutes to 120 hours following SC administrations. After the last sample was collected, the animals were returned to the testing facility animal colony.

MEDI-565, administered either IV followed by SC (Group 1), or SC followed by IV (Group 2), was generally well tolerated. There were no MEDI-565-related changes in clinical observations, body weight, serum chemistry, hematologic, coagulation, or urinalysis parameters. Treatment-related erythema and bruising were noted at the site of administration following IV injection in all animals, which resolved by the end of the study. These findings were considered likely to be procedure related.

E. Pharmacokinetics in Animals

The PK of MEDI-565 was primarily assessed in dose-range-finding studies in mice and in a study in cynomolgus monkeys. MEDI-565 does not bind to CD3 in mice or cynomolgus monkeys; and rodents do not endogenously express CEA. Following single dose IV administration in mice and cynomolgus monkeys, serum concentrations of MEDI-565 declined with a rapid initial distribution/elimination phase followed by a slower terminal elimination phase.

In CD-1 mice (using a PK assay that measures only the CD3 arm of MEDI-565 [CD3 PK assay]), the systemic clearance (CL) of MEDI-565 was 76 to 94 mL/h/kg; the apparent volume of distribution at steady state (Vₛₛ) was 117 to 182 mL/kg; the t_{1/2z} was 5 to 5.3 hours; the SC tₘₐₓ was 1 hour; and the SC bioavailability was 27% to 36%. Although the terminal phase half-life was 5 hours, the AUC₀₋₄ₕ was approximately 90% of the AUC_{inf}, representing a significant decrease in serum concentrations of MEDI-565 by 4 hours postdose, and indicating that elimination of MEDI-565 may be better reflected by the initial phase half-life of 0.2 hour.

In C57BL/6 mice (using a PK assay that measures both the CD3 and CEA arms of MEDI-565 [whole molecule assay]), the CL of MEDI-565 was 320 mL/h/kg in wild-type mice and 336 mL/h/kg in mice transgenic for human CEA; the Vₛₛ was 84 mL/kg in wild-type mice and 114 mL/kg in mice transgenic for human CEA; and the t_{1/2z} was 2.5 hours in wild-type mice and 3.5 hours in mice transgenic for human CEA. The AUC₀₋₁ₕ was approximately 90% of the AUC_{inf}, representing a significant decrease in serum concentrations of MEDI-565 by 1 hour postdose. The t_{1/2α} was 0.12 hour in wild type mice and 0.13 hour in mice transgenic for human CEA.

In cynomolgus monkeys (using the CD3 PK assay), the CL of MEDI-565 was 67 to 87 mL/h/kg; the Vₛₛ was 220 to 284 mL/kg; the t_{1/2z} was 17 to 19 hours; the SC tₘₐₓ was 6 to 7.3 hours; and the SC bioavailability was 73% to 82%. The AUC₀₋₄ₕ was about 90% of the AUC_{inf}, representing a significant decrease in serum concentrations of MEDI-565 by 4 hours postdose. The t_{1/2α} was 0.37 hour.

Intravenous administration of MEDI-565 resulted in a Vₛₛ of 117 to 182 mL/kg in CD-1 mice and 220 to 284 mL/kg in cynomolgus monkeys, indicating distribution into extracellular spaces.

The PK of MEDI-565 was linear over the dose range studied in CD-1 mice (0.75 to 3 mg/kg). In cynomolgus monkeys, the PK of MEDI-565 was only studied at dose a level of 0.5 mg/kg.

MEDI-565 is most likely degraded via normal protein catabolism, which is not dependent on cytochrome P450 (CYP) enzymes. Due to its size (approximately 54 kDa), MEDI-565 is likely to be renally excreted. The end products of catabolism of MEDI-565 (amino acids) are expected to be incorporated into the endogenous amino acid pool with a portion of it being excreted. There are no known reactive metabolites of MEDI-565.

F. Projection of Human Dose

Formal in vivo toxicity studies of MEDI-565 were not performed given the lack of a suitable pharmacologically relevant animal model to assess its toxicity. Results from in vitro studies using human tissue culture test systems identified the MABEL of MEDI-565 to be 0.097 ng/mL based on the lowest EC₂₀ value determined using the most sensitive measure for determining bioactivity of MEDI-565 (T-cell-mediated lysis of cancer cells). Additional in vitro studies demonstrated that the ability of MEDI-565 to induce cytokine release and proliferation of T cells required engagement of both CD3 on T cells and CEA on target cells; therefore, in vivo activation of T cells in the absence of CEA expression is not anticipated.

Human PK parameters for MEDI-565 were predicted based on PK parameters determined in cynomolgus monkeys, and adjusted according to the principles of allometric scaling. A 3-compartment model was fit to serum MEDI-565 concentration versus time data in cynomolgus monkeys to estimate the PK parameters. The mean actual body weight (BW) of cynomolgus monkeys (3.55 kg) was used in the scaling. The BW of humans was assumed to be 70 kg. The allometric exponents used for determining clearances and volumes of distributions were 0.75 and 1, respectively. The predicted human clearance (CL) was 2068 mL/hr; central volume was 2958 mL; peripheral volume was 9485 mL and 1195 mL for compartments 2 and 3, respectively; and intercompartmental clearance was 187 mL/hr and 183 mL/hr for compartments 1 and 2, and 1 and 3, respectively (Table 6).

The lowest EC₂₀ obtained from an in vitro tumor cell lysis assay was 0.097 ng/mL. The EC₅₀ concentration obtained in the same study was 2.03 ng/mL.

Accordingly, the projected human dose to maintain the maximal serum concentration of MEDI-565 below EC₂₀ value (0.097 ng/mL) is 0.75 μg of MEDI-565 administered as a 3-hour IV infusion once daily for 5 consecutive days.

The projected human dose to maintain the minimal serum concentration of MEDI-565 above EC₅₀ (2.03 ng/mL) is 1.5 mg of MEDI-565 administered as a 3-hour IV infusion once daily for 5 consecutive days.

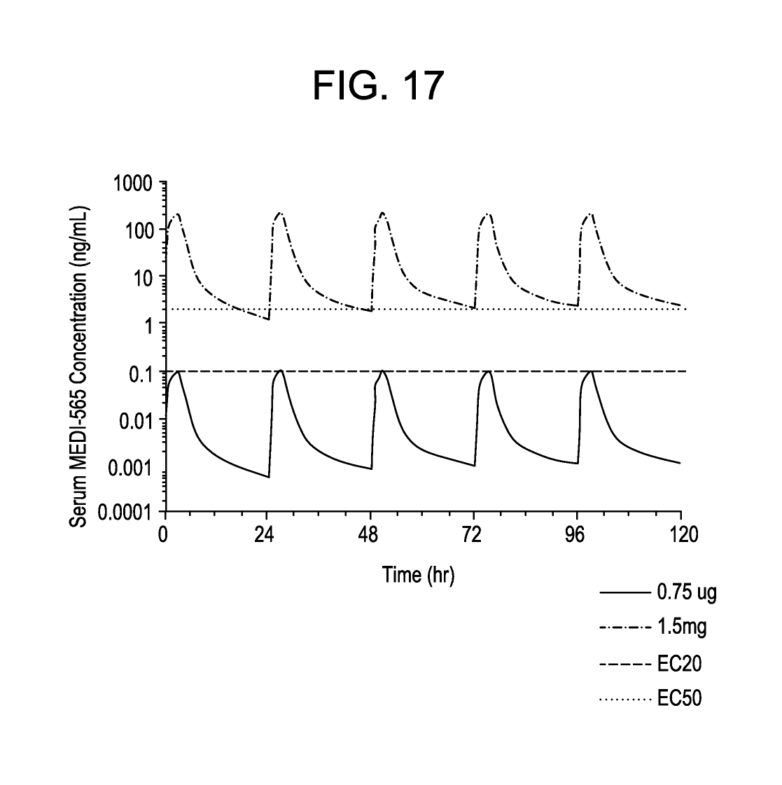

The simulated human serum concentration-time profiles for MEDI-565 following 0.75 μg or 1.5 mg of MEDI-565 administered as a 3-hour IV infusion once daily for 5 consecutive days are shown in FIG. 17.

### Materials and Methods:

The following materials and methods are exemplary of the methods used in the above examples.

### 1. Test Item—MEDI-565

MEDI-565 was constructed by standard DNA technologies and produced in Chinese hamster ovary (CHO) cells.

### 2. Control Item—Control BiTE® Antibody

Control BiTE® antibody (also known as MEC14 BiTE® antibody) contains the same CD3-binding arm as MEDI-565, but has a different target-binding arm that recognizes a small molecule herbicide, mecoprop, which is a structure completely absent in humans. Control BiTE® antibody was constructed by standard DNA technologies and produced in CHO cells.

### 3. Cell Culture

Dhfr-CHO (DSMZ, #ACC126) and MKN45 (DSMZ, #ACC409) cells were obtained from the German Collection of Microorganisms and Cell Cultures (DSMZ). ASPC-1 (ATCC, #CRL-1682), BxPC3 (ATCC, #CRL-1678), and A549 (ATCC, #CCL-185) cells were obtained from American Type Culture Collection (ATCC).

Dhfr-CHO cells were cultured in HyQ medium (HyClone, #SH30359.02) supplemented with 10 μg/mL adenosine (Sigma, #A9251), 10 μg/mL 2′ deoxyadenosine (Sigma, #D6000), and 10 μg/mL thymidine (Sigma, #T9250). CHO cells stably expressing human CEA (CHO/huCEA; 340000±180000 binding sites per cell) were generated by transfecting cells with plasmids containing the cDNA for human CEA. Transfected CHO cells were cultured in HyQ medium at 37° C. in a 5% CO₂ chamber.

All other cells were cultured in RPMI-1640 medium (Biochrom AG, #FG1215) supplemented with 10% heat-inactivated fetal bovine serum (FBS) (Gibco, #10270-106), and 100 U/mL penicillin/streptomycin (Sigma, 100 μg/mL, #P4333) at 37° C. in a 5% CO₂ chamber. The adherent cells were detached using 1× Trypsin-EDTA solution [Gibco, #35400; diluted in PBS (Invitrogen, #20012-043)].

ASPC-1 cells (ATCC, #CRL-1682) were obtained from American Type Culture Collection (ATCC). Cells were cultured in RPMI-1640 medium (Biochrom AG, #FG1215) supplemented with 10% heat-inactivated FBS (Gibco, #10270-106) and 100 U/mL penicillin/streptomycin (Biochrom AG, 10,000 μg/mL, #A2213) at 37° C. in a 5% CO₂ chamber. The adherent cells were detached using 1× Trypsin-EDTA solution (Gibco, #15400-054; diluted in phosphate-buffered saline [PBS; Gibco, #14190-094-043]).

### 4. Test System—ASPC-1 Cancer Cell Line

The human pancreatic cancer cell line ASPC-1 was used as the target cell population in cytotoxicity assays. This cell line was chosen for the MABEL studies as it was the most sensitive to MEDI-565-induced lysis, it naturally expresses human CEA at a density of about 90000 binding sites per cell, and it is well suited for FACS-based analysis.

### 5. Target Cell Labeling

For the analysis of cell lysis in flow cytometry assays, the fluorescent membrane dye 3, 3′-dioctadecyloxacarbocyanine or DiOC₁₈ (DiO; Molecular Probes, #V22886) was used to label target cells and to distinguish them from effector cells. Briefly, cells were harvested, washed once with PBS and adjusted to 1×10⁶ cells/mL in PBS containing 2% (v/v) FBS and the membrane dye DiO (5 μL/1×10⁶ cells). After incubation for 1 minute at 37° C., cells were washed twice with RPMI-1640 (Biochrom AG, #FG1215) supplemented with 10% heat-inactivated FBS (Gibco, #10270-106), 1× non essential amino acids (Biochrom AG, #40293), 10 mM Hepes buffer (Biochrome AG, #L1613), 50 μM β-mercaptoethanol (Gibco, #31350-010), 1 mM sodium pyruvate (Sigma, #S8636), and 100 U/mL penicillin/streptomycin (Biochrom AG, 10000 μg/mL, #A2213), otherwise known as RPMI complete medium. The cell number was adjusted to 1.25×10⁵ cells/mL. The viability of cells was determined using 0.5% (v/v) isotonic EosinG solution (Roth, #X833.1).

### 6. Isolation of Effector Cells

Human peripheral blood mononuclear cells (PBMCs) were isolated from the blood of healthy donors by Biocoll (Biochrom AG, #L6115) density gradient centrifugation using standard procedures. Erythrolysis was performed with lysis buffer (155 mM NH₄C1, 10 mM KHCO₃, 100 μM EDTA) for 4 minutes at room temperature. The plasma fraction was collected after the density gradient centrifugation, centrifuged again, and the supernatant used as donor-matched plasma.

### 7. Depletion of CD14 Positive Cells

When monocytes phagocytize dead tumor cells, they become positive for the membrane dye used for target cell labeling. Due to their similar forward scatter (FSC)-side scatter (SSC) appearance, they hardly differ from the living target cells. Thus, CD14+ cells were depleted from PBMC preparations (PBMC without CD14) using human CD14 MicroBeads (Milteny Biotec, MACS, #130-050-201) to facilitate fluorescence activated cell sorting (FACS)-based analysis of target cell lysis. Briefly, PBMCs were resuspended in MACS isolation buffer (every 10⁷ cells in 80 μL FACS buffer; PBS [Invitrogen, #20012-043], 0.5% (v/v) FBS [Gibco, #10270-106], 2 mM EDTA [Sigma-Aldrich, #E-6511]). CD14 MicroBeads (10 μL/10⁷ cells) were added and incubated for 15 minutes at 4 to 8° C. The cells were washed and then resuspended in Magnetic Cell Separation (MACS) buffer (500 μL/108 cells). The cell suspension was transferred to LS Columns (Miltenyi Biotec, #130-042-401), and CD14-negative cells were eluted with 3 mL MACS isolation buffer. PBMCs without CD14 were washed once in PBS (Invitrogen, #20012-043) and cultivated over night in RPMI complete medium at 37° C. in an incubator using T175 cell culture bottles. In indicated assays FBS was replaced by donors' own plasma for over night culture. Cells were thereafter washed once with PBS (Invitrogen, #20012-043) and then adjusted to 1.25×10⁶ cells/mL in RPMI complete medium.

### 8. Enrichment of CD3+ Cells

CD3-positive cells were enriched from human PBMC (PBMC with CD3ε) using the Pan T cell isolation kit (Miltenyi Biotech, #130-091-156) according to the manufacturer's instructions. Briefly, PBMCs were resuspended in Magnetic Cell Separation (MACS®) isolation buffer and stained with the provided Biotin-labeled antibody cocktail (10 μL/1×10⁷ cells) for 10 minutes at 4° C. Thereafter, for each set of 1×10⁷ cells, 30 μL of buffer and 10 μL anti-biotin microbeads were added. After an additional incubation of 15 minutes at 4° C., cells were washed and resuspended in 500 μL wash-buffer for up to 1×10⁸ cells. CD3-positive cells were then isolated using LS Columns (Miltenyi Biotec, #130-042-401). The cells were washed once with PBS (Gibco, #14190-094-043) and cultivated overnight in RPMI complete medium at 37° C. in an incubator using T25 cell culture bottles. FBS was replaced by donor-matched plasma for over night culture. Cells were then washed once with PBS (Gibco, #14190-094-043) and adjusted to a concentration of 1.25×10⁶ cells/mL in RPMI complete medium.

A second method was used to isolate and enrich human CD3+ cells from PBMCs of healthy donors. A volume of 1 mL RosetteSep T cell enrichment product was added per 20 mL of whole blood, followed by a 20-minute incubation. Subsequent isolation of CD3+ cells was achieved by density gradient centrifugation using RosetteSep DM-L density medium. After centrifugation, the cells were washed with PBS and resuspended in RPMI complete medium.

### 9. FACS-Based Cytotoxicity and T Cell Activation Assay

This assay was designed to quantify tumor cell lysis and T cell activation status of human effector cells in the presence of serial dilutions of MEDI-565.

Equal volumes of DiO-labeled target cells and effector cells from different donors (PBMC with CD3ε) were mixed, resulting in an E:T cell ratio of 5:1. A volume (160 μL) of this suspension was transferred to each well of a 96-well plate. Forty microliters of serial dilutions of MEDI-565, the control BiTE® antibody, or RPMI complete medium, as negative control, were added. Additional negative controls were target cells co-incubated with serial dilutions of MEDI-565, the control BiTE® antibody, or RPMI complete medium, and T cells incubated with RPMI complete medium. The BiTE® antibody-mediated cytotoxic reaction proceeded for 48 hours at 37° C. in a 5% CO₂ humidified incubator. Medium was removed before measurement of cytotoxicity, and was frozen at −80° C. for cytokine analysis. Staining of cell surface markers was carried out using directly-conjugated molecular antibodies (mAbs) for human antigens (anti CD4 [clone RPA-T4, #341115], CD8 [clone SK-1, #557834], CD69 [clone FN50, #555531], CD25 [clone 3G10, #MHCD2505] and CD3 [clone SP34-2, #345765]). Apart from anti-human CD25, which came from Invitrogen, Frankfurt, Germany all other antibodies were obtained from BD Bioscience, Heidelberg, Germany.

Cells were washed once in FACS buffer (PBS, 1% FBS, 0.02% NaN₃) and incubated at 4° C. in 50 μL for 30 minutes. Loss of target cell membrane integrity was monitored by adding PI at a final concentration of 1 μg/mL. PI is a membrane impermeable dye that is excluded from viable cells, whereas it is taken up by dead cells and becomes identifiable by fluorescent emission. Samples were measured by flow cytometry on a FACSCanto II instrument and analyzed by FACSDiva software (both from Becton Dickinson). T cells were identified by size, granularity, and expression of the surface marker CD4 or CD8. CD25-positive or CD69-positive T cells were classified as activated T cells, the percentage of which was calculated according to the following formula:

$$\frac{C D 23}{C D 69} positive T cells [ \% ] = \frac{{n}_{activated T cells}}{{n}_{T cells}} \times {100}_{r}$$

where n=number of events

Target cells were identified as DiO-positive cells. PI-negative target cells were classified as living target cells. Percentage of cytotoxicity was calculated according to the following formula:

$$Cytotoxicity [ \% ] = \frac{{n}_{deed target cells}}{{n}_{target cells}} \times 100 ,$$

where n=number of events.

### 10. Determination of Cytokine Content

The supernatant of each sample was stored at less than −65° C. until cytokine levels were measured using the commercial Human Cytokine/Chemokine Milliplex™ MAP Kit (Millipore Corporation, Billerica, Mass.) and Luminex® xMAP technology platform (Luminex Corp., Austin, Tex.). Reference standard curve, quality control (QC), and supernatant test samples were incubated with anti-cytokine antibody capture beads overnight at 2 to 8° C. in a 96-well filter plate. Plate wells were washed after the incubation to remove excess supernatant components. Cytokines captured on the beads were detected by incubation with biotin-conjugated anti-cytokine detection antibodies for 1 hour at room temperature followed by addition of streptavidin-phycoerythrin (SA-PE) reagent for 30 minutes at room temperature. Unbound detection antibodies and SA-PE reagent were removed by washing, the beads were resuspended in Luminex Sheath Fluid (Luminex Corp.), and the median fluorescence intensity (MFI) of each well was measured with a Luminex xMAP 200 System. Reference standard curves for individual cytokines were plotted by Softmax Pro GxP v5.2 software (Molecular Devices Sunnyvale, Calif.) and used to calculate cytokine concentrations (pg/mL) in QC and test samples within a respective plate. The MFI for each cytokine in a sample is proportional to its concentration within that sample. The lower limit of detection for all cytokines in the method was 3.2 pg/mL.

### 11. Data Analysis

Using GraphPad Prism 4 software (Graph Pad Software, San Diego), the percentage of cytotoxicity, CD25 or CD69-positive T cells, and the cytokine content were plotted against BiTE® antibody concentrations. Dose response curves of each donor were analyzed with a four parametric logistic regression model for evaluation of sigmoid dose response curves with variable Hill slope, and EC₅₀ and EC₂₀ values were calculated. Only curves fulfilling special data inclusion criteria were included in the calculation of mean EC₅₀ and EC₂₀ values, and thus, MABEL calculation. The inclusion criteria were: (1) a maximal response higher than 30% (only necessary for lysis); (2) an R₂ of the curve fit over 0.95; (3) a valid upper and lower plateau of the fitted curves (Hill slope of the linear regression through the first and last three data points should not differ significantly from zero); (4) an EC₅₀ and Hill slope of the fitted curve within 90% (nonsimultaneous) tolerance interval constructed at a 95% level of confidence; (5) normal distributed residuals (p>0.05); and (6) all Studentized residuals of the fitted curves were between −3 and 3. To match the 6^{th} condition, individual points could be disqualified (2 middle responses or 3 individual points in either plateau). All inclusion criteria were tested using different analysis methods embedded in GraphPad Prism 4 software (Graph Pad Software, San Diego).

### 12. Calculation of Receptor Occupancy

The amount of a monoclonal antibody (mAb) bound to its receptor can be estimated from the following binding relationship:

Receptor(A)+mAb(B) [symbol] receptor−mAb complex(C) Equation 1:

The binding dissociation constant (K_{D}) of the respective antibody is represented by:

$${K}_{D} = \frac{[ receptor ] \times [ mAb ]}{[ receptor - mAb ]} \qquad Equation 2$$

Finally, the fractional occupancy, fraction (F) of all receptor molecules that are bound to the antibody, can be calculated by:

$$F = \frac{[ receptor - mAb ]}{[ receptor ] + [ receptor - mAb ]} \qquad Equation 3$$

Formation of equation 2 and substitution into equation 3 results in:

$$F = \frac{\frac{[ receptor ] \times [ mAb ]}{{K}_{D}}}{[ receptor ] + ( [ receptor ] \times \frac{[ mAb ]}{{K}_{D}} )} \qquad Equation 4$$

Simplification of equation 4 results in:

$$F = \frac{[ mAb ]}{[ mAb ] + {K}_{D}} \qquad Equation 5$$

Therefore, at equilibrium condition, the fraction of all receptor molecules that are bound to the antibody can be calculated if the concentration of mAb and the dissociation constant K_{D} of the respective antibody are known.

TABLE 1
EC₂₀ Values of Specific Lysis and CD69 and CD25 De Novo Expression for MEDI-565

| | Lysis | CD69 | CD25 | Lysis | CD69 | CD25 | Lysis | CD69 | CD25 | Lysis | CD69 | CD25 |
|---|---|---|---|---|---|---|---|---|---|---|---|---|
| | A/P | A/P | A/P | C/3 | C/3 | C/3 | A/3 | A/3 | A/3 | C/P | C/P | C/P |
| Number of values | 20 | 22 | 24 | 10 | 1 | [symbol] | [symbol] | 11 | 10 | 7 | [symbol] | [symbol] |
| Mean | 0. [symbol] | 0.207 [symbol] | 0. [symbol] | 0.4 [symbol] | 0.0 [symbol] | 0.2 [symbol] 7 | 0.0 [symbol] | 0.1053 | 0.1 [symbol] 3 | 0.3706 | 0.1342 | 0.2169 |
| Std. Deviation | 0. [symbol] | 0. [symbol] 21 | 0.2 [symbol] | 0.27 [symbol] | 0.0 | 0.12 [symbol] 0 | 0.0 [symbol] | 0.0 [symbol] | 0.0 [symbol] | 0.2239 | 0.13 [symbol] | 0.13 [symbol] |
| Std. Error | 0. [symbol] | 0.0 [symbol] 42 | 0. [symbol] | 0.0 [symbol] | 0.0 | 0.04 [symbol] | 0.01 [symbol] | 0.0 [symbol] 2 | 0.02 [symbol] | 0.0 [symbol] 464 | 0.04 [symbol] | 0.04659 |

[symbol]

indicates data missing or illegible when filed

TABLE 2
EC₅₀ Values of Specific Lysis and CD69 and CD25 De Novo Expression for MEDI-565

| | Lysis | CD69 | CD25 | Lysis | CD69 | CD25 | Lysis | CD69 | CD25 | Lysis | CD69 | CD25 |
|---|---|---|---|---|---|---|---|---|---|---|---|---|
| | A/P | A/P | A/P | C/3 | C/3 | C/3 | A/3 | A/3 | A/3 | C/P | C/P | C/P |
| Number of values | 20 | 20 | 22 | 10 | 1 | [symbol] | 11 | 11 | 11 | 7 | 2 | [symbol] |
| Mean | 0.9710 | 4.0 [symbol] | 2. [symbol] | 5. [symbol] | 1.340 | [symbol] | 0.37 [symbol] | 1.260 | 1 [symbol] | 3. [symbol] | 2.52 [symbol] | [symbol] |
| Std. Deviation | 1. [symbol] | 3. [symbol] | 1.7 [symbol] | 3. [symbol] 4 | 0.0 | 4.0 | 0.1 [symbol] | 0. [symbol] | 0. [symbol] | 3. [symbol] | 0. [symbol] | [symbol] |
| Std. Error | 0.40 [symbol] | 0. [symbol] | 0. [symbol] | 1.222 | 0.0 | 1.442 | 0.0 [symbol] | 0. [symbol] | 0. [symbol] | 1.2 [symbol] | 0.4334 | [symbol] |

Lysis A/P = Lysis of ASPC-1 cocultured with PBMC w/o CD14+
CD69 A/P = CD69 expression on PMBC w/o CD14 cocultured with ASPC-1
CD25 A/P = CD25 expression on PMBC w/o CD14 cocultured with ASPC-1
Lysis A/3 = Lysis of ASPC-1 cocultured with PBMC w/o CD3E
CD69 A/3 = CD69 expression on PMBC w/CD3 cocultured with ASPC-1
CD25 A/P = CD25 expression on PMBC w/CD3 cocultured with ASPC-1
Lysis C/3 = Lysis of CHO/CEA cocultured with PBMC w/CD3E
CD69 C/3 = CD69 expression on PMBC w/CD3E cocultured with CHO/CEA
CD25 C/3 = CD25 expression on PMBC w/CD3 cocultured with CHO/CEA
Lysis C/P = Lysis of CHO/CEA cocultured with PBMC w/o CD14
CD69 C/P = CD69 expression on PMBC w/o CD14 cocultured with CHO/CEA
CD25 C/P = CD25 expression on PMBC w/o CD14 cocultured with CHO/CEA
All values stated are in [symbol] /mL [symbol]
indicates data missing or illegible when filed

TABLE 3
EC₅₀ and EC₂₀ Values of T Cell Activation and Specific Tumor
Cell Lysis Mediated by NIEDI-565

| | Specific | EC₅₀ | | Specific | EC₂₀ | |
|---|---|---|---|---|---|---|
| [pg/mL] | Lysis | CD69 | CD25 | Lysis | CD69 | CD25 |
| Number of values | 10 | 5 | 10 | 10 | 5 | 10 |
| Mean | 2032 | 13770 | 4652 | 97 | 441 | 376 |
| Std. Deviation | 2286 | 8864 | 2853 | 83 | 325 | 183 |
| Std. Error | 723 | 3964 | 902 | 26 | 146 | 58 |
| Lower 95% Cl* of Mean | 397 | 2764 | 2610 | 38 | 37 | 248 |
| Upper 95% Cl of Mean | 3668 | 24780 | 6693 | 156 | 845 | 509 |

TABLE 4
EC₅₀ and EC₂₀ Values of Cytokine Release Mediated by MEDI-565

| | EC₅₀ | | | | | EC₂₀ | | | | |
|---|---|---|---|---|---|---|---|---|---|---|
| [pg/mL] | IL2 | IL6 | IL10 | TNFα | IFNγ | IL2 | IL6 | IL10 | TNFα | IFNγ |
| Number of values | 9 | 5 | 9 | 7 | 9 | 9 | 7 | 9 | 10 | 9 |
| Mean | 2143 | 4038 | 3918 | 6564 | 3287 | 731.5 | 1835 | 686.4 | 1286 | 1556 |
| Std. Deviation | 3035 | 5560 | 4499 | 5950 | 3992 | 211 .6 | 258.6 | 976 | 996.2 | 2852 |
| Std. Error | 1042 | 0.486 | 1500 | 2249 | 1331 | 70.55 | 977.3 | 325.3 | 315 | 950.8 |
| Lower 95% Cl* of Mean | −0.19 | −2.866 | 0.458 | 1.061 | 0.2185 | 0.569 | −0.556 | −0.639 | 0.573 | −0.6369 |
| Upper 95% Cl of Mean | 4.476 | 10.94 | 7.375 | 12.07 | 6.355 | 0.894 | 4.227 | 1.437 | 1.998 | 3.748 |

*Cl: Confidence interval

TABLE 5
Pharmacokinetics and Bioavailability
Study of MEDI-565 in Cynomolgus

| | No. | Dose | Dose | Dose | Route of | |
|---|---|---|---|---|---|---|
| | of | Level | Volume | Concentration | Administration | |
| Group | Males | (mg/kg) | (mL/kg) | (mg/mL) | Day 1 | Day 8 |
| 1 | 3 | 0.5 | 1 | 0.5 | IV | SC |
| 2 | 3 | 0.5 | 1 | 0.5 | SC | IV |

Monkeys
IV = intravenous;
No. = number;
SC = subcutaneous

TABLE 6
Summary of Pharmacokinetics Parameters of MEDI-565 Determined in
Cynomolgus Monkeys and Predicted for Humans

| PK Parameter | Units | Cynomolgus monkey | Human |
|---|---|---|---|
| Clearance (CL) | mL/hr | 221 | 2068 |
| Intercompartmental CL for | mL/hr | 20 | 187 |
| compartments 1 and 2 | | | |
| Intercompartmental CL for | mL/hr | 19.6 | 183 |
| compartments 1 and 3 | | | |
| Central volume | mL | 150 | 2958 |
| Peripheral volume for | mL | 481 | 9485 |
| compartment 2 | | | |
| Peripheral volume for | mL | 60.6 | 1195 |
| compartment 3 | | | |

### INCORPORATION BY REFERENCE

All publications and patents mentioned herein are hereby incorporated by reference in their entirety as if each individual publication or patent was specifically and individually indicated to be incorporated by reference. In case of conflict, the present application, including any definitions herein, will control.

### EQUIVALENTS

While specific embodiments of the subject disclosure have been discussed, the above specification is illustrative and not restrictive. Many variations of the disclosure will become apparent to those skilled in the art upon review of this specification and the claims below. The full scope of the disclosure should be determined by reference to the claims, along with their full scope of equivalents, and the specification, along with such variations.

### REFERENCES

Barnett et al., Tumor Localization Using Monoclonal Antibodies. II. Biodistribution of A5B7/111 indium chelates. Int J Cancer Suppl. 1988; 3: 34-37.
Boxer et al., Localisation of monoclonal antibodies reacting with different epitopes on carcinoembryonic antigen (CEA)—implications for targeted therapy. British J. Cancer. 1994; 69: 307-314.
Duffy M J. Carcinoembryonic antigen as a marker for colorectal cancer: is it clinically useful?Clin Chem. 2001; 47: 624-30.
Eisenhauer E A, Therasse P, Bogaerts J, Schwartz L H, Sargent D, Ford R, et al. New response evaluation criteria in solid tumours: revised RECIST guideline (version 1.1). Eur J. Cancer. 2009; January; 45(2): 228-47.
Fogar P, Sperti C, Basso D, Sanzari M C, Greco E, Davoli C, et al. Decreased total lymphocyte counts in pancreatic cancer: an index of adverse outcome. Pancreas. 2006; January; 32(1): 22-8.
Galon J, Costes A, Sanchez-Cabo F, Kirilovsky A, Mlecnik B, Lagorce-Pagès C, et al. Type, density, and location of immune cells within human colorectal tumors predict clinical outcome. Science. 2006; Sep. 29; 313(5795): 1960-4.
Fidarova et al., Microdistribution of Targeted, Fluorescently Labeled Anti-Carcinoembryonic Antigen Antibody in Metastatic Colorectal Cancer: Implications for Radioimmunotherapy, Clin Cancer Res. 2008; 14: 2639-2646.
Hammarstrom S. The carcinoembryonic antigen (CEA) family: structures, suggested functions and expression in normal and malignant tissues. Semin Cancer Biol. 1999; 9: 67-81.
Harwood et al., Mapping epitope characteristics on carcinoembryonic antigen. British J. Cancer. 1986, 54: 75-82.
Kabat et al., Sequences of Proteins of Immunological Interest, 5th Ed. Public Health Service, National Institutes of Health, Bethesda, Md. 1991.
Locker G Y, Hamilton S, et al. ASCO 2006 update of recommendations for the use of tumor markers in gastrointestinal cancer. J Clin Oncol. 2006; 24: 5313-27.
Lutterbuese R, Raum T, Kischel R, Lutterbuese P, Schlereth B, Schaller E, et al. Potent control of tumor growth by CEA/CD3-bispecific single-chain antibody constructs that are not competitively inhibited by soluble CEA. J. Immunother. 2009; May; 32(4); 341-52.
Nap et al., Specificity and Affinity of Monoclonal Antibodies Against Carcinoembryonic Antigen. Cancer Res. 1992; 52: 2329-2339.
Osada et al. Metastatic colorectal cancer cells from patients previously treated with chemotherapy are sensitive to T-cell killing mediated by CEA/CD3-bispecific T-cell-engaging BiTE antibody. British Journal of Cancer, 2010, 102: 124-33,
Rother M. Carcinoembryonic antigen monitoring for early detection of asymptomatic incurable metastatic colorectal cancer. J Clin Oncol. 2007; 25: 1293-4; author reply 1294.
Sanders D S, Evans A T, Allen C A, Bryant F J, Johnson G D, Hopkins J, et al. Classification of CEA-related positivity in primary and metastatic malignant melanoma. J. Pathol. 1994; April; 172(4): 343-8.
Suzuki T, Richards S M, Liu S, Jensen R V, Sullivan D A. Influence of sex on gene expression in human corneal epithelial cells. Mol. Vis. 2009; 15:2554-69.
Tendler A, Kaufman H L, Kadish A S. Increased carcinoembryonic antigen expression in cervical intraepithelial neoplasia grade 3 and in cervical squamous cell carcinoma. Hum Pathol. 2000 November; 31(11):1357-62.
Simon R, Freidlin B, Rubinstein L, Arbuck S G, Collins J, Christian M C, et al. Accelerated titration designs for phase I clinical trials in oncology. J Natl Cancer Inst. 1997; August 6; 89(15): 1138-47.
von Bernstorff W, Voss M, Freichel S, Schmid A, Vogel I, Jöhnk C, et al. Systemic and local immunosuppression in pancreatic cancer patients. Clin Cancer Res. 2001; March; 7(3 Suppl): 925s-932s.

1. A method for treating a CEA-expressing cancer, comprising administering to a human patient in need of treatment a protein composition, which protein composition comprises an antibody comprising a first binding domain that binds to human CD3 and a second binding domain that binds to human CEA, which antibody is provided at a dose of 1.5 mg per day, 1.5 mg to 3 mg per day, 3 mg to 5 mg per day, 5 mg to 7.5 mg per day or 7.5 mg to 10 mg per day on a dosing schedule comprising administering the protein composition once per day for at least one day.

2. The method of claim 1, wherein the dosing schedule is part of a treatment cycle of 21 or 28 days.

3. The method of claim 2, wherein the CEA-expressing cancer is chosen from: colon cancer, ovarian cancer, prostate cancer, rectal cancer, pancreatic cancer, esophageal cancer, stomach cancer, lung cancer and breast cancer.

4. The method of claim 3, wherein the CEA-expressing cancer is a relapsed or refractory cancer.

5. The method of claim 3, wherein the CEA-expressing cancer is an adenocarcinoma of gastrointestinal origin.

6. The method of claim 3, wherein the antibody is a bispecific single chain antibody comprising a first binding domain that binds to human CD3 and a second binding domain that binds to the human CEA.

7. The method of claim 6, wherein the bispecific single chain antibody comprises an amino acid sequence chosen from the amino acid sequences of SEQ ID NOs: 28-44 and 46-52.

8. The method of claim 6, wherein the bispecific single chain antibody comprises the amino acid sequence of SEQ ID NO: 48.

9. The method of claim 6, wherein the bispecific single chain antibody comprises the amino acid sequence of SEQ ID NO: 49.

10. The method of claim 6, wherein the bispecific single chain antibody comprises the amino acid sequence of SEQ ID NO: 46.

11. The method of claim 6, wherein the bispecific single chain antibody comprises the amino acid sequence of SEQ ID NO: 51.

12-23. (canceled)

24. The method of claim 7, wherein the protein composition is administered intravenously.

25. (canceled)

26. The method of claim 7, wherein the protein composition is administered on a dosing schedule comprising administering the protein composition once per day for at least 3 or 5 consecutive days.

27. (canceled)

28. The method of claim 26, further comprising one or more additional treatment cycles of 28 days, wherein the protein composition is administered on a dosing schedule comprising administering the protein composition once per day for at least one day per each treatment cycle.

29. The method of claim 28, wherein the protein composition is administered on a dosing schedule comprising administering the protein composition once per day for at least 3 or 5 consecutive days per each treatment cycle.

30-35. (canceled)

36. The method of claim 1, wherein the patient receives a therapeutically effective dose sufficient to: lyse at least about 60% of the cancerous cells that express CEA; increase release of one or more pro-inflammatory cytokines, perforin, and/or granzyme by at least about 50% relative to untreated cells; reduce tumor volume by at least about 25%, as compared to untreated control tumors, increase expression of T cell activation markers CD69 and CD25 by at least about 25%>, relative to untreated cells; and/or induce proliferation of CD3+ T cells of peripheral blood mononuclear cells.

37-47. (canceled)

48. The method of any of claim 1 or 2, wherein the dosing schedule maintains the antibody at a serum concentration between about 0.1 ng/mL to about 2 ng/mL in the patient for at least 4 hours or 1 week.

49-66. (canceled)

67. A method of treating a CEA-expressing cancer, comprising administering to a patient in need thereof a composition comprising an antibody comprising a first binding domain that binds to human CD3 and a second binding domain that binds to human CEA at a dose of antibody and on a dosing schedule sufficient to maintain a serum concentration of antibody that that is therapeutically effective and sufficient to lyse at least about 60% of the cancerous cells that express CEA.

68-83. (canceled)
